SEQUENCE LISTING (483 sequences)

SEQ ID NO: 1 - DNA, 21000 bp, Homo sapiens
gtaaattgctgggccctcagccatggttactgtcaggccagcccaggaaggacactggtttcaggtgacctgggtgaggtggaggcggtggggggggttcctgggaccaggccgccattgcaaaaacaaacggcctgcttgtacctgctatcgccctctaagcttcaggtggtgggtggggctgaggggctgagaggtggccccaaagaaagacaaccacatcctaaaccctggaacccgtgaacaaggcttcatttggaacaccagtctttgaaggtaccattaagtgtcttgagatgagatcatcctggagtctgcaggtagaccctaaattccgcgacatacattttattttttatttttacttttttttttgagatagcgtgtcactctgttgcccaggctggagtacagtggcgtaatctaggctcactgcaacctccgcctcccgcgttcaagtgattctcccgcctcagtctcccgagtagctgggattacaggagcaaaccaccacgcccggcaaatttttgtatttttagtggaaacgggctttcaccatgttggccaggatagtctcaggctcttcaccttgtgatccgcctgcctcggccttccaaagtgctgggatgacaggtgtttattttcgtttttgttttttagagacagagtttcactcctgttgcccaggctggagtgcaatggcgcaatctcggctcactgcaacctccgcctcccgggttcaagtgattctcttgcctcagcctcccaaggagctgggattatagggcatgcaccaccacgcctggctaatttttgtatttttggtggagaggaggtttctccatgttggccaggctggtctcgaactctgacctcaggtgatccactcacctcggcctgccagagtgctgggattacaggtgtgagccattgcacccggccagacactgtcttaaaaaaataaaaataaaaaaaggtactggacaggtggctggtcatgtggggcttgcggggtaagggatggccttgggtttatactttgagcaagacaggaggcctggcggggccacgatcagaagtgggttttaacacgattccgaagggttagactggtgcttggaacctggtggtctctactgctgccagctgccactcagacccccggccgcccctgcctgtctgctttcagggcggggcccatcccaggcactccgcgaacagacagaagcggctggttctggtggtgtgaggatgggggtctccaggggagcaaatccacagtaacacactgggcttctcccagatccaccggacattcctgatgagtgaccctggctgcagatggctcttggtgagtctccgttgaccgggtggttcgggccaaaggaccttgccgtgcgatgcctccatggagctcggcggccacccctaatccgggctggaatcctctggcaccagacgcccggcggggctccaaggctcctgggcccacgccaggccccggcctgaaacgcagcactgctggaataaaacacgccagagaagcagggcgctaggctcgcctggatttggggaaacgagaattgtcatgtcttctccctccgtaggttggtgttaatgaaaaacccccttttcagcatggactaaatcccgctaattctatttagggagcagtgtctactccgggccgggaactgctccggtttccaaccgcagcctcccgtgagggtcccagtgtggagggccggggtcatcccctcactctgggatggctcccaacccgctgacctggcctccccgtcaggcaccctcacagctctgtcagactcttcatgtgatcagcgctcctgggaacattagtttagggtgttttctgctgctgggatggtggcccgggagggaccctcctggtgaccctgctgggcctggccctcagtttccccagccgtaggctggcagcctcgaactgtacgagctccaggccacagtttccccagctgtaggtcagtgggccagggactgtagggggctcgggcctcagtttccccagctgtaggcgggtggcttgggcctcagtttccccacctgtaggcgggcggccttggattgtatggagcccgggcctcagtttctccagctgtaggcggatggccgtggactgtgtgtggcctgggcctcagtttcccccgctgtaggcagatggccgtggactgtgtgtggctttggcctcagtttccccagctgtaggcggatggccgtggactgtgtgtggcccggacctcagcttccccagctttaggcggttggccgcggactgtgcaaagtccaggcctcagtttccccagctgtaagcgggcggccgcccactgtacaaggtccaggcctcagtttccccagctgtaggtgagaggccgcggactgtacaaggtccaggcctcagtttccccagctgtaggcgggcggccaaggactgcaaggtccaggcctcagtttccccacctgtaggcgggcggccgcggactgtacaaggtccaggcctcagtttccccagctgtaggtgagaggccgcggactgtacaaggtccaggcctcagtttccccagctgtaggcgggcggccgcctactgtataaggtccaggcctcagtttccccagctgtaggcgggcggccgcggactgtacaaggtccaggcctcagttttcccagctgtaggcagccggccgcggactgtgcggggcccggacctcagtttccctatctccgcagtcgggattcgctgttccggggcgaacaaagaacggtcgggggtgaccccggatcatggcgcgcccgcagtatctgtggccactgtggaaacagccctggcgtgtccgggacgcgacgcggcctttccctccccgcgcgatcacccagatgtgagcgctcagtttgaatcggactttttggccatgtgcccacaaccaacatggagccctgagggtttcctgcccgactccaagatggcgatgcgactccctttgccctcacggcccaggcgggcgggtcctgcccacttgcaagatggcggcagcctgggcaaagcacgcttcccgccgcctgactcgccacgtacccactctcaagatggcgtctcccgcaaagccacgcgccagctgactggctcagacgccgtcacgtgatcccattttctccccctcccccgactacacaagccgtttccgccttcggccttgaggaataaccgcctcccccaataggcgcctgcgatttctctgggtcccgcccccgggcggccttctggccagtgggaggtgctggcgggggcccgcggggctctccccgccccctggctgcagcgggagccaatgggcagcgcgcgcgggggacgtgtgcgggcgtctccgccattttgtgagtctataactcggagccgttgggtcggttcctgctattccggcgcctccactccgtcccccgcgggtctgctctgtgtgccatggacgggtgagtcgcacgtcgccccgcgccccaccgccctccccgcgccgcagccctgcccccgccgccgcgccggcctccccggggccgatctcgccccctctcgggcgggagggggcggcgggctctccccttcctctccgcgtggcgggcgcgggtgcggggcgggtcaggggcttcccgggggtctggccgcgtccccatcccccgtccggcccgcttccggcccccgcgcgccccgtccctagcgcacgcggcctcccgcgcccctccccctccgcgcgcgtccccgcacttcgcctttgccccgcctcggcggcgcgcggcccttcccgcttcccgtccggccctcgcgcgcccggatggcccttcccgcctcccgtccgctcccctcgtgcgcctgcgtagcccgtcgcgcgtccccgttggccccagcgcgcgtgcgcggccgccatcgggggcgcgcgcctttcccgccgcccggactctgcccccggcgcagccccggaagcgcaggcggggctggggcggccgccgggagggaaagctggcgccggggcctcgccgcgggcccgagtctctgcgctccccgccctacccctgccccccgtgcgccccgggccgcggccaaggggtttcggtgatctcgggagagaagcggccgccccgagggtctgtcctccgccccggggccggaggggcctctccagggacccccgcgggcgccgtcatggggaggggccttcccggctctcgggccggggcagcctttccgggcggctttgctgggcgggcttcccttgggagaggtgggagccccctccagaaaggccgtgtcccgccgagcgcttcctgcgcgaggccctccctgggaataggaagcgggagaggctggagatgtttcgcattctttccgttttcctgagaagtttttcttgggttcagtcacttttaaaagtctgttaaacgtggtccgtgcagtctgtggtgtcagaagaggcgcagccctagagtgggtctggaccccttagcatgcgtgcgggcaggtgggtcctgcagacggcgagacgagggctccccgctctcggacccagcaccgcgggctcgggggccgcgcactcgcctcccggagtgaggcaggaggcccggaggctgcccttccgacggacgctccgagtcacttagggcctcgccgggagttgcagtggccgggagaagccgtggggagctcactgttgggtcaggacctggccggggctgaagggagggcctcggacagtccggctttctatccaccttccccccggggtgtcccgaggagggagagtcgggtcgggcggggctcgggttgatcagtaactgaggctgtgttctggtcggtctgttaatttttattgacggtttccacggccgcctcctgctctcctcggtggatcgggctccactctgctgagaggcaccctcttgggggcccacccctacccgggtggaagctctggcccaggcctgtgagtttgccagtttcagcctctccgaggcgtttccaacttactggaaagcacacagcagcccttgaaccctctcagcggcatctagcattcctcggggcttccgatgggggtgtctttgtcttggaacgtgtggggcctggtccagaggtggggcctggggtcctgctgggagggaggggcgccaaaggacaatggtggccccttttcaagttgcagtcaagtggacggctcacttccctgcccttctgagctttctctagcggggcctcgtgcagccagagggagccctgcggaggtggcagctgcagggacctacgggctctcctggcccggggacagctgggtgctcctgctgctgagtggccaccaggctaacgttgtctcctttctttctctctacagcattgtcccagatatagccgttggtacaaaggtaggcacttctcactttgcttctccgtgacgctcctctgaccttgtgccgaccccgggggccaccccccagcgctgttgcgttggctagagggcgcttttccattcctaaggggcactacccttggtctggaatctggagttgggcggtagggtttgaaccccgggctccagtgcccgtgagttttgggggcttgggtttgctggccgcccctcaggtctggaatctgagctgctcttcggttcttgctgtcagcagcgagggctctgtgtgccccaggcctgcctttggaacttctgcctctagaggctggaatcgtgccgttcagtgggaaacctcatgttgtggctgcctgggagactcttggttttgataggaaaagagccacctggctgtggaggggacctggatagaatccagcctatttgagtggctaggatgggcatttactggttgattttgacgttaagtttttttgtttttgtttttgttttttatttttgagatggagtcttgctttgtcaccaggctggagtgcagtggtgtgatcttggctcactgcaacctccacctctcaggttcaagcgattctcctgcctcaacctcctgagtagccgggattacaggtgcccaccaccatgcccagctaatttttgtattttttttttttttttttggtaaagatggggtttcgaacattgttggccaggctggtctggaacacaggtgatgcaccccccttggcctcccacagtgctgggattacagttgtgagccgccagacccggctgacattaagtatctttgatggcatctggttagaacagcagcaaactgtcaaaggtggtgtttggggcaagagaaaaatgaatgtgccacgtctctggggtgtgcgaatcttgcagccccatggagggtgaggccagggctcagcatgtgaggaaagggggtcagaacctggagaagaagaaggtcctggggaggcgattctcgagggagctgtagtcctcgcttagctggggtagttggttaatgattctgctggtcattgtgggttgagggggctcccgacttcagggcttgtgccccgagagcctcccgcagggcgagcccacagtggtggcgtcctgagccagcagccagttggtttctggtcagtcagcagtggtgcctgggccaggtttcggtttttcccaagtggactgctgtctttacgcgctgcctgccccgatcttcctggagataaaacctgtttgttcagtgtgggaggagggagaggtactttcatctgaagcatttgcaacaatccctagatgtgtctggcttgcaggcagtcgtctcttccctgcttctgtgggcctgctttccagctggagtggccctcagcttccatcgggtggtccttgagccctcctctgcagttcctagggggctcctgggctgggacgttgaagcccagccaggacttgaagcctgcgggagggattttgccacagggcagggttagggctcctgctgggtgcaggccctggtaggaggacggggtagtaccctgacccagatgataccccccctcccccccaccaccagggagccctcagctgacctgggcccacacctggctacgctgtcctttagcccaggacagagcaagctcttgttcagtgtggggttaaagacacctaggaactgtgctgtagggccccagctgcagactggaagtcctggggcgtttcctgtcaatctgtgtgcttcaggacaggtgccctgaagggaccctcctggggtacctggcagctgctgtgaaggcagtggaggcgctaggcccagctcgtgtctcctggttgctgccctgtggagcctcggagcctccaggtgtcccttccgttctgctggggcgccgttgagctgagccaagccggcctgtgccgcctgggcaggcatggagtttgctgccgcagacagttgtgggcaatgccaggccacttgcccatgggaatgagattagggcttcaggcaggcggccaagaggaccgctcagaggaagcggccccgcccaccactccagtcaaagacagggtccaagatgccctttttaaggtttgtgcagccagcacatgggagccgcatgggcgcttggccactctccagcttgggggaggaccagccctggtgtcctggacggggtcggctcctgggctcccccagcctctgagctggaggggagagcagacaggaagcgacttcctgtgggaggcgaaggttggaggccctgcgtctcaggtgcacccatcctcccttcctcccgtcctcccttcctccggtcctggcactgctgtgttttctgatcgcgtggtttccacctgaggcggctggctctgacccctttcccctgggcctgcgcagccccgccttgtgctcatagggggccccagactctgaaagcgagaggactcgagggtctgttttcagtatttggcttcatctgtgtccggccggaacctcagggccgccgtgacactggacactgttcattctctgaccctaaagtcttggggactgaacaggtgccatttgggtgggcgccgggcctgtggaccctgagtgattcctggttccggcccttgtggctgctcgattgcttgtttggtgacgccctgagtgcttgtgggattggtgggagactcagggctgcagttagaggtctcattggcaagggccagtccctgttccccatcctccttgagcaggtgccggagtctcccgaggctcttgccgtccgcgtcgggaggaggcagctgtggggctttggaggctcgggcagtcttccttccaggaagctcttgagccagggccagggcaggagtggagctgaacgtgggtggaggggaggcggccttaagagaagaccaagcgcaggctcggggaggccagcgttggtgggggctctttggccaggtgtgggggcagctccttgaccaggcatgggggtgtgtggagctgggggttgtctgaggggggacagggtgtgatatccactggttctttctgatgcgatggccctgggcccttcagcaggtgggggcaggctcttggcctcaatcctgatacgggagatggaggctcctttgttccctgggatggccctgtcctggggggcctgggcgttgctgccctcagcgcctgcagaggctcgctttgcagaccaagcgcctgtgctccaaggtcctcagacccgagctttgttttctgtgtaactaagggccgtaaccaacgtctcctccaatttcactctttgaataaacgtgtgtcttcaatccttgcagcgggggggatgtctgccgtcctgagcctaggatcagtctagtgcaattcttagaagttgatccaattacatagaaataaccagtgaatagattgtttctggagagaaaatggacaggtcagaagtgatggagccgctgtggagctcccccggccctagtcacggccgcgataccagcggatcactgggcgggaggccgtgtctctagtcctcagtctcgcgtgtgcagagacggaggtcgggtttcttttccgggctggaaaacaggagaggaggggctcctgcgagagtgagggaggcagcgctataatttggttctcttgcgaggccaccagaagtgaggacggctgcctgggccctgccgtgtgctgctgggggggctggctccatggagggggtcctctgccctgctggctggtggctgaggccccaggcctgtgcccagggccgctggcagcacagtgaggccacgtccacagcagagttggccgtggagatagtgtacgtggtgccctccgtagctgagagcctcccagccccacactggctgagtacccagggcttccagaatttggaaaaaggagcagtgttttggtcgtggagctgtggggtgccgtgcgcgtccatgggctgggtctgcgctcaggctgccctgccgtggaagtgtgggtgtgggtatgggttgggggtctgggaccccaggcagcccaagtgggagcagggccggccggtggggaagggaggctctggcctggtgggaagtgcagctccgcgttgtcccttctcttgcagcggggatctgacgagcttttctctacttgtgtcactaacggaccgtttatcatgagcagcaactcggcttctgcaggtaaggccgggactcggcccagggcaagacccgtgggtgtctttccctggaacaacgttacgttcttgttctgggactctactttccatctccaactgcaggggcccatggtccacgctacagacccaggtccaagttctcgctgcagagaatttccagggaggcgctggatggatggtccaggctggcacttggacctgtgggcgggttgtcaggagctgtcgggagcaggggtcctgactgtgcaggtcttggcctctcgcgctgctggttcacatgcaccctcctggggctcagggttggtctccgtagcaggcagggctctagggggatagcagggaccttcctctgtgggctcctgcgagttggtgcctgcatctcatggcacccccttttcagcaaacggaaatgacagcaagaagttcaaaggtgacagccgaagtgcaggcgtcccctctagagtgatccacatccggaagctccccatcgacgtcacggagggggaagtcatctccctggggctgccctttgggaaggtcaccaacctcctgatgctgaaggggaaaaaccaggtacctgagccgcgtttctccggggtgctcacaccgtgcaggcggggacgaggagggcccagcgctcactgcctccccaacaggccttcatcgagatgaacacggaggaggctgccaacaccatggtgaactactacacctcggtgacccctgtgctgcgcggccagcccatctacatccagttctccaaccacaaggagctgaagaccgacagctctcccaaccaggcggtgcgtggccccgcggcggaccccagcagcccggggacctcgggggtgggcccagccgcaggggccggggactcacggctgtgcctcccacagcgggcccaggcggccctgcaggcggtgaactcggtccagtcggggaacctggccttggctgcctcggcggcggccgtggacgcagggatggcgatggccgggcagagccccgtgctcaggatcatcgtggagaacctcttctaccctgtgaccctggatgtgctgcaccaggtgaggtggtcccatcaccgccagggcaggtcggctgctattgctgtgggcacaggcacacgggaggggcctggcagggctggtgggcaccgggcaggagctcatgctgtggcccgggacctgcagattttctccaagttcggcacagtgttgaagatcatcaccttcaccaagaacaaccagttccaggccctgctgcagtatgcggaccccgtgagcgcccagcacgccaagctggtgagtggggctcccgggacggcgcccgccctggccctggcccggcgacgtctcacggtccctctcccctcagtcgctggacgggcagaacatctacaacgcctgctgcacgctgcgcatcgacttttccaagctcaccagcctcaacgtcaagtacaacaatgacaagagccgtgactacacacgcccagacctgccttccggggacagccagccctcgctggaccagaccatggccgcggccttcggtaagaggctgcccgacgcggcgccagtgtgcagagtggtctattagggccgctcagtccggagccccggcggcacgggcccggccctgcaccagggtgatgcacctgctgctctctgcacggccagcacagcacggtccagtgtcccccacgtcgggaccacggccccccctggagcagcggatctgcccgcgaaggctctgccggggccgcccgccggccgggttggggcccatcccgcagcacagcgcccgctcgcggtggaggttgtgggtgcgatgattagtgtctcatttatttctaggtgcacctggtataatctcagcctctccgtatgcaggagctggtttccctcccacctttgccattcctcaagctgcaggtattcaaacgcttggtcttggttccccagcgactgcatgcccacaccaccttcccaggcagctccgcatccacggcggcagcctgggcggactgggcactcgagtgccaggtcaggggcccttcccgggagaggggacgccacgtccacagagcaggcttggccagggcagtggtaggacagggctgtggaggcccacgtgtggacggcgccagccagaagccgctaatcgcacagtctttgtggtctggttcccttcaagcacctccgggcgagcgcgaggaccgccagtggttggagggatgtctctagtagttgaatttgagtggcctggtaagcgcgcggcccgaggctgagcagcatgaccggcggcgggacgtgtgtgcgtggccgtcctcccgcttcccttctgggaccggggcccggccgccccagtgctggccgtggacggagcagcgcgtgccgcccggcggccgcctcgtctgcatggaggcatgggcgcgcatgcgcggtggtcccgggacgggccctgcttgccgagggccccgtgtctgtgctggcggagccggagcttttctgtgctgtgggcggggcgcatgcagatgagcccaggcccggcccggcccgtgctgtgaggagaggcgggcgctggtgcaggagccggcgggtggctgtgcgggggtcggacgaccccacaggcacccagggtagggccagagccagggccgcctcccgcgcggctcggctcctcggtgcatgaggacggggagcgtcggcctctcccactctgcggtggagtcgggggcgccgccgctcatctcacctcctgcttttccaggcctttccgttccgaacgtccacggcgccctggcccccctggccatcccctcggcggcggcggcagctgcggcggcaggtcggatcgccatcccgggcctggcgggggcaggaaattctgtattgctggtcagcaacctcaacccagaggtacgtgggcttttcctccgcgccgccgttcctcccggaagagcgagcaggggatgttgtgctcgggctcgggtcccggagcagcgccgcccctcgctgtgtctgcgcctctgccctggcagcccctctgctgcagcgctgagaccctccttttccaagatggcttgagttttcctcttttcctgaattcacatcttggttcgcgttttctcttgcatgatacgcagaatgaattattttttggttacccaaaggcaatctaaaaattacttggagctgagcagggcgcgcagttcagagccccagcaggccgccctgctggtcagcgggacacacagggttagcgcggcggggtgtgggcagcccctgtggctcgagagcctgtcacttaactggtggatccgttatttcccagaatggttaaattcccctcggggagagcttgggtgaaacaagaggcccaaaatatgtaactcttgatggatgggatcctcctggaatcagcatttcaggctgttctgtttcccggcaactcctgtgacttcctcaaggcggtgcccagcctggccctccctcgcgggggcccaggacgcactccctgcctgtgcctcttaccacgagccgggtgctgtccctccagctcctcctgagcgaggccgtgcactaacagactcgatctcccttcacgcctgtaatcgtccttagagactgacttgtgccacagctgcgggagacagcgctgctgcagggcttggctgaggtggccgcgagcggagctgcacgcgcgctcctggggggcgtggttgggatcggcttctgggagcccctccagcagcgctgtctctgccggcatgtgagtgaaggtgcttctcttaccgtgtgcccgtcgaggtaaacctgtaactggaatgtgtgtggagtgtgactgatagaacactacctgattcttatgtatttactgacctgtgtttttttgctactttttttcttttctccccttcccctttccctattttttttcttgccctgatccggaatttctttgccaactgactgcacggtacttctgcttcctgttgttgcttgaaacaaaacaaaaacataaacaaataaaaaacaaaaattccccctcaaaccctgctctccggaaaccaacctgcccttgaatattaacatcctgacaacttcatcatccatcaaccactgcacgcctgcggggactgtcttcctcgtgtggacgattggcaactcgccccccttgacctctccctctcccctgtccctccgctgccttgctctgctgtctctaaagagagtcacaccccaaagcctctttattcttttcggtatgttatcgttcacacttttattaccttgttttcattaattgagatgattttgatgccctgagacgtattatgagtttgcggtttggcactctgatgctccgtggcatccgcctcgttttatggtttgctttcggtttgcgaattttatttggtcccgtagatacgtacgcatggtttatcgccctgcatgcttttcagagttagacctgtcggtggcatatgccaccgtggccacccgctggcagcttacctgtcctggatgctatgactttgctgaacggagctgctcctgttagcgcgccctgtggctgcgagacgcagctccgcagtggccgataaagcaaacccggccgggctgagccgggccttgtgggggtgcgcggggccggggctgacggggagatgggcggggcaggcagcaggagactcaggccccatccctgggcttttgaaggcgtctacggtgacgtgcagcgcgtgaagatcctgttcaataagaaggagaacgccctagtgcagatggcggacggcaaccaggcccagctgggtaagaggccggggcggccccggggtggagggggcaggggcgggggctgcgttccctctcgggcgcctggtcacgcgggtgctgctcccccagccatgagccacctgaacgggcacaagctgcacgggaagcccatccgcatcacgctctcgaagcaccagaacgtgcagctgccccgcgagggccaggaggaccagggcctgaccaaggactacggcaactcacccctgcaccgcttcaagaagccgggctccaagaacttccagaacatattcccgccctcggccacgctgcacctctccaacatcccgtgagtgctgggccggggggctcatggggccgggggcgggcaagggctctgcttggctgtcctaccgcgtcggtgtgtggacttctggcgtttccagagtgcaggtcgggcacctcttaccccaaacctgaagtactgcaaggcctggagcttgagccgagggctgctcaagggagggggtcgttggacactttggaggttttggctcaggggatgctccctgtgagaatgtataatgcacacatttatttatttatattttttgaggcgaagtcttgctttgtcacccaggctggagtacagtggaacgatctcggttcactgcaacctccgcctcttgggttcaagtgattgtgccacctcagcttcccgagtagctgggactgtaggcacccacaaccacacctggctaatttttgtatttttagtagagacggggtttcaccatattggccaggctgatcttgatctcctgaccttgtgatccgcctgcttcggcctcccaaagtgctgggatacaggcgtgagccaccgcgcctagccacatttatttatttatttatttattttacttattttgagatggagtcacgctctgtcgcccaggctggagtgcagtggcgggatctctgcaagttctgcctcctgggttcatgccactctgctgcctcagcctcccgagtagctgggagggactataggtgcccgccagcacgcccggctaattttttgtatttttagtggaaacggggtttcactgtgttagccaggatggtgtcgatctcctgactttgtgatcaacccgcctcggcctcccaaagtgctgggataacaggtgggagccaccacgcctggcctaatgcacacctttataatcccagtactgtggggggccgggcaggggattgcttgaacccaagagttaagaccagcctgagcaataaagtgagacctctgtctctacaggaacaattggttaaaattagctaggagtggtggtgtgcacgtcaggtcccagctcttccggattgggaggttgaggtgggaggttcacttcgcgctcaggaggttgaggttgcagtgagctgtgattgcactgccagactccaggctgggcggcagagtaagaccctgtctgtaaaaattacataatgcaagtatttggaaatgcaggtacatctggccccaagcatgtcaaccagtagtaagccctttgatatttaacacgttacttgtgaaatgttatcagaaaatcaacaaggggcctggcagagtggctcacgcctatcatcccagcactttaggaggccgaggcaggcagatcacctgaggtcaggagttcaagaccaacctggccaacctggtgaaaccccgtctctactaaaaataaaaaaattagcctggcatggtgttgcgtgcctgtaatcccagctacttgggagactgaggcaggagaatcacttgaacctgcaggcggaggttgtggtgagctgagatcacgccactgtgctcccgcctgggcgacagagcaagattccatctggaaaaaaaaaagtggaggagcgtggtactctgtttctgcaaacttgtagatgcctggtttcgttagaagctgcttcagaccattgtggacatgatttgataccccatttctgggctcctaaaaggccctacgccttccccggctactctgaaaactagtctggggaaagcctcgcggacctgactgggcgcccccacccccacgcggccccaggctcacgcctttctcctcccacaggccctcagtctccgaggaggatctcaaggtcctgttttccagcaatgggggcgtcgtcaaaggattcaagttcttccagtgagtatgaggcgggctgtccctggctctccccaggctgccctgcggccggccctgaccccctgtcttgcaggaaggaccgcaagatggcactgatccagatgggctccgtggaggaggcggtccaggccctcattgacctgcacaaccacgacctcggggagaaccaccacctgcgggtctccttctccaagtccaccatctaggggcacaggcccccacggccgggccccctggcgacaacttccatcattccagagaaaagccactttaaaaacagctgaagtgaccttagcagaccagagattttatttttttaaagagaaatcagtttacctgtttttaaaaaaattaaatctagttcaccttgctcaccctgcggtgacagggacagctcaggctcttggtgactgtggcagcgggagttcccggccctccacacccggggccagaccctcggggccatgccttggtggggcctgtgtcgggcgtggggcctgcaggtgggcgccccgaccacgacttggcttccttgtgccttaaaaaacctgccttcctgcagccacacacccacccggggtgtcctggggacccaaggggtgggggggtcacaccagagagaggcagggggcctggccggctcctgcaggatcatgcagctggggcgcggcggccgcggctgcgacaccccaaccccagccctctaatcaagtcacgtgattctcccttcaccccgcccccagggccttcccttctgcccccaggcgggctccccgctgctccagctgcggagctggtcgacataatctctgtattatatactttgcagttgcagacgtctgtgcctagcaatatttccagttgaccaaatattctaatcttttttcatttatatgcaaaagaaatagttttaagtaactttttatagcaagatgatacaatggtatgagtgtaatctaaacttccttgtggtattaccttgtatgctgttacttttattttattccttgtaattaagtcacaggcaggacccagtttccagagagcaggcggggccgcccagtgggtcaggcacagggagccccggtcctatcttagagcccctgagcttcagggaaggggcgggcgtgtcgccgcctctggcatcgcctccggttgccttacaccacgccttcacctgcagtcgcctagaaaacttgctctcaaacttcagggttttttcttccttcaaattttggaccaaagtctcatttctgtgttttgcctgcctctgatgctgggacccggaaggcgggcgctcctcctgtcttctctgtgctctttctaccgcccccgcgtcctgtcccgggggctctcctaggatcccctttccgtaaaagcgtgtaacaagggtgtaaatatttataattttttatacctgttgtgagacccgaggggcggcggcgcggttttttatggtgacacaaatgtatattttgctaacagcaattccaggctcagtattgtgaccgcggagccacaggggaccccacgcacattccgttgccttacccgatggcttgtgacgcggagagaaccgattaaaaccgtttgagaaactcctcccttgtctagccctgtgttcgctgtggacgctgtagaggcaggttggccagtctgtacctggacttcgaataaatcttctgtatcctcgctccgttccgccttcgttgctggcttcctctttctgtgccgcgggcggccccgactcccgcccgggtgtccgccgaagcgccaggtggggccacgggcggggtgggcccggccctgagagtgggggtccctgcgtggggcgtgcgggccagagcgaatgaccaagagccggacctcggtttctgccgctttattgagcatccgcgcggccgcccgcgccctcggcccgccccccgcccgcccccggccccgccgcgctagtcggggaagcggcgcgcctgcatcttgcggaagtagccctcggcctccgcctccgcctcgcgctccgccagccccaggccgcccgcgtggcgccgcagcgtggactcccggctgcccgggcccagcgccccatcggccgcgcccagcgggggcagcccctcgcccgtggccaggttgacggtggccacggccccgaaccgcggctcctcctggtccacgtcgctgaccgacgagccgggggacacgcccgggggcttggccccgccgcggaagccgcgcgccaggtcccccagctcgtagccgccgtcggcctccgccttggccccgccgcccgccgccttccactcccagggcgcgccgccggccgacaggtgcaccacggggtggtgcggcgcgtgcgcgtcgatggtcacgatgctggcggagtcgcggtccgacggcgaccggtactgcgaggagtcggagctggcgctggacgacgacgccgtctcggccgccttggggcccggcgcgcccggagccttggacatggcgatgacctgcagcagccgcggagggttggccactgccgccccgctcttctcggccatcatgagccacttggcgcgcaccccggcgccgcttttgggggacaggccggcccccgtctgcgcgccctcctccgggatgtgcaccagcggcggcggccccgcgcgcggtgggaggatgacccgaggccccagccccggccgcgcctgcacggtggggtagagcgagggcggggccgggccctcgtcctcctcctcttcctcctcctcctcttcctcttcgtcctcctcctcttcctcctcctccgccatgggttcggcgggcgcgcgcaggtgcccggggctggcgtcgtcgggcagccccaggccgcggccctccaggctcttctgcttccactcgctgatgagctgcttggagcgcgaggcgtccagcggcacgtggatggtcatgtggcggcgcgcggggtcgtccagcgagggcgcgctggccctgggcagcgtgtggctgaaggtcaccatgttctccttggccatggggctgcgcggggccagcaggtccacgtccacgctggcgcgcttcaggctgcccagcgaggtcttctcgcgggccacgggccggggcgcgccctccagccgccctgggggccccagctcgtcggtgctcaccgacttattctgctgataaaccgagtcgtggccccgctgcgtcagggcccgcagcgcgtccttggcgggggccggggccgcgggcttctctgcaggtggggcctggaagttgcccaccgcgtggcaggcctgtcggggagaggggtctgggggtgaggcctggggctcagagggctcctcccctgtgatcgttggacttgccgcggggggctctggaccgggggtgggggctggcaagctcttgtccagtgggaccggttcccaccccaaggttgctgacccagcacatcgggcaggtctgagcccacgggattctgactccagcattccaagggcgagacacttcagcacccatgccttccccctaagaccccttcgggacccacgtctatcccctcagacaccctgggcacccgtgcctcccccctagagcccctgggcacccatgcctcccccctcagaccctctgggccagccccccgccagaacccctgggaaccatgcctcacccctcaggctaccagcctcccctgtcagaccccctgagcctgcctcccacctcagacccctgggccagcctccccactcaggccctgtgggcactcatgcctcccccctcagatcccctgggaacccatgccgacccccatcagaacctgccactcaccaggtaggcagcgatgcccgccccgatgaggaagccggcatacacgtccacagggtggctgcggtactgcgtgatctgcgtgagcccgcatacgcccgcggcgatggcaaaggcgaagaccaggatgggcttcagcagcttggtggtgtccgagatgaccgagttgaagtacatctggggcatgggggttggggtcagggagggctccccacgggtcagcaagagggcctgggaagggcagtgaggggccggactcaccgacacatagaccgcggcgaaggctgacagcgtggcgtgctgggacgggaaggtcttcctgtaagaggcgtccagcgtgagccccgcccacctggggaccccagctccagtggcctctctgtgtctctgtttccccgcatgttcaccggggcggaagaaggctcccagtcaggggagttgggggtccggctcaccgtgcagacaggatggcgtggatgtcgtggccggagcagatgtcctgcgtgatgtaggggttgacctcgcaggacgtgcccaggagagtgtagttgggcttgcagacggtgagga

SEQ ID NO: 2 - DNA, 3340 bp, Homo sapiens
tgcgggcgtctccgccattttgtgagtctataactcggagccgttgggtcggttcctgctattccggcgcctccactccgtcccccgcgggtctgctctgtgtgccatggacggcattgtcccagatatagccgttggtacaaagcggggatctgacgagcttttctctacttgtgtcactaacggaccgtttatcatgagcagcaactcggcttctgcagcaaacggaaatgacagcaagaagttcaaaggtgacagccgaagtgcaggcgtcccctctagagtgatccacatccggaagctccccatcgacgtcacggagggggaagtcatctccctggggctgccctttgggaaggtcaccaacctcctgatgctgaaggggaaaaaccaggccttcatcgagatgaacacggaggaggctgccaacaccatggtgaactactacacctcggtgacccctgtgctgcgcggccagcccatctacatccagttctccaaccacaaggagctgaagaccgacagctctcccaaccaggcgcgggcccaggcggccctgcaggcggtgaactcggtccagtcggggaacctggccttggctgcctcggcggcggccgtggacgcagggatggcgatggccgggcagagccccgtgctcaggatcatcgtggagaacctcttctaccctgtgaccctggatgtgctgcaccagattttctccaagttcggcacagtgttgaagatcatcaccttcaccaagaacaaccagttccaggccctgctgcagtatgcggaccccgtgagcgcccagcacgccaagctgtcgctggacgggcagaacatctacaacgcctgctgcacgctgcgcatcgacttttccaagctcaccagcctcaacgtcaagtacaacaatgacaagagccgtgactacacacgcccagacctgccttccggggacagccagccctcgctggaccagaccatggccgcggccttcggtgcacctggtataatctcagcctctccgtatgcaggagctggtttccctcccacctttgccattcctcaagctgcaggcctttccgttccgaacgtccacggcgccctggcccccctggccatcccctcggcggcggcggcagctgcggcggcaggtcggatcgccatcccgggcctggcgggggcaggaaattctgtattgctggtcagcaacctcaacccagagagagtcacaccccaaagcctctttattcttttcggcgtctacggtgacgtgcagcgcgtgaagatcctgttcaataagaaggagaacgccctagtgcagatggcggacggcaaccaggcccagctggccatgagccacctgaacgggcacaagctgcacgggaagcccatccgcatcacgctctcgaagcaccagaacgtgcagctgccccgcgagggccaggaggaccagggcctgaccaaggactacggcaactcacccctgcaccgcttcaagaagccgggctccaagaacttccagaacatattcccgccctcggccacgctgcacctctccaacatcccgccctcagtctccgaggaggatctcaaggtcctgttttccagcaatgggggcgtcgtcaaaggattcaagttcttccagaaggaccgcaagatggcactgatccagatgggctccgtggaggaggcggtccaggccctcattgacctgcacaaccacgacctcggggagaaccaccacctgcgggtctccttctccaagtccaccatctaggggcacaggcccccacggccgggccccctggcgacaacttccatcattccagagaaaagccactttaaaaacagctgaagtgaccttagcagaccagagattttatttttttaaagagaaatcagtttacctgtttttaaaaaaattaaatctagttcaccttgctcaccctgcggtgacagggacagctcaggctcttggtgactgtggcagcgggagttcccggccctccacacccggggccagaccctcggggccatgccttggtggggcctgtgtcgggcgtggggcctgcaggtgggcgccccgaccacgacttggcttccttgtgccttaaaaaacctgccttcctgcagccacacacccacccggggtgtcctggggacccaaggggtgggggggtcacaccagagagaggcagggggcctggccggctcctgcaggatcatgcagctggggcgcggcggccgcggctgcgacaccccaaccccagccctctaatcaagtcacgtgattctcccttcaccccgcccccagggccttcccttctgcccccaggcgggctccccgctgctccagctgcggagctggtcgacataatctctgtattatatactttgcagttgcagacgtctgtgcctagcaatatttccagttgaccaaatattctaatcttttttcatttatatgcaaaagaaatagttttaagtaactttttatagcaagatgatacaatggtatgagtgtaatctaaacttccttgtggtattaccttgtatgctgttacttttattttattccttgtaattaagtcacaggcaggacccagtttccagagagcaggcggggccgcccagtgggtcaggcacagggagccccggtcctatcttagagcccctgagcttcagggaaggggcgggcgtgtcgccgcctctggcatcgcctccggttgccttacaccacgccttcacctgcagtcgcctagaaaacttgctctcaaacttcagggttttttcttccttcaaattttggaccaaagtctcatttctgtgttttgcctgcctctgatgctgggacccggaaggcgggcgctcctcctgtcttctctgtgctctttctaccgcccccgcgtcctgtcccgggggctctcctaggatcccctttccgtaaaagcgtgtaacaagggtgtaaatatttataattttttatacctgttgtgagacccgaggggcggcggcgcggttttttatggtgacacaaatgtatattttgctaacagcaattccaggctcagtattgtgaccgcggagccacaggggaccccacgcacattccgttgccttacccgatggcttgtgacgcggagagaaccgattaaaaccgtttgagaaactcctcccttgtctagccctgtgttcgctgtggacgctgtagaggcaggttggccagtctgtacctggacttcgaataaatcttctgtatcctcgctccgttccgccttaaaaaaaaaaaaaaaaaaaaaaaaaaaaaaaaaaaaaaaaaaaaaaaaaaaaaaaaaaa

SEQ ID NO: 3-8 - skipped (6 entries)

SEQ ID NO: 9 - DNA, 23 bp, Artificial sequence
  Primer
aggaaattctgtattgctggtca

SEQ ID NO: 10 - DNA, 25 bp, Artificial sequence
  Primer
ccttcttattgaacaggatcttcac

SEQ ID NO: 11 - DNA, 29 bp, Artificial sequence
  Synthetic oligonucleotide
agtcacaccccaaagcctctttattcttt

SEQ ID NO: 12-20 - skipped (9 entries)

SEQ ID NO: 21 - DNA, 20 bp, Artificial sequence
  Synthetic oligonucleotide
gttacacgcttttacggaaa

SEQ ID NO: 22 - DNA, 20 bp, Artificial sequence
  Synthetic oligonucleotide
acattcatttttctcttgcc

SEQ ID NO: 23 - DNA, 20 bp, Artificial sequence
  Synthetic oligonucleotide
ccacttttttttttccagat

SEQ ID NO: 24 - DNA, 20 bp, Artificial sequence
  Synthetic oligonucleotide
tggctcttttcctatcaaaa

SEQ ID NO: 25 - DNA, 20 bp, Artificial sequence
  Synthetic oligonucleotide
accttgagatcctcctcgga

SEQ ID NO: 26 - DNA, 20 bp, Artificial sequence
  Synthetic oligonucleotide
ttctcttgccccaaacacca

SEQ ID NO: 27 - DNA, 20 bp, Artificial sequence
  Synthetic oligonucleotide
tggatatcacaccctgtccc

SEQ ID NO: 28 - DNA, 20 bp, Artificial sequence
  Synthetic oligonucleotide
aaagattagaatatttggtc

SEQ ID NO: 29 - DNA, 20 bp, Artificial sequence
  Synthetic oligonucleotide
gccactcaaattcaactact

SEQ ID NO: 30 - DNA, 20 bp, Artificial sequence
  Synthetic oligonucleotide
tccaaccttcgcctcccaca

SEQ ID NO: 31 - DNA, 20 bp, Artificial sequence
  Synthetic oligonucleotide
tgctcctttttccaaattct

SEQ ID NO: 32 - DNA, 20 bp, Artificial sequence
  Synthetic oligonucleotide
gcggctccatcacttctgac

SEQ ID NO: 33 - DNA, 20 bp, Artificial sequence
  Synthetic oligonucleotide
cggcacgattccagcctcta

SEQ ID NO: 34 - DNA, 20 bp, Artificial sequence
  Synthetic oligonucleotide
tctgacctgtccattttctc

SEQ ID NO: 35 - DNA, 20 bp, Artificial sequence
  Synthetic oligonucleotide
aaacactgctcctttttcca

SEQ ID NO: 36 - DNA, 20 bp, Artificial sequence
  Synthetic oligonucleotide
tttagattacactcatacca

SEQ ID NO: 37 - DNA, 20 bp, Artificial sequence
  Synthetic oligonucleotide
aagttacttaaaactatttc

SEQ ID NO: 38 - DNA, 20 bp, Artificial sequence
  Synthetic oligonucleotide
gataacatttcacaagtaac

SEQ ID NO: 39 - DNA, 20 bp, Artificial sequence
  Synthetic oligonucleotide
cctcagttactgatcaaccc

SEQ ID NO: 40 - DNA, 20 bp, Artificial sequence
  Synthetic oligonucleotide
acccccccaccccttgggtc

SEQ ID NO: 41 - DNA, 20 bp, Artificial sequence
  Synthetic oligonucleotide
aatatttacacccttgttac

SEQ ID NO: 42 - DNA, 20 bp, Artificial sequence
  Synthetic oligonucleotide
atgtgtgcattatacattct

SEQ ID NO: 43 - DNA, 20 bp, Artificial sequence
  Synthetic oligonucleotide
atgatcttcaacactgtgcc

SEQ ID NO: 44 - DNA, 20 bp, Artificial sequence
  Synthetic oligonucleotide
aaattataaatatttacacc

SEQ ID NO: 45 - DNA, 20 bp, Artificial sequence
  Synthetic oligonucleotide
gagatgacttccccctccgt

SEQ ID NO: 46 - DNA, 20 bp, Artificial sequence
  Synthetic oligonucleotide
caggtaaactgatttctctt

SEQ ID NO: 47 - DNA, 20 bp, Artificial sequence
  Synthetic oligonucleotide
ccagaaacaatctattcact

SEQ ID NO: 48 - DNA, 20 bp, Artificial sequence
  Synthetic oligonucleotide
ttgctgttagcaaaatatac

SEQ ID NO: 49 - DNA, 20 bp, Artificial sequence
  Synthetic oligonucleotide
acagaaatgagactttggtc

SEQ ID NO: 50 - DNA, 20 bp, Artificial sequence
  Synthetic oligonucleotide
gctaattttaaccaattgtt

SEQ ID NO: 51 - DNA, 20 bp, Artificial sequence
  Synthetic oligonucleotide
tacctctccctcctcccaca

SEQ ID NO: 52 - DNA, 20 bp, Artificial sequence
  Synthetic oligonucleotide
atgcgaaacatctccagcct

SEQ ID NO: 53 - DNA, 20 bp, Artificial sequence
  Synthetic oligonucleotide
tgaacaggatcttcacgcgc

SEQ ID NO: 54 - DNA, 20 bp, Artificial sequence
  Synthetic oligonucleotide
gctgaccagcaatacagaat

SEQ ID NO: 55 - DNA, 20 bp, Artificial sequence
  Synthetic oligonucleotide
ctgtccattttctctccaga

SEQ ID NO: 56 - DNA, 20 bp, Artificial sequence
  Synthetic oligonucleotide
tggcagcctcctccgtgttc

SEQ ID NO: 57 - DNA, 20 bp, Artificial sequence
  Synthetic oligonucleotide
atacagaatttcctgccccc

SEQ ID NO: 58 - DNA, 20 bp, Artificial sequence
  Synthetic oligonucleotide
ttggatcaacttctaagaat

SEQ ID NO: 59 - DNA, 20 bp, Artificial sequence
  Synthetic oligonucleotide
tgcatttccaaatacttgca

SEQ ID NO: 60 - DNA, 20 bp, Artificial sequence
  Synthetic oligonucleotide
gtgtccaacgaccccctccc

SEQ ID NO: 61 - DNA, 20 bp, Artificial sequence
  Synthetic oligonucleotide
tctatccaggtcccctccac

SEQ ID NO: 62 - DNA, 20 bp, Artificial sequence
  Synthetic oligonucleotide
gtttcacccaagctctcccc

SEQ ID NO: 63 - DNA, 20 bp, Artificial sequence
  Synthetic oligonucleotide
tatcaaatcatgtccacaat

SEQ ID NO: 64 - DNA, 20 bp, Artificial sequence
  Synthetic oligonucleotide
gccctggtcctcctggccct

SEQ ID NO: 65 - DNA, 20 bp, Artificial sequence
  Synthetic oligonucleotide
ggtgtctttaaccccacact

SEQ ID NO: 66 - DNA, 20 bp, Artificial sequence
  Synthetic oligonucleotide
gtgacttaattacaaggaat

SEQ ID NO: 67 - DNA, 20 bp, Artificial sequence
  Synthetic oligonucleotide
ttaaaactatttcttttgca

SEQ ID NO: 68 - DNA, 20 bp, Artificial sequence
  Synthetic oligonucleotide
ctccacacacattccagtta

SEQ ID NO: 69 - DNA, 20 bp, Artificial sequence
  Synthetic oligonucleotide
aacaaaggagcctccatctc

SEQ ID NO: 70 - DNA, 20 bp, Artificial sequence
  Synthetic oligonucleotide
gctgcaagattcgcacaccc

SEQ ID NO: 71 - DNA, 20 bp, Artificial sequence
  Synthetic oligonucleotide
cgccccttccctgaagctca

SEQ ID NO: 72 - DNA, 20 bp, Artificial sequence
  Synthetic oligonucleotide
ccaactccagattccagacc

SEQ ID NO: 73 - DNA, 20 bp, Artificial sequence
  Synthetic oligonucleotide
atcagtgccatcttgcggtc

SEQ ID NO: 74 - DNA, 20 bp, Artificial sequence
  Synthetic oligonucleotide
tatcagtcacactccacaca

SEQ ID NO: 75 - DNA, 20 bp, Artificial sequence
  Synthetic oligonucleotide
tgccacagtcaccaagagcc

SEQ ID NO: 76 - DNA, 20 bp, Artificial sequence
  Synthetic oligonucleotide
atttgtgtcaccataaaaaa

SEQ ID NO: 77 - DNA, 20 bp, Artificial sequence
  Synthetic oligonucleotide
ctgcctccctcactctcgca

SEQ ID NO: 78 - DNA, 20 bp, Artificial sequence
  Synthetic oligonucleotide
cctgaagccctaatctcatt

SEQ ID NO: 79 - DNA, 20 bp, Artificial sequence
  Synthetic oligonucleotide
aggaccttcttcttctccag

SEQ ID NO: 80 - DNA, 20 bp, Artificial sequence
  Synthetic oligonucleotide
cacgctcctccacttttttt

SEQ ID NO: 81 - DNA, 20 bp, Artificial sequence
  Synthetic oligonucleotide
gtccacagcgaacacagggc

SEQ ID NO: 82 - DNA, 20 bp, Artificial sequence
  Synthetic oligonucleotide
acagaagatttattcgaagt

SEQ ID NO: 83 - DNA, 20 bp, Artificial sequence
  Synthetic oligonucleotide
tacactcataccattgtatc

SEQ ID NO: 84 - DNA, 20 bp, Artificial sequence
  Synthetic oligonucleotide
ccattgtatcatcttgctat

SEQ ID NO: 85 - DNA, 20 bp, Artificial sequence
  Synthetic oligonucleotide
atcattaaccaactacccca

SEQ ID NO: 86 - DNA, 20 bp, Artificial sequence
  Synthetic oligonucleotide
tcagctgtttttaaagtggc

SEQ ID NO: 87 - DNA, 20 bp, Artificial sequence
  Synthetic oligonucleotide
tgcaaagtatataatacaga

SEQ ID NO: 88 - DNA, 20 bp, Artificial sequence
  Synthetic oligonucleotide
ccatacccacacccacactt

SEQ ID NO: 89 - DNA, 20 bp, Artificial sequence
  Synthetic oligonucleotide
cgcgaagtgaacctcccacc

SEQ ID NO: 90 - DNA, 20 bp, Artificial sequence
  Synthetic oligonucleotide
agacactaatcatcgcaccc

SEQ ID NO: 91 - DNA, 20 bp, Artificial sequence
  Synthetic oligonucleotide
aacaatgttcgaaaccccat

SEQ ID NO: 92 - DNA, 20 bp, Artificial sequence
  Synthetic oligonucleotide
ctgtcccccctcagacaacc

SEQ ID NO: 93 - DNA, 20 bp, Artificial sequence
  Synthetic oligonucleotide
aggccgcctcccctccaccc

SEQ ID NO: 94 - DNA, 20 bp, Artificial sequence
  Synthetic oligonucleotide
ggttctgaccccctttcctc

SEQ ID NO: 95 - DNA, 20 bp, Artificial sequence
  Synthetic oligonucleotide
catcaaaatcatctcaatta

SEQ ID NO: 96 - DNA, 20 bp, Artificial sequence
  Synthetic oligonucleotide
cccctgcctctctctggtgt

SEQ ID NO: 97 - DNA, 20 bp, Artificial sequence
  Synthetic oligonucleotide
tgctgctcatgataaacggt

SEQ ID NO: 98 - DNA, 20 bp, Artificial sequence
  Synthetic oligonucleotide
cctttgaacttcttgctgtc

SEQ ID NO: 99 - DNA, 20 bp, Artificial sequence
  Synthetic oligonucleotide
agagcaagttttctaggcga

SEQ ID NO: 100 - DNA, 20 bp, Artificial sequence
  Synthetic oligonucleotide
aaagtggcttttctctggaa

SEQ ID NO: 101 - DNA, 20 bp, Artificial sequence
  Synthetic oligonucleotide
cattcatttttctcttgccc

SEQ ID NO: 102 - DNA, 20 bp, Artificial sequence
  Synthetic oligonucleotide
tgtccattttctctccagaa

SEQ ID NO: 103 - DNA, 20 bp, Artificial sequence
  Synthetic oligonucleotide
tctgaagcagcttctaacga

SEQ ID NO: 104 - DNA, 20 bp, Artificial sequence
  Synthetic oligonucleotide
acgctcctccactttttttt

SEQ ID NO: 105 - DNA, 20 bp, Artificial sequence
  Synthetic oligonucleotide
ttagattacactcataccat

SEQ ID NO: 106 - DNA, 20 bp, Artificial sequence
  Synthetic oligonucleotide
cccctcctctcctgttttcc

SEQ ID NO: 107 - DNA, 20 bp, Artificial sequence
  Synthetic oligonucleotide
gctggtcttaactcttgggt

SEQ ID NO: 108 - DNA, 20 bp, Artificial sequence
  Synthetic oligonucleotide
gctgttagcaaaatatacat

SEQ ID NO: 109 - DNA, 20 bp, Artificial sequence
  Synthetic oligonucleotide
ttgccgtccgccatctgcac

SEQ ID NO: 110 - DNA, 20 bp, Artificial sequence
  Synthetic oligonucleotide
tgcgaaacatctccagcctc

SEQ ID NO: 111 - DNA, 20 bp, Artificial sequence
  Synthetic oligonucleotide
ctggcattgcccacaactgt

SEQ ID NO: 112 - DNA, 20 bp, Artificial sequence
  Synthetic oligonucleotide
cttgagatcctcctcggaga

SEQ ID NO: 113 - DNA, 20 bp, Artificial sequence
  Synthetic oligonucleotide
cggctccatcacttctgacc

SEQ ID NO: 114 - DNA, 20 bp, Artificial sequence
  Synthetic oligonucleotide
aacactgctcctttttccaa

SEQ ID NO: 115 - DNA, 20 bp, Artificial sequence
  Synthetic oligonucleotide
ttcctgtctgctctcccctc

SEQ ID NO: 116 - DNA, 20 bp, Artificial sequence
  Synthetic oligonucleotide
cctgcaggccccacgcccga

SEQ ID NO: 117 - DNA, 20 bp, Artificial sequence
  Synthetic oligonucleotide
tataatacagagattatgtc

SEQ ID NO: 118 - DNA, 20 bp, Artificial sequence
  Synthetic oligonucleotide
aattataaatatttacaccc

SEQ ID NO: 119 - DNA, 20 bp, Artificial sequence
  Synthetic oligonucleotide
ttcagctccactcctgccct

SEQ ID NO: 120 - DNA, 20 bp, Artificial sequence
  Synthetic oligonucleotide
acagaatttcctgcccccgc

SEQ ID NO: 121 - DNA, 20 bp, Artificial sequence
  Synthetic oligonucleotide
aatcatcgcacccacaacct

SEQ ID NO: 122 - DNA, 20 bp, Artificial sequence
  Synthetic oligonucleotide
gtgtgactctctttagagac

SEQ ID NO: 123 - DNA, 20 bp, Artificial sequence
  Synthetic oligonucleotide
ggatatcacaccctgtcccc

SEQ ID NO: 124 - DNA, 20 bp, Artificial sequence
  Synthetic oligonucleotide
ccttgttgattttctgataa

SEQ ID NO: 125 - DNA, 20 bp, Artificial sequence
  Synthetic oligonucleotide
accaacggctatatctggga

SEQ ID NO: 126 - DNA, 20 bp, Artificial sequence
  Synthetic oligonucleotide
gcacacagagccctcgctgc

SEQ ID NO: 127 - DNA, 20 bp, Artificial sequence
  Synthetic oligonucleotide
atatttacacccttgttaca

SEQ ID NO: 128 - DNA, 20 bp, Artificial sequence
  Synthetic oligonucleotide
ttcgaaaccccatctttacc

SEQ ID NO: 129 - DNA, 20 bp, Artificial sequence
  Synthetic oligonucleotide
ctgcaagattcgcacacccc

SEQ ID NO: 130 - DNA, 20 bp, Artificial sequence
  Synthetic oligonucleotide
ggtaaactgatttctcttta

SEQ ID NO: 131 - DNA, 20 bp, Artificial sequence
  Synthetic oligonucleotide
tgtgtgcattatacattctc

SEQ ID NO: 132 - DNA, 20 bp, Artificial sequence
  Synthetic oligonucleotide
gaccttcttcttctccaggt

SEQ ID NO: 133 - DNA, 20 bp, Artificial sequence
  Synthetic oligonucleotide
acccacacccacacttccac

SEQ ID NO: 134 - DNA, 20 bp, Artificial sequence
  Synthetic oligonucleotide
gaggagtttctcaaacggtt

SEQ ID NO: 135 - DNA, 20 bp, Artificial sequence
  Synthetic oligonucleotide
ccacacacattccagttaca

SEQ ID NO: 136 - DNA, 20 bp, Artificial sequence
  Synthetic oligonucleotide
agttacttaaaactatttct

SEQ ID NO: 137 - DNA, 20 bp, Artificial sequence
  Synthetic oligonucleotide
gtgcagcacatccagggtca

SEQ ID NO: 138 - DNA, 20 bp, Artificial sequence
  Synthetic oligonucleotide
gcactagactgatcctaggc

SEQ ID NO: 139 - DNA, 20 bp, Artificial sequence
  Synthetic oligonucleotide
taaaactatttcttttgcat

SEQ ID NO: 140 - DNA, 20 bp, Artificial sequence
  Synthetic oligonucleotide
ctgctcatgataaacggtcc

SEQ ID NO: 141 - DNA, 20 bp, Artificial sequence
  Synthetic oligonucleotide
tttgaacttcttgctgtcat

SEQ ID NO: 142 - DNA, 20 bp, Artificial sequence
  Synthetic oligonucleotide
cagaagatttattcgaagtc

SEQ ID NO: 143 - DNA, 20 bp, Artificial sequence
  Synthetic oligonucleotide
aaacaatctattcactggtt

SEQ ID NO: 144 - DNA, 20 bp, Artificial sequence
  Synthetic oligonucleotide
gccccgcctgctctctggaa

SEQ ID NO: 145 - DNA, 20 bp, Artificial sequence
  Synthetic oligonucleotide
atcatcttgctataaaaagt

SEQ ID NO: 146 - DNA, 20 bp, Artificial sequence
  Synthetic oligonucleotide
ttcacccaagctctccccga

SEQ ID NO: 147 - DNA, 20 bp, Artificial sequence
  Synthetic oligonucleotide
atccaggtcccctccacagc

SEQ ID NO: 148 - DNA, 20 bp, Artificial sequence
  Synthetic oligonucleotide
tggtgaccttcccaaagggc

SEQ ID NO: 149 - DNA, 20 bp, Artificial sequence
  Synthetic oligonucleotide
ctgaccagcaatacagaatt

SEQ ID NO: 150 - DNA, 20 bp, Artificial sequence
  Synthetic oligonucleotide
actcaaattcaactactaga

SEQ ID NO: 151 - DNA, 20 bp, Artificial sequence
  Synthetic oligonucleotide
tcttgcggtccttcctgcaa

SEQ ID NO: 152 - DNA, 20 bp, Artificial sequence
  Synthetic oligonucleotide
gtccaacgaccccctccctt

SEQ ID NO: 153 - DNA, 20 bp, Artificial sequence
  Synthetic oligonucleotide
gccacagtcaccaagagcct

SEQ ID NO: 154 - DNA, 20 bp, Artificial sequence
  Synthetic oligonucleotide
aacaggttttatctccagga

SEQ ID NO: 155 - DNA, 20 bp, Artificial sequence
  Synthetic oligonucleotide
aacaccacctttgacagttt

SEQ ID NO: 156 - DNA, 20 bp, Artificial sequence
  Synthetic oligonucleotide
ggctcttttcctatcaaaac

SEQ ID NO: 157 - DNA, 20 bp, Artificial sequence
  Synthetic oligonucleotide
gcctccccaggaccttcttc

SEQ ID NO: 158 - DNA, 20 bp, Artificial sequence
  Synthetic oligonucleotide
gatgacttccccctccgtga

SEQ ID NO: 159 - DNA, 20 bp, Artificial sequence
  Synthetic oligonucleotide
gttctgaccccctttcctca

SEQ ID NO: 160 - DNA, 20 bp, Artificial sequence
  Synthetic oligonucleotide
aagattagaatatttggtca

SEQ ID NO: 161 - DNA, 20 bp, Artificial sequence
  Synthetic oligonucleotide
ccgacccgactctccctcct

SEQ ID NO: 162 - DNA, 20 bp, Artificial sequence
  Synthetic oligonucleotide
atgtacctgcatttccaaat

SEQ ID NO: 163 - DNA, 20 bp, Artificial sequence
  Synthetic oligonucleotide
cctttttccaaattctggaa

SEQ ID NO: 164 - DNA, 20 bp, Artificial sequence
  Synthetic oligonucleotide
ctgcaagccagacacatcta

SEQ ID NO: 165 - DNA, 20 bp, Artificial sequence
  Synthetic oligonucleotide
gcatttccaaatacttgcat

SEQ ID NO: 166 - DNA, 20 bp, Artificial sequence
  Synthetic oligonucleotide
acaacccccagctccacaca

SEQ ID NO: 167 - DNA, 20 bp, Artificial sequence
  Synthetic oligonucleotide
gaacaggatcttcacgcgct

SEQ ID NO: 168 - DNA, 20 bp, Artificial sequence
  Synthetic oligonucleotide
agtaacagcatacaaggtaa

SEQ ID NO: 169 - DNA, 20 bp, Artificial sequence
  Synthetic oligonucleotide
gcgaagtgaacctcccacct

SEQ ID NO: 170 - DNA, 20 bp, Artificial sequence
  Synthetic oligonucleotide
acactcataccattgtatca

SEQ ID NO: 171 - DNA, 20 bp, Artificial sequence
  Synthetic oligonucleotide
atcagtcacactccacacac

SEQ ID NO: 172 - DNA, 20 bp, Artificial sequence
  Synthetic oligonucleotide
gtgtcaccataaaaaaccgc

SEQ ID NO: 173 - DNA, 20 bp, Artificial sequence
  Synthetic oligonucleotide
aggagcctccatctcccgta

SEQ ID NO: 174 - DNA, 20 bp, Artificial sequence
  Synthetic oligonucleotide
ctgacctgtccattttctct

SEQ ID NO: 175 - DNA, 20 bp, Artificial sequence
  Synthetic oligonucleotide
ctggtgtgacccccccaccc

SEQ ID NO: 176 - DNA, 20 bp, Artificial sequence
  Synthetic oligonucleotide
ttacacccttgttacacgct

SEQ ID NO: 177 - DNA, 20 bp, Artificial sequence
  Synthetic oligonucleotide
aagtggcttttctctggaat

SEQ ID NO: 178 - DNA, 20 bp, Artificial sequence
  Synthetic oligonucleotide
attcatttttctcttgcccc

SEQ ID NO: 179 - DNA, 20 bp, Artificial sequence
  Synthetic oligonucleotide
tcttgctgtcatttccgttt

SEQ ID NO: 180 - DNA, 20 bp, Artificial sequence
  Synthetic oligonucleotide
gctcttttcctatcaaaacc

SEQ ID NO: 181 - DNA, 20 bp, Artificial sequence
  Synthetic oligonucleotide
tgcagcacatccagggtcac

SEQ ID NO: 182 - DNA, 20 bp, Artificial sequence
  Synthetic oligonucleotide
ggtgaccttcccaaagggca

SEQ ID NO: 183 - DNA, 20 bp, Artificial sequence
  Synthetic oligonucleotide
cgctcctccacttttttttt

SEQ ID NO: 184 - DNA, 20 bp, Artificial sequence
  Synthetic oligonucleotide
ccaggtcccctccacagcca

SEQ ID NO: 185 - DNA, 20 bp, Artificial sequence
  Synthetic oligonucleotide
cgtggcacattcatttttct

SEQ ID NO: 186 - DNA, 20 bp, Artificial sequence
  Synthetic oligonucleotide
gatgaaagtacctctccctc

SEQ ID NO: 187 - DNA, 20 bp, Artificial sequence
  Synthetic oligonucleotide
atgcccatcctagccactca

SEQ ID NO: 188 - DNA, 20 bp, Artificial sequence
  Synthetic oligonucleotide
tccccaggaccttcttcttc

SEQ ID NO: 189 - DNA, 20 bp, Artificial sequence
  Synthetic oligonucleotide
ctggaagaacttgaatcctt

SEQ ID NO: 190 - DNA, 20 bp, Artificial sequence
  Synthetic oligonucleotide
ccgggtttgctttatcggcc

SEQ ID NO: 191 - DNA, 20 bp, Artificial sequence
  Synthetic oligonucleotide
gccgtccgccatctgcacta

SEQ ID NO: 192 - DNA, 20 bp, Artificial sequence
  Synthetic oligonucleotide
atgacttccccctccgtgac

SEQ ID NO: 193 - DNA, 20 bp, Artificial sequence
  Synthetic oligonucleotide
acactgctcctttttccaaa

SEQ ID NO: 194 - DNA, 20 bp, Artificial sequence
  Synthetic oligonucleotide
tgtacctgcatttccaaata

SEQ ID NO: 195 - DNA, 20 bp, Artificial sequence
  Synthetic oligonucleotide
actggaaatattgctaggca

SEQ ID NO: 196 - DNA, 20 bp, Artificial sequence
  Synthetic oligonucleotide
gtaacagcatacaaggtaat

SEQ ID NO: 197 - DNA, 20 bp, Artificial sequence
  Synthetic oligonucleotide
atttccaaatacttgcatta

SEQ ID NO: 198 - DNA, 20 bp, Artificial sequence
  Synthetic oligonucleotide
aacaatctattcactggtta

SEQ ID NO: 199 - DNA, 20 bp, Artificial sequence
  Synthetic oligonucleotide
ccccacgcccgacacaggcc

SEQ ID NO: 200 - DNA, 20 bp, Artificial sequence
  Synthetic oligonucleotide
tgacctgtccattttctctc

SEQ ID NO: 201 - DNA, 20 bp, Artificial sequence
  Synthetic oligonucleotide
gtgtgcattatacattctca

SEQ ID NO: 202 - DNA, 20 bp, Artificial sequence
  Synthetic oligonucleotide
gtgaactagatttaattttt

SEQ ID NO: 203 - DNA, 20 bp, Artificial sequence
  Synthetic oligonucleotide
cgacacgcccgccccttccc

SEQ ID NO: 204 - DNA, 20 bp, Artificial sequence
  Synthetic oligonucleotide
attataaatatttacaccct

SEQ ID NO: 205 - DNA, 20 bp, Artificial sequence
  Synthetic oligonucleotide
tggtgtgacccccccacccc

SEQ ID NO: 206 - DNA, 20 bp, Artificial sequence
  Synthetic oligonucleotide
agtcacactccacacacatt

SEQ ID NO: 207 - DNA, 20 bp, Artificial sequence
  Synthetic oligonucleotide
tagattacactcataccatt

SEQ ID NO: 208 - DNA, 20 bp, Artificial sequence
  Synthetic oligonucleotide
agtaccacgctcctccactt

SEQ ID NO: 209 - DNA, 20 bp, Artificial sequence
  Synthetic oligonucleotide
acatctccagcctctcccgc

SEQ ID NO: 210 - DNA, 20 bp, Artificial sequence
  Synthetic oligonucleotide
gagcagctcagattccagac

SEQ ID NO: 211 - DNA, 20 bp, Artificial sequence
  Synthetic oligonucleotide
aagcaccttcactcacatgc

SEQ ID NO: 212 - DNA, 20 bp, Artificial sequence
  Synthetic oligonucleotide
gagcctccatctcccgtatc

SEQ ID NO: 213 - DNA, 20 bp, Artificial sequence
  Synthetic oligonucleotide
gctgactgaccagaaaccaa

SEQ ID NO: 214 - DNA, 20 bp, Artificial sequence
  Synthetic oligonucleotide
ccggccaggccccctgcctc

SEQ ID NO: 215 - DNA, 20 bp, Artificial sequence
  Synthetic oligonucleotide
cgtacactatctccacggcc

SEQ ID NO: 216 - DNA, 20 bp, Artificial sequence
  Synthetic oligonucleotide
tgtgactctctttagagaca

SEQ ID NO: 217 - DNA, 20 bp, Artificial sequence
  Synthetic oligonucleotide
cgacccgactctccctcctc

SEQ ID NO: 218 - DNA, 20 bp, Artificial sequence
  Synthetic oligonucleotide
tgctgctgttctaaccagat

SEQ ID NO: 219 - DNA, 20 bp, Artificial sequence
  Synthetic oligonucleotide
acacaagtagagaaaagctc

SEQ ID NO: 220 - DNA, 20 bp, Artificial sequence
  Synthetic oligonucleotide
atatcacaccctgtcccccc

SEQ ID NO: 221 - DNA, 20 bp, Artificial sequence
  Synthetic oligonucleotide
ttgagatcctcctcggagac

SEQ ID NO: 222 - DNA, 20 bp, Artificial sequence
  Synthetic oligonucleotide
gtcaccataaaaaaccgcgc

SEQ ID NO: 223 - DNA, 20 bp, Artificial sequence
  Synthetic oligonucleotide
accagcaatacagaatttcc

SEQ ID NO: 224 - DNA, 20 bp, Artificial sequence
  Synthetic oligonucleotide
actagactgatcctaggctc

SEQ ID NO: 225 - DNA, 20 bp, Artificial sequence
  Synthetic oligonucleotide
ctggtcttaactcttgggtt

SEQ ID NO: 226 - DNA, 20 bp, Artificial sequence
  Synthetic oligonucleotide
ctgttagcaaaatatacatt

SEQ ID NO: 227 - DNA, 20 bp, Artificial sequence
  Synthetic oligonucleotide
gtaaactgatttctctttaa

SEQ ID NO: 228 - DNA, 20 bp, Artificial sequence
  Synthetic oligonucleotide
tcaaacggttttaatcggtt

SEQ ID NO: 229 - DNA, 20 bp, Artificial sequence
  Synthetic oligonucleotide
gctccaagtaatttttagat

SEQ ID NO: 230 - DNA, 20 bp, Artificial sequence
  Synthetic oligonucleotide
ttctgaccccctttcctcac

SEQ ID NO: 231 - DNA, 20 bp, Artificial sequence
  Synthetic oligonucleotide
cctgtctgctctcccctcca

SEQ ID NO: 232 - DNA, 20 bp, Artificial sequence
  Synthetic oligonucleotide
accttcttcttctccaggtt

SEQ ID NO: 233 - DNA, 20 bp, Artificial sequence
  Synthetic oligonucleotide
tcatcttgctataaaaagtt

SEQ ID NO: 234 - DNA, 20 bp, Artificial sequence
  Synthetic oligonucleotide
gttacttaaaactatttctt

SEQ ID NO: 235 - DNA, 20 bp, Artificial sequence
  Synthetic oligonucleotide
aaaactatttcttttgcata

SEQ ID NO: 236 - DNA, 20 bp, Artificial sequence
  Synthetic oligonucleotide
ggctccatcacttctgacct

SEQ ID NO: 237 - DNA, 20 bp, Artificial sequence
  Synthetic oligonucleotide
gcgcctccactgccttcaca

SEQ ID NO: 238 - DNA, 20 bp, Artificial sequence
  Synthetic oligonucleotide
ccctcctctcctgttttcca

SEQ ID NO: 239 - DNA, 20 bp, Artificial sequence
  Synthetic oligonucleotide
actcataccattgtatcatc

SEQ ID NO: 240 - DNA, 20 bp, Artificial sequence
  Synthetic oligonucleotide
gtccattttctctccagaaa

SEQ ID NO: 241 - DNA, 20 bp, Artificial sequence
  Synthetic oligonucleotide
caccaatcccacaagcactc

SEQ ID NO: 242 - DNA, 20 bp, Artificial sequence
  Synthetic oligonucleotide
acagactggccaacctgcct

SEQ ID NO: 243 - DNA, 20 bp, Artificial sequence
  Synthetic oligonucleotide
ccacacccacacttccacgg

SEQ ID NO: 244 - DNA, 20 bp, Artificial sequence
  Synthetic oligonucleotide
cccccagctccacacacccc

SEQ ID NO: 245 - DNA, 20 bp, Artificial sequence
  Synthetic oligonucleotide
actcctagctaattttaacc

SEQ ID NO: 246 - DNA, 20 bp, Artificial sequence
  Synthetic oligonucleotide
cccgcctgctctctggaaac

SEQ ID NO: 247 - DNA, 20 bp, Artificial sequence
  Synthetic oligonucleotide
ttgattttctgataacattt

SEQ ID NO: 248 - DNA, 20 bp, Artificial sequence
  Synthetic oligonucleotide
agaatttcctgcccccgcca

SEQ ID NO: 249 - DNA, 20 bp, Artificial sequence
  Synthetic oligonucleotide
acgccagaagtccacacacc

SEQ ID NO: 250 - DNA, 20 bp, Artificial sequence
  Synthetic oligonucleotide
actactagagacatccctcc

SEQ ID NO: 251 - DNA, 20 bp, Artificial sequence
  Synthetic oligonucleotide
ccaacggctatatctgggac

SEQ ID NO: 252 - DNA, 20 bp, Artificial sequence
  Synthetic oligonucleotide
gcacccacaacctccaccgc

SEQ ID NO: 253 - DNA, 20 bp, Artificial sequence
  Synthetic oligonucleotide
tacacccttgttacacgctt

SEQ ID NO: 254 - DNA, 20 bp, Artificial sequence
  Synthetic oligonucleotide
gaactagatttaattttttt

SEQ ID NO: 255 - DNA, 20 bp, Artificial sequence
  Synthetic oligonucleotide
ctggaccgagttcaccgcct

SEQ ID NO: 256 - DNA, 20 bp, Artificial sequence
  Synthetic oligonucleotide
tgtgcattatacattctcac

SEQ ID NO: 257 - DNA, 20 bp, Artificial sequence
  Synthetic oligonucleotide
ctagactgatcctaggctca

SEQ ID NO: 258 - DNA, 20 bp, Artificial sequence
  Synthetic oligonucleotide
tgcctgtgacttaattacaa

SEQ ID NO: 259 - DNA, 20 bp, Artificial sequence
  Synthetic oligonucleotide
atgaaagtacctctccctcc

SEQ ID NO: 260 - DNA, 20 bp, Artificial sequence
  Synthetic oligonucleotide
ccagcaatacagaatttcct

SEQ ID NO: 261 - DNA, 20 bp, Artificial sequence
  Synthetic oligonucleotide
tcactggttatttctatgta

SEQ ID NO: 262 - DNA, 20 bp, Artificial sequence
  Synthetic oligonucleotide
aaggaagtttagattacact

SEQ ID NO: 263 - DNA, 20 bp, Artificial sequence
  Synthetic oligonucleotide
gcgaggctttccccagacta

SEQ ID NO: 264 - DNA, 20 bp, Artificial sequence
  Synthetic oligonucleotide
ggtgtgacccccccacccct

SEQ ID NO: 265 - DNA, 20 bp, Artificial sequence
  Synthetic oligonucleotide
ccagggagatgacttccccc

SEQ ID NO: 266 - DNA, 20 bp, Artificial sequence
  Synthetic oligonucleotide
ctggaaatattgctaggcac

SEQ ID NO: 267 - DNA, 20 bp, Artificial sequence
  Synthetic oligonucleotide
actagagacatccctccaac

SEQ ID NO: 268 - DNA, 20 bp, Artificial sequence
  Synthetic oligonucleotide
tcaccataaaaaaccgcgcc

SEQ ID NO: 269 - DNA, 20 bp, Artificial sequence
  Synthetic oligonucleotide
ttataaatatttacaccctt

SEQ ID NO: 270 - DNA, 20 bp, Artificial sequence
  Synthetic oligonucleotide
tccgccatctgcactagggc

SEQ ID NO: 271 - DNA, 20 bp, Artificial sequence
  Synthetic oligonucleotide
gtaccacgctcctccacttt

SEQ ID NO: 272 - DNA, 20 bp, Artificial sequence
  Synthetic oligonucleotide
tatcacaccctgtcccccct

SEQ ID NO: 273 - DNA, 20 bp, Artificial sequence
  Synthetic oligonucleotide
gcggagctgcacttcccacc

SEQ ID NO: 274 - DNA, 20 bp, Artificial sequence
  Synthetic oligonucleotide
atggcgatccgacctgccgc

SEQ ID NO: 275 - DNA, 20 bp, Artificial sequence
  Synthetic oligonucleotide
caacggctatatctgggaca

SEQ ID NO: 276 - DNA, 20 bp, Artificial sequence
  Synthetic oligonucleotide
gtggcacattcatttttctc

SEQ ID NO: 277 - DNA, 20 bp, Artificial sequence
  Synthetic oligonucleotide
tcatttttctcttgccccaa

SEQ ID NO: 278 - DNA, 20 bp, Artificial sequence
  Synthetic oligonucleotide
aggtccctgctatcccccta

SEQ ID NO: 279 - DNA, 20 bp, Artificial sequence
  Synthetic oligonucleotide
gtacactatctccacggcca

SEQ ID NO: 280 - DNA, 20 bp, Artificial sequence
  Synthetic oligonucleotide
tcctccacttttttttttcc

SEQ ID NO: 281 - DNA, 20 bp, Artificial sequence
  Synthetic oligonucleotide
agcaccttcactcacatgcc

SEQ ID NO: 282 - DNA, 20 bp, Artificial sequence
  Synthetic oligonucleotide
gttctatcagtcacactcca

SEQ ID NO: 283 - DNA, 20 bp, Artificial sequence
  Synthetic oligonucleotide
atctccagcctctcccgctt

SEQ ID NO: 284 - DNA, 20 bp, Artificial sequence
  Synthetic oligonucleotide
cggccaggccccctgcctct

SEQ ID NO: 285 - DNA, 20 bp, Artificial sequence
  Synthetic oligonucleotide
ttcggctgtcacctttgaac

SEQ ID NO: 286 - DNA, 20 bp, Artificial sequence
  Synthetic oligonucleotide
ctgctgttctaaccagatgc

SEQ ID NO: 287 - DNA, 20 bp, Artificial sequence
  Synthetic oligonucleotide
gacctgtccattttctctcc

SEQ ID NO: 288 - DNA, 20 bp, Artificial sequence
  Synthetic oligonucleotide
tggtggttctccccgaggtc

SEQ ID NO: 289 - DNA, 20 bp, Artificial sequence
  Synthetic oligonucleotide
tctgaccccctttcctcaca

SEQ ID NO: 290 - DNA, 20 bp, Artificial sequence
  Synthetic oligonucleotide
ccttcttcttctccaggttc

SEQ ID NO: 291 - DNA, 20 bp, Artificial sequence
  Synthetic oligonucleotide
gccagaagtccacacaccga

SEQ ID NO: 292 - DNA, 20 bp, Artificial sequence
  Synthetic oligonucleotide
accagcagaatcattaacca

SEQ ID NO: 293 - DNA, 20 bp, Artificial sequence
  Synthetic oligonucleotide
tccattttctctccagaaac

SEQ ID NO: 294 - DNA, 20 bp, Artificial sequence
  Synthetic oligonucleotide
ctcttttcctatcaaaacca

SEQ ID NO: 295 - DNA, 20 bp, Artificial sequence
  Synthetic oligonucleotide
gcatatgccaccgacaggtc

SEQ ID NO: 296 - DNA, 20 bp, Artificial sequence
  Synthetic oligonucleotide
ctcctagctaattttaacca

SEQ ID NO: 297 - DNA, 20 bp, Artificial sequence
  Synthetic oligonucleotide
caaacggttttaatcggttc

SEQ ID NO: 298 - DNA, 20 bp, Artificial sequence
  Synthetic oligonucleotide
gcttggtcttctcttaaggc

SEQ ID NO: 299 - DNA, 20 bp, Artificial sequence
  Synthetic oligonucleotide
aggtggctcttttcctatca

SEQ ID NO: 300 - DNA, 20 bp, Artificial sequence
  Synthetic oligonucleotide
cctaggtgtctttaacccca

SEQ ID NO: 301 - DNA, 20 bp, Artificial sequence
  Synthetic oligonucleotide
gtctgctctcccctccagct

SEQ ID NO: 302 - DNA, 20 bp, Artificial sequence
  Synthetic oligonucleotide
ccaaatacttgcattatgta

SEQ ID NO: 303 - DNA, 20 bp, Artificial sequence
  Synthetic oligonucleotide
aaggtgatgatcttcaacac

SEQ ID NO: 304 - DNA, 20 bp, Artificial sequence
  Synthetic oligonucleotide
ccccaggaccttcttcttct

SEQ ID NO: 305 - DNA, 20 bp, Artificial sequence
  Synthetic oligonucleotide
ctgttcagtccccaagactt

SEQ ID NO: 306 - DNA, 20 bp, Artificial sequence
  Synthetic oligonucleotide
tccatcacttctgacctgtc

SEQ ID NO: 307 - DNA, 20 bp, Artificial sequence
  Synthetic oligonucleotide
tcttttgcatataaatgaaa

SEQ ID NO: 308 - DNA, 20 bp, Artificial sequence
  Synthetic oligonucleotide
gggcacctatagtccctccc

SEQ ID NO: 309 - DNA, 20 bp, Artificial sequence
  Synthetic oligonucleotide
gctgcgcctcttctgacacc

SEQ ID NO: 310 - DNA, 20 bp, Artificial sequence
  Synthetic oligonucleotide
tgttagcaaaatatacattt

SEQ ID NO: 311 - DNA, 20 bp, Artificial sequence
  Synthetic oligonucleotide
cctctcctgttttccagccc

SEQ ID NO: 312 - DNA, 20 bp, Artificial sequence
  Synthetic oligonucleotide
ttttctgataacatttcaca

SEQ ID NO: 313 - DNA, 20 bp, Artificial sequence
  Synthetic oligonucleotide
tccatctcccgtatcaggat

SEQ ID NO: 314 - DNA, 20 bp, Artificial sequence
  Synthetic oligonucleotide
cactgctcctttttccaaat

SEQ ID NO: 315 - DNA, 20 bp, Artificial sequence
  Synthetic oligonucleotide
aactcccgctgccacagtca

SEQ ID NO: 316 - DNA, 20 bp, Artificial sequence
  Synthetic oligonucleotide
ctccaagtaatttttagatt

SEQ ID NO: 317 - DNA, 20 bp, Artificial sequence
  Synthetic oligonucleotide
cagactggccaacctgcctc

SEQ ID NO: 318 - DNA, 20 bp, Artificial sequence
  Synthetic oligonucleotide
ctcataccattgtatcatct

SEQ ID NO: 319 - DNA, 20 bp, Artificial sequence
  Synthetic oligonucleotide
gaacttgaatcctttgacga

SEQ ID NO: 320 - DNA, 20 bp, Artificial sequence
  Synthetic oligonucleotide
catcttgctataaaaagtta

SEQ ID NO: 321 - DNA, 20 bp, Artificial sequence
  Synthetic oligonucleotide
gacacgcccgccccttccct

SEQ ID NO: 322 - DNA, 20 bp, Artificial sequence
  Synthetic oligonucleotide
ttacttaaaactatttcttt

SEQ ID NO: 323 - DNA, 20 bp, Artificial sequence
  Synthetic oligonucleotide
gtcacactccacacacattc

SEQ ID NO: 324 - DNA, 20 bp, Artificial sequence
  Synthetic oligonucleotide
gattacactcataccattgt

SEQ ID NO: 325 - DNA, 20 bp, Artificial sequence
  Synthetic oligonucleotide
gtacctgcatttccaaatac

SEQ ID NO: 326 - DNA, 20 bp, Artificial sequence
  Synthetic oligonucleotide
cacacccacacttccacggc

SEQ ID NO: 327 - DNA, 20 bp, Artificial sequence
  Synthetic oligonucleotide
ggctggattctatccaggtc

SEQ ID NO: 328 - DNA, 20 bp, Artificial sequence
  Synthetic oligonucleotide
agcagctcagattccagacc

SEQ ID NO: 329 - DNA, 20 bp, Artificial sequence
  Synthetic oligonucleotide
taaactgatttctctttaaa

SEQ ID NO: 330 - DNA, 20 bp, Artificial sequence
  Synthetic oligonucleotide
acacccttgttacacgcttt

SEQ ID NO: 331 - DNA, 20 bp, Artificial sequence
  Synthetic oligonucleotide
ggcacattcatttttctctt

SEQ ID NO: 332 - DNA, 20 bp, Artificial sequence
  Synthetic oligonucleotide
cctccacttttttttttcca

SEQ ID NO: 333 - DNA, 20 bp, Artificial sequence
  Synthetic oligonucleotide
gcccaactccagattccaga

SEQ ID NO: 334 - DNA, 20 bp, Artificial sequence
  Synthetic oligonucleotide
tcggctgtcacctttgaact

SEQ ID NO: 335 - DNA, 20 bp, Artificial sequence
  Synthetic oligonucleotide
gcaactgcaaagtatataat

SEQ ID NO: 336 - DNA, 20 bp, Artificial sequence
  Synthetic oligonucleotide
gccgatcccaaccacgcccc

SEQ ID NO: 337 - DNA, 20 bp, Artificial sequence
  Synthetic oligonucleotide
caatacagaatttcctgccc

SEQ ID NO: 338 - DNA, 20 bp, Artificial sequence
  Synthetic oligonucleotide
acacccacacttccacggca

SEQ ID NO: 339 - DNA, 20 bp, Artificial sequence
  Synthetic oligonucleotide
taccattgtatcatcttgct

SEQ ID NO: 340 - DNA, 20 bp, Artificial sequence
  Synthetic oligonucleotide
gctttccccagactagtttt

SEQ ID NO: 341 - DNA, 20 bp, Artificial sequence
  Synthetic oligonucleotide
gtgtgacccccccacccctt

SEQ ID NO: 342 - DNA, 20 bp, Artificial sequence
  Synthetic oligonucleotide
taggtgtctttaaccccaca

SEQ ID NO: 343 - DNA, 20 bp, Artificial sequence
  Synthetic oligonucleotide
agggagatgacttccccctc

SEQ ID NO: 344 - DNA, 20 bp, Artificial sequence
  Synthetic oligonucleotide
accagtggatatcacaccct

SEQ ID NO: 345 - DNA, 20 bp, Artificial sequence
  Synthetic oligonucleotide
gttggcagcctcctccgtgt

SEQ ID NO: 346 - DNA, 20 bp, Artificial sequence
  Synthetic oligonucleotide
gcagaatcattaaccaacta

SEQ ID NO: 347 - DNA, 20 bp, Artificial sequence
  Synthetic oligonucleotide
acgcccgccccttccctgaa

SEQ ID NO: 348 - DNA, 20 bp, Artificial sequence
  Synthetic oligonucleotide
gctgttctaaccagatgcca

SEQ ID NO: 349 - DNA, 20 bp, Artificial sequence
  Synthetic oligonucleotide
ccattttctctccagaaaca

SEQ ID NO: 350 - DNA, 20 bp, Artificial sequence
  Synthetic oligonucleotide
agcaaaatatacatttgtgt

SEQ ID NO: 351 - DNA, 20 bp, Artificial sequence
  Synthetic oligonucleotide
tacactatctccacggccaa

SEQ ID NO: 352 - DNA, 20 bp, Artificial sequence
  Synthetic oligonucleotide
accaaataaaattcgcaaac

SEQ ID NO: 353 - DNA, 20 bp, Artificial sequence
  Synthetic oligonucleotide
tcacactccacacacattcc

SEQ ID NO: 354 - DNA, 20 bp, Artificial sequence
  Synthetic oligonucleotide
atcacaccctgtcccccctc

SEQ ID NO: 355 - DNA, 20 bp, Artificial sequence
  Synthetic oligonucleotide
cttgaatcctttgacgacgc

SEQ ID NO: 356 - DNA, 20 bp, Artificial sequence
  Synthetic oligonucleotide
actggccaacctgcctctac

SEQ ID NO: 357 - DNA, 20 bp, Artificial sequence
  Synthetic oligonucleotide
tcccgctgccacagtcacca

SEQ ID NO: 358 - DNA, 20 bp, Artificial sequence
  Synthetic oligonucleotide
agtttagattacactcatac

SEQ ID NO: 359 - DNA, 20 bp, Artificial sequence
  Synthetic oligonucleotide
gtgatgatcttcaacactgt

SEQ ID NO: 360 - DNA, 20 bp, Artificial sequence
  Synthetic oligonucleotide
ggtggctcttttcctatcaa

SEQ ID NO: 361 - DNA, 20 bp, Artificial sequence
  Synthetic oligonucleotide
cactggttatttctatgtaa

SEQ ID NO: 362 - DNA, 20 bp, Artificial sequence
  Synthetic oligonucleotide
attatgtaatttttacagac

SEQ ID NO: 363 - DNA, 20 bp, Artificial sequence
  Synthetic oligonucleotide
gcgccagctttccctcccgg

SEQ ID NO: 364 - DNA, 20 bp, Artificial sequence
  Synthetic oligonucleotide
taccacgctcctccactttt

SEQ ID NO: 365 - DNA, 20 bp, Artificial sequence
  Synthetic oligonucleotide
cccgacctccgtctctgcac

SEQ ID NO: 366 - DNA, 20 bp, Artificial sequence
  Synthetic oligonucleotide
accaaaaaataattcattct

SEQ ID NO: 367 - DNA, 20 bp, Artificial sequence
  Synthetic oligonucleotide
acctgtccattttctctcca

SEQ ID NO: 368 - DNA, 20 bp, Artificial sequence
  Synthetic oligonucleotide
actgctcctttttccaaatt

SEQ ID NO: 369 - DNA, 20 bp, Artificial sequence
  Synthetic oligonucleotide
gctggattctatccaggtcc

SEQ ID NO: 370 - DNA, 20 bp, Artificial sequence
  Synthetic oligonucleotide
ataaatatttacacccttgt

SEQ ID NO: 371 - DNA, 20 bp, Artificial sequence
  Synthetic oligonucleotide
gcgccgccgcccctcgggtc

SEQ ID NO: 372 - DNA, 20 bp, Artificial sequence
  Synthetic oligonucleotide
ccatcacttctgacctgtcc

SEQ ID NO: 373 - DNA, 20 bp, Artificial sequence
  Synthetic oligonucleotide
cttggtcttctcttaaggcc

SEQ ID NO: 374 - DNA, 20 bp, Artificial sequence
  Synthetic oligonucleotide
actgatcctaggctcaggac

SEQ ID NO: 375 - DNA, 20 bp, Artificial sequence
  Synthetic oligonucleotide
ctgtgacttaattacaagga

SEQ ID NO: 376 - DNA, 20 bp, Artificial sequence
  Synthetic oligonucleotide
tacttaaaactatttctttt

SEQ ID NO: 377 - DNA, 20 bp, Artificial sequence
  Synthetic oligonucleotide
tctatcagtcacactccaca

SEQ ID NO: 378 - DNA, 20 bp, Artificial sequence
  Synthetic oligonucleotide
gtgcattatacattctcaca

SEQ ID NO: 379 - DNA, 20 bp, Artificial sequence
  Synthetic oligonucleotide
ctataaaaagttacttaaaa

SEQ ID NO: 380 - DNA, 20 bp, Artificial sequence
  Synthetic oligonucleotide
cccgacagctcctgacaacc

SEQ ID NO: 381 - DNA, 20 bp, Artificial sequence
  Synthetic oligonucleotide
tctgctctcccctccagctc

SEQ ID NO: 382 - DNA, 20 bp, Artificial sequence
  Synthetic oligonucleotide
cggagctgcacttcccacca

SEQ ID NO: 383 - DNA, 20 bp, Artificial sequence
  Synthetic oligonucleotide
gcgcctcttctgacaccaca

SEQ ID NO: 384 - DNA, 20 bp, Artificial sequence
  Synthetic oligonucleotide
tttttctcttgccccaaaca

SEQ ID NO: 385 - DNA, 20 bp, Artificial sequence
  Synthetic oligonucleotide
cccaggaccttcttcttctc

SEQ ID NO: 386 - DNA, 20 bp, Artificial sequence
  Synthetic oligonucleotide
caggccccctgcctctctct

SEQ ID NO: 387 - DNA, 20 bp, Artificial sequence
  Synthetic oligonucleotide
aagtacctctccctcctccc

SEQ ID NO: 388 - DNA, 20 bp, Artificial sequence
  Synthetic oligonucleotide
gcctggaccgcctcctccac

SEQ ID NO: 389 - DNA, 20 bp, Artificial sequence
  Synthetic oligonucleotide
gcctccacagccctgtccta

SEQ ID NO: 390 - DNA, 20 bp, Artificial sequence
  Synthetic oligonucleotide
tccaggttctgacccccttt

SEQ ID NO: 391 - DNA, 20 bp, Artificial sequence
  Synthetic oligonucleotide
attacactcataccattgta

SEQ ID NO: 392 - DNA, 20 bp, Artificial sequence
  Synthetic oligonucleotide
aaactgatttctctttaaaa

SEQ ID NO: 393 - DNA, 20 bp, Artificial sequence
  Synthetic oligonucleotide
acctgcatttccaaatactt

SEQ ID NO: 394 - DNA, 20 bp, Artificial sequence
  Synthetic oligonucleotide
tcttttcctatcaaaaccaa

SEQ ID NO: 395 - DNA, 20 bp, Artificial sequence
  Synthetic oligonucleotide
tttctgataacatttcacaa

SEQ ID NO: 396 - DNA, 20 bp, Artificial sequence
  Synthetic oligonucleotide
ttccaagacaaagacacccc

SEQ ID NO: 397 - DNA, 20 bp, Artificial sequence
  Synthetic oligonucleotide
agcagagcccttgcccgccc

SEQ ID NO: 398 - DNA, 20 bp, Artificial sequence
  Synthetic oligonucleotide
cttttgcatataaatgaaaa

SEQ ID NO: 399 - DNA, 20 bp, Artificial sequence
  Synthetic oligonucleotide
acttgcagagatcccgccac

SEQ ID NO: 400 - DNA, 20 bp, Artificial sequence
  Synthetic oligonucleotide
aactagatttaattttttta

SEQ ID NO: 401 - DNA, 20 bp, Artificial sequence
  Synthetic oligonucleotide
acagaccctcgagtcctctc

SEQ ID NO: 402 - DNA, 20 bp, Artificial sequence
  Synthetic oligonucleotide
gttctccttcttattgaaca

SEQ ID NO: 403 - DNA, 20 bp, Artificial sequence
  Synthetic oligonucleotide
gcggtcacaatactgagcct

SEQ ID NO: 404 - DNA, 20 bp, Artificial sequence
  Synthetic oligonucleotide
ctgaccccctttcctcacat

SEQ ID NO: 405 - DNA, 20 bp, Artificial sequence
  Synthetic oligonucleotide
tggcgatccgacctgccgcc

SEQ ID NO: 406 - DNA, 20 bp, Artificial sequence
  Synthetic oligonucleotide
tcctagctaattttaaccaa

SEQ ID NO: 407 - DNA, 20 bp, Artificial sequence
  Synthetic oligonucleotide
ttgttacacgcttttacgga

SEQ ID NO: 408 - DNA, 20 bp, Artificial sequence
  Synthetic oligonucleotide
accattgtatcatcttgcta

SEQ ID NO: 409 - DNA, 20 bp, Artificial sequence
  Synthetic oligonucleotide
gtggctcttttcctatcaaa

SEQ ID NO: 410 - DNA, 20 bp, Artificial sequence
  Synthetic oligonucleotide
gcatcaaaatcatctcaatt

SEQ ID NO: 411 - DNA, 20 bp, Artificial sequence
  Synthetic oligonucleotide
aatacagaatttcctgcccc

SEQ ID NO: 412 - DNA, 20 bp, Artificial sequence
  Synthetic oligonucleotide
accaaaacactgctcctttt

SEQ ID NO: 413 - DNA, 20 bp, Artificial sequence
  Synthetic oligonucleotide
ccttcttattgaacaggatc

SEQ ID NO: 414 - DNA, 20 bp, Artificial sequence
  Synthetic oligonucleotide
cacattcatttttctcttgc

SEQ ID NO: 415 - DNA, 20 bp, Artificial sequence
  Synthetic oligonucleotide
gtttagattacactcatacc

SEQ ID NO: 416 - DNA, 20 bp, Artificial sequence
  Synthetic oligonucleotide
acctttgaacttcttgctgt

SEQ ID NO: 417 - DNA, 20 bp, Artificial sequence
  Synthetic oligonucleotide
gcgctgcctccctcactctc

SEQ ID NO: 418 - DNA, 20 bp, Artificial sequence
  Synthetic oligonucleotide
acggcacgattccagcctct

SEQ ID NO: 419 - DNA, 20 bp, Artificial sequence
  Synthetic oligonucleotide
ttacactcataccattgtat

SEQ ID NO: 420 - DNA, 20 bp, Artificial sequence
  Synthetic oligonucleotide
gggagatgacttccccctcc

SEQ ID NO: 421 - DNA, 20 bp, Artificial sequence
  Synthetic oligonucleotide
aatcattaaccaactacccc

SEQ ID NO: 422 - DNA, 20 bp, Artificial sequence
  Synthetic oligonucleotide
gcctcagttactgatcaacc

SEQ ID NO: 423 - DNA, 20 bp, Artificial sequence
  Synthetic oligonucleotide
gctgagattataccaggtgc

SEQ ID NO: 424 - DNA, 20 bp, Artificial sequence
  Synthetic oligonucleotide
gccccctgcctctctctggt

SEQ ID NO: 425 - DNA, 20 bp, Artificial sequence
  Synthetic oligonucleotide
gccagagcctcccttcccca

SEQ ID NO: 426 - DNA, 20 bp, Artificial sequence
  Synthetic oligonucleotide
tcacaccctgtcccccctca

SEQ ID NO: 427 - DNA, 20 bp, Artificial sequence
  Synthetic oligonucleotide
cccgctgccacagtcaccaa

SEQ ID NO: 428 - DNA, 20 bp, Artificial sequence
  Synthetic oligonucleotide
tccaaagtgtccaacgaccc

SEQ ID NO: 429 - DNA, 20 bp, Artificial sequence
  Synthetic oligonucleotide
tccacttttttttttccaga

SEQ ID NO: 430 - DNA, 20 bp, Artificial sequence
  Synthetic oligonucleotide
gcgatccgacctgccgccgc

SEQ ID NO: 431 - DNA, 20 bp, Artificial sequence
  Synthetic oligonucleotide
ccgccccttccctgaagctc

SEQ ID NO: 432 - DNA, 20 bp, Artificial sequence
  Synthetic oligonucleotide
tcacaatactgagcctggaa

SEQ ID NO: 433 - DNA, 20 bp, Artificial sequence
  Synthetic oligonucleotide
gttgccgtagtccttggtca

SEQ ID NO: 434 - DNA, 20 bp, Artificial sequence
  Synthetic oligonucleotide
ctgctcctttttccaaattc

SEQ ID NO: 435 - DNA, 20 bp, Artificial sequence
  Synthetic oligonucleotide
ccgacagctcctgacaaccc

SEQ ID NO: 436 - DNA, 20 bp, Artificial sequence
  Synthetic oligonucleotide
ctatcagtcacactccacac

SEQ ID NO: 437 - DNA, 20 bp, Artificial sequence
  Synthetic oligonucleotide
aactgcaaagtatataatac

SEQ ID NO: 438 - DNA, 20 bp, Artificial sequence
  Synthetic oligonucleotide
aatgaaaaaagattagaata

SEQ ID NO: 439 - DNA, 20 bp, Artificial sequence
  Synthetic oligonucleotide
cccaactccagattccagac

SEQ ID NO: 440 - DNA, 20 bp, Artificial sequence
  Synthetic oligonucleotide
tccagaaacaatctattcac

SEQ ID NO: 441 - DNA, 20 bp, Artificial sequence
  Synthetic oligonucleotide
cttaaaactatttcttttgc

SEQ ID NO: 442 - DNA, 20 bp, Artificial sequence
  Synthetic oligonucleotide
tttctcttgccccaaacacc

SEQ ID NO: 443 - DNA, 20 bp, Artificial sequence
  Synthetic oligonucleotide
ctttccccagactagttttc

SEQ ID NO: 444 - DNA, 20 bp, Artificial sequence
  Synthetic oligonucleotide
ctgctctcccctccagctca

SEQ ID NO: 445 - DNA, 20 bp, Artificial sequence
  Synthetic oligonucleotide
ctggccaacctgcctctaca

SEQ ID NO: 446 - DNA, 20 bp, Artificial sequence
  Synthetic oligonucleotide
cctcgagtcctctcgctttc

SEQ ID NO: 447 - DNA, 20 bp, Artificial sequence
  Synthetic oligonucleotide
ttggcagcctcctccgtgtt

SEQ ID NO: 448 - DNA, 20 bp, Artificial sequence
  Synthetic oligonucleotide
cgccagctttccctcccggc

SEQ ID NO: 449 - DNA, 20 bp, Artificial sequence
  Synthetic oligonucleotide
tgggcctcttgtttcaccca

SEQ ID NO: 450 - DNA, 20 bp, Artificial sequence
  Synthetic oligonucleotide
ctcgggtctcacaacaggta

SEQ ID NO: 451 - DNA, 20 bp, Artificial sequence
  Synthetic oligonucleotide
ccgacctccgtctctgcaca

SEQ ID NO: 452 - DNA, 20 bp, Artificial sequence
  Synthetic oligonucleotide
ttatgtaatttttacagaca

SEQ ID NO: 453 - DNA, 20 bp, Artificial sequence
  Synthetic oligonucleotide
cctgtccattttctctccag

SEQ ID NO: 454 - DNA, 20 bp, Artificial sequence
  Synthetic oligonucleotide
ctggattctatccaggtccc

SEQ ID NO: 455 - DNA, 20 bp, Artificial sequence
  Synthetic oligonucleotide
gtggatatcacaccctgtcc

SEQ ID NO: 456 - DNA, 20 bp, Artificial sequence
  Synthetic oligonucleotide
caggaccttcttcttctcca

SEQ ID NO: 457 - DNA, 20 bp, Artificial sequence
  Synthetic oligonucleotide
agtacctctccctcctccca

SEQ ID NO: 458 - DNA, 20 bp, Artificial sequence
  Synthetic oligonucleotide
actggttatttctatgtaat

SEQ ID NO: 459 - DNA, 20 bp, Artificial sequence
  Synthetic oligonucleotide
aggttctgaccccctttcct

SEQ ID NO: 460 - DNA, 20 bp, Artificial sequence
  Synthetic oligonucleotide
aactgatttctctttaaaaa

SEQ ID NO: 461 - DNA, 20 bp, Artificial sequence
  Synthetic oligonucleotide
accccctttcctcacatgct

SEQ ID NO: 462 - DNA, 20 bp, Artificial sequence
  Synthetic oligonucleotide
acgcccccattgctggaaaa

SEQ ID NO: 463 - DNA, 20 bp, Artificial sequence
  Synthetic oligonucleotide
ctgataacatttcacaagta

SEQ ID NO: 464 - DNA, 20 bp, Artificial sequence
  Synthetic oligonucleotide
acaggtaaactgatttctct

SEQ ID NO: 465 - DNA, 20 bp, Artificial sequence
  Synthetic oligonucleotide
ctgcatttccaaatacttgc

SEQ ID NO: 466 - DNA, 20 bp, Artificial sequence
  Synthetic oligonucleotide
gcagacatcccccccgctgc

SEQ ID NO: 467 - DNA, 20 bp, Artificial sequence
  Synthetic oligonucleotide
ccacggagcccatctggatc

SEQ ID NO: 468 - DNA, 20 bp, Artificial sequence
  Synthetic oligonucleotide
gatgatcttcaacactgtgc

SEQ ID NO: 469 - DNA, 20 bp, Artificial sequence
  Synthetic oligonucleotide
ctagctaattttaaccaatt

SEQ ID NO: 470 - DNA, 20 bp, Artificial sequence
  Synthetic oligonucleotide
actcaagccatcttggaaaa

SEQ ID NO: 471 - DNA, 20 bp, Artificial sequence
  Synthetic oligonucleotide
tgtgacttaattacaaggaa

SEQ ID NO: 472 - DNA, 20 bp, Artificial sequence
  Synthetic oligonucleotide
accacgctcctccacttttt

SEQ ID NO: 473 - DNA, 20 bp, Artificial sequence
  Synthetic oligonucleotide
catcacttctgacctgtcca

SEQ ID NO: 474 - DNA, 20 bp, Artificial sequence
  Synthetic oligonucleotide
aatgtgtgcattatacattc

SEQ ID NO: 475 - DNA, 20 bp, Artificial sequence
  Synthetic oligonucleotide
aaagttacttaaaactattt

SEQ ID NO: 476 - DNA, 20 bp, Artificial sequence
  Synthetic oligonucleotide
accagatgccatcaaagata

SEQ ID NO: 477 - DNA, 20 bp, Artificial sequence
  Synthetic oligonucleotide
aggtgtctttaaccccacac

SEQ ID NO: 478 - DNA, 20 bp, Artificial sequence
  Synthetic oligonucleotide
tcttaaggccgcctcccctc

SEQ ID NO: 479 - DNA, 20 bp, Artificial sequence
  Synthetic oligonucleotide
aaatatttacacccttgtta

SEQ ID NO: 480 - DNA, 20 bp, Artificial sequence
  Synthetic oligonucleotide
tgtgacccccccaccccttg

SEQ ID NO: 481 - DNA, 20 bp, Artificial sequence
  Synthetic oligonucleotide
actccacacacattccagtt

SEQ ID NO: 482 - DNA, 20 bp, Artificial sequence
  Synthetic oligonucleotide
accacagactgcacggacca

SEQ ID NO: 483 - DNA, 20 bp, Artificial sequence
  Synthetic oligonucleotide
gcaaaatatacatttgtgtc

(19) United States
(12) Patent Application Publication
(10) Pub. No.: US 2024/0279654 A1
(43) Pub. Date: Aug. 22, 2024

(54) COMPOUNDS FOR REDUCING PTBP1 EXPRESSION

(71) Applicant: Ionis Pharmaceuticals, Inc., Carlsbad, CA (US)

(72) Inventor: Ruben E. Valas, Oceanside, CA (US)

(73) Assignee: Ionis Pharmaceuticals, Inc., Carlsbad, CA (US)

(21) Appl. No.: 18/561,434

(22) PCT Filed: May 20, 2022
(86) PCT No.: PCT/US2022/030262

§ 371 Date: Nov. 16, 2023

Related U.S. Application Data
(60) Provisional application No. 63/191,900, filed on May 21, 2021.

(51) Int. Cl. C12N 15/113

(52) U.S. Cl. CPC C12N 15/113; C12N 2310/111; C12N 2310/315; C12N 2310/321; C12N 2310/3231; C12N 2310/3341; C12N 2310/341; C12N 2310/51

(57) ABSTRACT

Provided are oligomeric agents, oligomeric compounds, antisense agents, and pharmaceutical compositions for reducing the amount or activity of PTBP 1 RNA in a cell or animal, and in certain instances reducing the amount of PTBP 1 protein in a cell or animal. Such oligomeric agents, oligomeric compounds, antisense agents, and pharmaceutical compositions are useful to treat neurodegenerative diseases or disorders such as, but not limited to, Parkinson's disease, Huntington's disease, or Alzheimer's disease.

### SEQUENCE LISTING

The present application is being filed along with a Sequence Listing in electronic format. The Sequence Listing is provided as a file entitled BIOL0425WOSEQ_ST25.txt, created on May 10, 2022, which is 124 KB in size. The information in the electronic format of the sequence listing is incorporated herein by reference in its entirety.

### FIELD

Provided are oligomeric agents, oligomeric compounds, antisense agents, and pharmaceutical compositions for reducing the amount or activity of PTBP1 RNA in a cell or animal, and in certain instances reducing the amount of PTBP1 protein in a cell or animal. Such oligomeric agents, oligomeric compounds, antisense agents, and pharmaceutical compositions are useful to treat neurodegenerative diseases or disorders. In some embodiments, the neurodegenerative diseases or disorders include Parkinson's disease, Huntington's disease, and Alzheimer's disease.

### BACKGROUND

Polypyrimidine Tract Binding Protein 1 (PTBP1) is an RNA binding protein involved in repression of neural cell differentiation (Grammatikakis, et al., 2016, Stem Cell Invest., 3, 10). PTBP1 is expressed in most cell types. Other members of the PTB family include PTBP2 (exclusively found in the nervous system), and PTBP3 (present mainly in immune cells). The regulation of PTBP1 expression is important for the development of the nervous system. PTBP1 is expressed in embryonic stem cells and neuronal progenitor cells, and is then down-regulated during neuronal development. Down-regulation of PTBP1 induces PTBP2, which is required for neuronal maturation. In mature neurons, the expression of both PTBP1 and PTBP2 is reduced (Hu, et al., 2018, Biophys. Rep. 4, 204-214; Makeyev, et al., 2007, Mol. Cell 27, 435-448).

Reduction of PTBP1 expression in glial cells induces differentiation into neurons (Zhou, et al., 2020, Cell 181, 590-603). For example, reduction of PTBP1 in oligodendrocytes in vivo induced differentiation into striatal neurons (Weinberg, et al., 2017, The American Society of Gene and Cell Therapy, 25, 928-934). When PTBP1 expression is reduced in astrocytes in vitro, the cells differentiate to cells having a neuronal morphology, staining positive for pan-neuronal markers, and expressing neuronal genes (Qian, et al. 2020, Nature, 582, 550-556). In a chemically-induced mouse model of Parkinson's disease, reducing mouse PTBP1 expression by injection of a PTBP1-targeting shRNA-expressing virus resulted in the differentiation of astrocytes into neurons within the substantia nigra. These induced neurons restored dopamine levels in the mouse brain up to 66% of the normal level, restored the nigral-striatal circuit, and rescued motor function (Qian, 2020). Antisense oligonucleotide-mediated reduction of PTBP1 expression in vitro in mouse astrocytes induced neuronal markers, and converted dopaminergic markers. Injection of the ASO into the midbrain of transgenic mice carrying labeled astrocytes resulted in the differentiation of astrocytes to neurons displaying functional neuro-physiological properties; the PTBP1-ASO also rescued motor function in a chemically-induced mouse model of Parkinson's disease (Qian, 2020). CRISPR-mediated knockdown of PTBP1 expression in a chemically-induced mouse model of Parkinson's disease locally induced neurons expressing dopaminergic markers and alleviated motor dysfunctions (Zhou, 2020). Reduction of PTBP1 expression may also alleviate retinal injury and improve vision. In a chemically-induced mouse model of retinal injury, the CRISPR-mediated PTBP1 knockdown converted Muller glia into retinal ganglion cells (Zhou, 2020).

Currently there is a lack of acceptable options to enhance neurogenesis in subjects who have neurodegenerative diseases associated with the loss of neurons. These lost neurons may be replaced with new neurons produced by reducing the expression of PTBP1 in glial cells or other non-neural cells. It is therefore an objective herein to provide compounds and pharmaceutical compositions for the treatment of such neurodegenerative diseases or disorders.

### SUMMARY

Oligomeric agents, oligomeric compounds, antisense agents, and pharmaceutical compositions of certain embodiments described herein are useful for reducing or inhibiting PTBP1 expression in a cell or animal. In certain embodiments, PTBP1 RNA or protein levels can be reduced in a cell or animal. In certain embodiments, the subject has a neurodegenerative disease or disorder. In certain embodiments, the subject has Parkinson's disease, Huntington's disease, or Alzheimer's disease.

### DETAILED DESCRIPTION

It is to be understood that both the foregoing general description and the following detailed description are exemplary and explanatory only and are not restrictive. Herein, the use of the singular includes the plural unless specifically stated otherwise. As used herein, the use of “or” means “and/or” unless stated otherwise. Furthermore, the use of the term “including” as well as other forms, such as “includes” and “included”, is not limiting. Also, terms such as “element” or “component” encompass both elements and components comprising one unit and elements and components that comprise more than one subunit, unless specifically stated otherwise.

The section headings used herein are for organizational purposes only and are not to be construed as limiting the subject matter described. All documents, or portions of documents, cited in this application, including, but not limited to, patents, patent applications, articles, books, and treatises, are hereby expressly incorporated-by-reference for the portions of the document discussed herein, as well as in their entirety.

### Definitions

Unless specific definitions are provided, the nomenclature used in connection with, and the procedures and techniques of, analytical chemistry, synthetic organic chemistry, and medicinal and pharmaceutical chemistry described herein are those well-known and commonly used in the art. Where permitted, all patents, applications, published applications and other publications and other data referred to throughout in the disclosure are incorporated by reference herein in their entirety.

Unless otherwise indicated, the following terms have the following meanings:

As used herein, “2′-deoxynucleoside” means a nucleoside comprising a 2′-H(H) deoxyribosyl sugar moiety. In certain embodiments, a 2′-deoxynucleoside is a 2′-β-D-deoxynucleoside and comprises a 2′-β-D-deoxyribosyl sugar moiety, which has the β-D ribosyl configuration as found in naturally occurring deoxyribonucleic acids (DNA). In certain embodiments, a 2′-deoxynucleoside may comprise a modified nucleobase or may comprise an RNA nucleobase (uracil).

As used herein, “2′-MOE” means a 2′-OCH₂CH₂OCH₃ group in place of the 2′-OH group of a ribosyl sugar moiety. A “2′-MOE sugar moiety” means a sugar moiety with a 2′-OCH₂CH₂OCH₃ group in place of the 2′-OH group of a ribosyl sugar moiety. Unless otherwise indicated, a 2′-MOE sugar moiety is in the β-D-ribosyl configuration. “MOE” means O-methoxyethyl.

As used herein, “2′-MOE nucleoside” means a nucleoside comprising a 2′-MOE sugar moiety.

As used herein, “2′-OMe” means a 2′-OCH₃ group in place of the 2′-OH group of a ribosyl sugar moiety. A “2′-O-methyl sugar moiety” or “2′-OMe sugar moiety” means a sugar moiety with a 2′-OCH₃ group in place of the 2′-OH group of a ribosyl sugar moiety. Unless otherwise indicated, a 2′-OMe sugar moiety is in the β-D-ribosyl configuration.

As used herein, “2′-OMe nucleoside” means a nucleoside comprising a 2′-OMe sugar moiety.

As used herein, “2′-F” means a 2′-fluoro group in place of the 2′-OH group of a ribosyl sugar moiety. A “2′-F sugar moiety” or “2′-fluororibosyl sugar moiety” means a sugar moiety with a 2′—F group in place of the 2′-OH group of a ribosyl sugar moiety. Unless otherwise indicated, a 2′-F has the β-D ribosyl stereochemical configuration.

As used herein, “2′-F nucleoside” means a nucleoside comprising a 2′-F sugar moiety.

As used herein, “2′-substituted nucleoside” means a nucleoside comprising a 2′-substituted furanosyl sugar moiety. As used herein, “2′-substituted” in reference to a sugar moiety means a sugar moiety comprising at least one 2′-substituent group other than H or OH.

As used herein, “3′ target site” refers to the 3′-most nucleotide of a target nucleic acid which is complementary to an antisense oligonucleotide, when the antisense oligonucleotide is hybridized to the target nucleic acid.

As used herein, “5′ target site” refers to the 5′-most nucleotide of a target nucleic acid which is complementary to an antisense oligonucleotide, when the antisense oligonucleotide is hybridized to the target nucleic acid.

As used herein, “5-methylcytosine” means a cytosine modified with a methyl group attached to the 5 position. A 5-methylcytosine is a modified nucleobase.

As used herein, “abasic sugar moiety” means a sugar moiety of a nucleoside that is not attached to a nucleobase. Such abasic sugar moieties are sometimes referred to in the art as “abasic nucleosides.”

As used herein, “administration” or “administering” means providing a pharmaceutical agent or composition to an animal.

As used herein, “ameliorate” in reference to a treatment means improvement in at least one symptom or hallmark relative to the same symptom or hallmark in the absence of the treatment. In certain embodiments, amelioration is the reduction in the severity or frequency of a symptom or hallmark or the delayed onset or slowing of progression in the severity or frequency of a symptom or hallmark. The progression or severity of indicators may be determined by subjective or objective measures, which are known to those skilled in the art.

As used herein, “animal” means a human or non-human animal.

As used herein, “cerebrospinal fluid” or “CSF” means the fluid filling the space around the brain and spinal cord. “Artificial cerebrospinal fluid” or “aCSF” means a prepared or manufactured fluid that has certain properties (e.g., osmolarity, pH, and/or electrolytes) similar to cerebrospinal fluid and is biocompatible with CSF.

As used herein, “bicyclic nucleoside” or “BNA” means a nucleoside comprising a bicyclic sugar moiety.

As used herein, “bicyclic sugar” or “bicyclic sugar moiety” means a modified sugar moiety comprising two rings, wherein the second ring is formed via a bridge connecting two of the atoms in the first ring thereby forming a bicyclic structure. In certain embodiments, the first ring of the bicyclic sugar moiety is a furanosyl moiety. In certain embodiments, the furanosyl sugar moiety is a ribosyl sugar moiety. In certain embodiments, the bicyclic sugar moiety does not comprise a furanosyl moiety.

As used herein, “chirally enriched population” means a plurality of molecules of identical molecular formula, wherein the number or percentage of molecules within the population that contain a particular stereochemical configuration at a particular chiral center is greater than the number or percentage of molecules expected to contain the same particular stereochemical configuration at the same particular chiral center within the population if the particular chiral center were stereorandom. Chirally enriched populations of molecules having multiple chiral centers within each molecule may contain one or more stereorandom chiral centers. In certain embodiments, the molecules are modified oligonucleotides. In certain embodiments, the molecules are oligomeric compounds comprising modified oligonucleotides.

As used herein, “cleavable moiety” means a bond or group of atoms that is cleaved under physiological conditions, for example, inside a cell, an animal, or a human.

As used herein, “complementary” in reference to an oligonucleotide means that at least 70% of the nucleobases of the oligonucleotide and the nucleobases of another nucleic acid or one or more regions thereof are capable of hydrogen bonding with one another when the nucleobase sequence of the oligonucleotide and the other nucleic acid are aligned in opposing directions. “Complementary region” in reference to a region of an oligonucleotide means that at least 70% of the nucleobases of that region and the nucleobases of another nucleic acid or one or more regions thereof are capable of hydrogen bonding with one another when the nucleobase sequence of the oligonucleotide and the other nucleic acid are aligned in opposing directions. Complementary nucleobases mean nucleobases that are capable of forming hydrogen bonds with one another. Complementary nucleobase pairs include adenine (A) and thymine (T), adenine (A) and uracil (U), cytosine (C) and guanine (G), 5-methylcytosine (mC) and guanine (G). Certain modified nucleobases that pair with natural nucleobases or with other modified nucleobases are known in the art and are not considered complementary nucleobases as defined herein unless indicated otherwise. For example, inosine can pair, but is not considered complementary, with adenosine, cytosine, or uracil. Complementary oligonucleotides and/or nucleic acids need not have nucleobase complementarity at each nucleoside. Rather, some mismatches are tolerated. As used herein, “fully complementary” or “100% complementary” in reference to oligonucleotides means that oligonucleotides are complementary to another oligonucleotide or nucleic acid at each nucleoside of the oligonucleotide.

As used herein, “conjugate group” means a group of atoms that is directly attached to an oligonucleotide. Conjugate groups include a conjugate moiety and a conjugate linker that attaches the conjugate moiety to the oligonucleotide.

As used herein, “conjugate linker” means a single bond or a group of atoms comprising at least one bond that connects a conjugate moiety to an oligonucleotide.

As used herein, “conjugate moiety” means a group of atoms that modifies one or more properties of a molecule compared to the identical molecule lacking the conjugate moiety, including but not limited to pharmacodynamics, pharmacokinetics, stability, binding, absorption, tissue distribution, cellular distribution, cellular uptake, charge and clearance.

As used herein, “contiguous” in the context of an oligonucleotide refers to nucleosides, nucleobases, sugar moieties, or internucleoside linkages that are immediately adjacent to each other. For example, “contiguous nucleobases” means nucleobases that are immediately adjacent to each other in a sequence.

As used herein, “constrained ethyl” or “cEt” or “cEt modified sugar moiety” means a β-D ribosyl bicyclic sugar moiety wherein the second ring of the bicyclic sugar is formed via a bridge connecting the 4′-carbon and the 2′-carbon of the β-D ribosyl sugar moiety, wherein the bridge has the formula 4′-CH(CH₃)—O-2′, and wherein the methyl group of the bridge is in the S configuration.

As used herein, “cEt nucleoside” means a nucleoside comprising a cEt modified sugar moiety.

As used herein, “deoxy region” means a region of 5-12 contiguous nucleotides, wherein at least 70% of the nucleosides comprise a 2′-β-D-deoxyribosyl sugar moiety. In certain embodiments, a deoxy region is the gap of a gapmer.

As used herein, “diluent” means an ingredient in a composition that lacks pharmacological activity, but is pharmaceutically necessary or desirable. For example, the diluent in an injected composition can be a liquid, e.g., aCSF, PBS, or saline solution.

As used herein, “double-stranded” in reference to a region or an oligonucleotide means a duplex formed by complementary strands of nucleic acids (including, but not limited to oligonucleotides) hybridized to one another. In certain embodiments, the two strands of a double-stranded region are separate molecules. In certain embodiments, the two strands are regions of the same molecule that has folded onto itself (e.g., a hairpin structure).

As used herein, “hotspot region” is a range of nucleobases on a target nucleic acid that is amenable to oligomeric agent or oligomeric compound-mediated reduction of the amount or activity of the target nucleic acid.

As used herein, “internucleoside linkage” is the covalent linkage between adjacent nucleosides in an oligonucleotide. As used herein “modified internucleoside linkage” means any internucleoside linkage other than a phosphodiester internucleoside linkage. Phosphorothioate internucleoside linkage” or “PS internucleoside linkage” is a modified internucleoside linkage in which one of the non-bridging oxygen atoms of a phosphodiester internucleoside linkage is replaced with a sulfur atom.

As used herein, “linked nucleosides” are nucleosides that are connected in a contiguous sequence (i.e., no additional nucleosides are presented between those that are linked).

As used herein, “linker-nucleoside” means a nucleoside that links, either directly or indirectly, an oligonucleotide to a conjugate moiety. Linker-nucleosides are located within the conjugate linker of an oligomeric compound. Linker-nucleosides are not considered part of the oligonucleotide portion of an oligomeric compound even if they are contiguous with the oligonucleotide.

As used herein, “mismatch” or “non-complementary” means a nucleobase of a first nucleic acid sequence that is not complementary with the corresponding nucleobase of a second nucleic acid sequence or target nucleic acid when the first and second nucleic acid sequences are aligned in opposing directions.

As used herein, “modified nucleoside” means a nucleoside comprising a modified nucleobase and/or a modified sugar moiety.

As used herein, “motif” means the pattern of unmodified and/or modified sugar moieties, nucleobases, and/or internucleoside linkages, in an oligonucleotide.

As used herein, “non-bicyclic modified sugar moiety” means a modified sugar moiety that comprises a modification, such as a substituent, that does not form a bridge between two atoms of the sugar to form a second ring.

As used herein, “nucleobase” means an unmodified nucleobase or a modified nucleobase. A nucleobase is a heterocyclic moiety. As used herein an “unmodified nucleobase” is adenine (A), thymine (T), cytosine (C), uracil (U), or guanine (G). As used herein, a “modified nucleobase” is a group of atoms other than unmodified A, T, C, U, or G capable of pairing with at least one other nucleobase. A “5-methylcytosine” is a modified nucleobase. A universal base is a modified nucleobase that can pair with any one of the five unmodified nucleobases.

As used herein, “nucleobase sequence” means the order of contiguous nucleobases in a nucleic acid or oligonucleotide independent of any sugar or internucleoside linkage modification.

As used herein, “nucleoside” means a compound or fragment of a compound comprising a nucleobase and a sugar moiety. The nucleobase and sugar moiety are each, independently, unmodified or modified.

As used herein, “oligomeric agent” means an oligomeric compound and optionally one or more additional features, such as a second oligomeric compound. An oligomeric agent may be a single-stranded oligomeric compound or may be an oligomeric duplex formed by two complementary oligomeric compounds.

As used herein, “oligomeric compound” means an oligonucleotide and optionally one or more additional features, such as a conjugate group or terminal group. An oligomeric compound may be paired with a second oligomeric compound that is complementary to the first oligomeric compound or may be unpaired. A “singled-stranded oligomeric compound” is an unpaired oligomeric compound.

The term “oligomeric duplex” means a duplex formed by two oligomeric compounds having complementary nucleobase sequences.

As used herein, “oligonucleotide” means a strand of linked nucleosides connected via internucleoside linkages, wherein each nucleoside and internucleoside linkage may be modified or unmodified. Unless otherwise indicated, oligonucleotides consist of 8-50 linked nucleosides.

As used herein, “modified oligonucleotide” means an oligonucleotide, wherein at least one nucleoside or internucleoside linkage is modified. As used herein, “unmodified oligonucleotide” means an oligonucleotide that does not comprise any nucleoside modifications or internucleoside modifications.

As used herein, “pharmaceutically acceptable carrier or diluent” means any substance suitable for use in administering to an animal. Certain such carriers enable pharmaceutical compositions to be formulated as, for example, tablets, pills, dragees, capsules, liquids, gels, syrups, slurries, suspension and lozenges for the oral ingestion by a subject. In certain embodiments, a pharmaceutically acceptable carrier or diluent is sterile water, sterile saline, sterile buffer solution or sterile artificial cerebrospinal fluid.

As used herein “pharmaceutically acceptable salts” means physiologically and pharmaceutically acceptable salts of compounds. Pharmaceutically acceptable salts retain the desired biological activity of the parent compound and do not impart undesired toxicological effects thereto.

As used herein “pharmaceutical composition” means a mixture of substances suitable for administering to a subject. For example, a pharmaceutical composition may comprise an oligomeric compound and a sterile aqueous solution. In certain embodiments, a pharmaceutical composition shows activity in free uptake assay in certain cell lines.

As used herein “prodrug” means a therapeutic agent in a first form outside the body that is converted to a second form within an animal or cells thereof. Typically, conversion of a prodrug within the animal is facilitated by the action of an enzymes (e.g., endogenous or viral enzyme) or chemicals present in cells or tissues and/or by physiologic conditions. In certain embodiments, the first form of the prodrug is less active than the second form.

As used herein, “reducing or inhibiting the amount or activity” refers to a reduction or blockade of the transcriptional expression or activity relative to the transcriptional expression or activity in an untreated or control sample and does not necessarily indicate a total elimination of transcriptional expression or activity.

As used herein, “single-stranded” means a nucleic acid (including but not limited to an oligonucleotide) that is unpaired and is not part of a duplex. Single-stranded compounds are capable of hybridizing with complementary nucleic acids to form duplexes, at which point they are no longer single-stranded.

As used herein, “stabilized phosphate group” refers to a 5′-chemical moiety that results in stabilization of a 5′-phosphate moiety of the 5′-terminal nucleoside of an oligonucleotide, relative to the stability of an unmodified 5′-phosphate of an unmodified nucleoside under biologic conditions. Such stabilization of a 5′-phosphate group includes but is not limited to resistance to removal by phosphatases. Stabilized phosphate groups include, but are not limited to, 5′-vinyl phosphonates and 5′-cyclopropyl phosphonate.

As used herein, “standard in vitro cell assay” means the assays described in Example 1 and reasonable variations thereof.

As used herein, “stereorandom chiral center” in the context of a population of molecules of identical molecular formula means a chiral center having a random stereochemical configuration. For example, in a population of molecules comprising a stereorandom chiral center, the number of molecules having the (S) configuration of the stereorandom chiral center may be but is not necessarily the same as the number of molecules having the (R) configuration of the stereorandom chiral center. The stereochemical configuration of a chiral center is considered random when it is the result of a synthetic method that is not designed to control the stereochemical configuration. In certain embodiments, a stereorandom chiral center is a stereorandom phosphorothioate internucleoside linkage.

As used herein, “subject” means a human or non-human animal. In certain embodiments, the subject is a human.

As used herein, “sugar moiety” means an unmodified sugar moiety or a modified sugar moiety. As used herein, “unmodified sugar moiety” means a 2′-OH(H) ribosyl moiety, as found in RNA (an “unmodified RNA sugar moiety”), or a 2′-H(H) deoxyribosyl sugar moiety, as found in DNA (an “unmodified DNA sugar moiety”). Unmodified sugar moieties have one hydrogen at each of the 1′, 3′, and 4′ positions, an oxygen at the 3′ position, and two hydrogens at the 5′ position. As used herein, “modified sugar moiety” or “modified sugar” means a modified furanosyl sugar moiety or a sugar surrogate.

As used herein, “sugar surrogate” means a modified sugar moiety having other than a furanosyl moiety that can link a nucleobase to another group, such as an internucleoside linkage, conjugate group, or terminal group in an oligonucleotide. Modified nucleosides comprising sugar surrogates can be incorporated into one or more positions within an oligonucleotide and such oligonucleotides are capable of hybridizing to complementary oligomeric compounds or target nucleic acids.

As used herein, “symptom or hallmark” means any physical feature or test result that indicates the existence or extent of a disease or disorder. In certain embodiments, a symptom is apparent to a subject or to a medical professional examining or testing said subject. In certain embodiments, a hallmark is apparent upon invasive diagnostic testing, including, but not limited to, post-mortem tests.

As used herein, “target nucleic acid” and “target RNA” mean a nucleic acid that an oligomeric compound is designed to affect. Target RNA means an RNA transcript and includes pre-mRNA and mature mRNA unless otherwise specified.

As used herein, “target region” means a portion of a target nucleic acid to which an oligomeric compound is designed to hybridize.

As used herein, “terminal group” means a chemical group or group of atoms that is covalently linked to a terminus of an oligonucleotide.

As used herein, “treating” means improving a subject's disease or condition by administering an oligomeric agent, an oligomeric compound, an oligomeric duplex, or an antisense agent described herein. In certain embodiments, treating a subject improves a symptom relative to the same symptom in the absence of the treatment. In certain embodiments, treatment reduces in the severity or frequency of a symptom, or delays the onset of a symptom, slows the progression of a symptom, or slows the severity or frequency of a symptom.

As used herein, “antisense activity” means any detectable and/or measurable change attributable to the hybridization of an antisense compound to its target nucleic acid. In certain embodiments, antisense activity is a decrease in the amount or expression of a target nucleic acid or protein encoded by such target nucleic acid compared to target nucleic acid levels or target protein levels in the absence of the antisense compound. In certain embodiments, antisense activity is the modulation of splicing of a target pre-mRNA.

As used herein, “antisense agent” means an antisense compound and optionally one or more additional features, such as a sense compound.

As used herein, “antisense compound” means an antisense oligonucleotide and optionally one or more additional features, such as a conjugate group.

As used herein, “sense compound” means a sense oligonucleotide and optionally one or more additional features, such as a conjugate group.

As used herein, “antisense oligonucleotide” means an oligonucleotide, including the oligonucleotide portion of an antisense compound, that is capable of hybridizing to a target nucleic acid and is capable of at least one antisense activity. Antisense oligonucleotides include but are not limited to antisense RNAi oligonucleotides and antisense RNase H oligonucleotides.

As used herein, “sense oligonucleotide” means an oligonucleotide, including the oligonucleotide portion of a sense compound, that is capable of hybridizing to an antisense oligonucleotide.

As used herein, “gapmer” means a modified oligonucleotide comprising an internal region positioned between external regions having one or more nucleosides, wherein the nucleosides comprising the internal region are chemically distinct from the nucleoside or nucleosides comprising the external regions, and wherein the modified oligonucleotide supports RNAse H cleavage. The internal region may be referred to as the “gap” and the external regions may be referred to as the “wings.” In certain embodiments, the internal region is a deoxy region. The positions of the internal region or gap refer to the order of the nucleosides of the internal region and are counted starting from the 5′-end of the internal region. Unless otherwise indicated, “gapmer” refers to a sugar motif. In certain embodiments, each nucleoside of the gap is a 2′-β-D-deoxynucleoside. In certain embodiments, the gap comprises one 2′-substituted nucleoside at position 1, 2, 3, 4, or 5 of the gap, and the remainder of the nucleosides of the gap are 2′-β-D-deoxynucleosides. As used herein, the term “MOE gapmer” indicates a gapmer having a gap comprising 2′-β-D-deoxynucleosides and wings comprising 2′-MOE nucleosides. As used herein, the term “mixed wing gapmer” indicates a gapmer having wings comprising modified nucleosides comprising at least two different sugar modifications. Unless otherwise indicated, a gapmer may comprise one or more modified internucleoside linkages and/or modified nucleobases and such modifications do not necessarily follow the gapmer pattern of the sugar modifications.

As used herein, “cell-targeting moiety” means a conjugate group or portion of a conjugate group that is capable of binding to a particular cell type or particular cell types.

As used herein, “hybridization” means the annealing of oligonucleotides and/or nucleic acids. While not limited to a particular mechanism, the most common mechanism of hybridization involves hydrogen bonding, which may be Watson-Crick, Hoogsteen or reversed Hoogsteen hydrogen bonding, between complementary nucleobases. In certain embodiments, complementary nucleic acid molecules include, but are not limited to, an antisense compound and a nucleic acid target. In certain embodiments, complementary nucleic acid molecules include, but are not limited to, an oligonucleotide and a nucleic acid target.

As used herein, “RNA” means an RNA transcript and includes pre-mRNA and mature mRNA unless otherwise specified.

As used herein, “RNAi agent” means an antisense agent that acts, at least in part, through RISC or Ago2 to modulate a target nucleic acid and/or protein encoded by a target nucleic acid. RNAi agents include, but are not limited to double-stranded siRNA, single-stranded RNAi (ssRNAi), and microRNA, including microRNA mimics. RNAi agents may comprise conjugate groups and/or terminal groups. In certain embodiments, an RNAi agent modulates the amount and/or activity, of a target nucleic acid. The term RNAi agent excludes antisense agents that act through RNase H.

As used herein, “RNase H agent” means an antisense agent that acts through RNase H to modulate a target nucleic acid and/or protein encoded by a target nucleic acid. In certain embodiments, RNase H agents are single-stranded. In certain embodiments, RNase H agents are double-stranded. RNase H compounds may comprise conjugate groups and/or terminal groups. In certain embodiments, an RNase H agent modulates the amount and/or activity of a target nucleic acid. The term RNase H agent excludes antisense agents that act principally through RISC/Ago2.

As used herein, “antisense RNase H oligonucleotide” means an oligonucleotide comprising a region that is complementary to a target sequence, and which includes at least one chemical modification suitable for RNase H-mediated nucleic acid reduction.

As used herein, “antisense RNAi oligonucleotide” means an oligonucleotide comprising a region that is complementary to a target sequence, and which includes at least one chemical modification suitable for RNAi-mediated nucleic acid reduction.

### Certain Embodiments

The present disclosure provides the following non-limiting numbered embodiments:

Embodiment 1. An oligomeric compound comprising a modified oligonucleotide consisting of 8 to 80 linked nucleosides, wherein the nucleobase sequence of the modified oligonucleotide is at least 80% complementary to the nucleobase sequence of an equal length portion of a PTBP1 nucleic acid, and wherein the modified oligonucleotide has at least one modification selected from a modified sugar moiety and a modified internucleoside linkage.

Embodiment 2. The oligomeric compound of embodiment 1, wherein the PTBP1 nucleic acid has the nucleobase sequence of SEQ ID NO: 1 or SEQ ID NO: 2.

Embodiment 3. The oligomeric compound of embodiment 1 or embodiment 2, wherein the nucleobase sequence of the modified oligonucleotide is at least 80% complementary to the nucleobase sequence of an equal length portion within nucleobases 16,938-16,960; nucleobases 17,538-17,573; or nucleobases 17,988-18,016 of SEQ ID NO: 1.

Embodiment 4. The oligomeric compound of any of embodiments 1-3, wherein the nucleobase sequence of the modified oligonucleotide is at least 85%, at least 90%, at least 95%, or 100% complementary to the nucleobase sequence of an equal length portion of the PTBP1 nucleic acid.

Embodiment 5. An oligomeric compound, wherein the oligomeric compound comprises a modified oligonucleotide consisting of 8 to 80 linked nucleosides, wherein the nucleobase sequence of the modified oligonucleotide comprises at least 8, at least 9, at least 10, at least 11, at least 12, at least 13, at least 14, at least 15, at least 16, at least 17, at least 18, at least 19, or 20 contiguous nucleobases of any of the nucleobase sequences of SEQ ID NOs: 21-483, and wherein the modified oligonucleotide comprises at least one modification selected from a modified sugar moiety and a modified internucleoside linkage.

Embodiment 6. The oligomeric compound of embodiment 5, wherein the nucleobase sequence of the modified oligonucleotide comprises the nucleobase sequence of any of SEQ ID NOs: 21-483.

Embodiment 7. The oligomeric compound of embodiment 6, wherein the modified oligonucleotide has a nucleobase sequence consisting of the nucleobase sequence of any of SEQ ID NOs: 21-483.

Embodiment 8. The oligomeric compound of any of embodiments 5-7, wherein the modified oligonucleotide has a nucleobase sequence comprising at least 8, at least 9, at least 10, at least 11, at least 12, at least 13, at least 14, at least 15, at least 16, at least 17, at least 18, at least 19, or 20 contiguous nucleobases of the nucleobase sequence of
SEQ ID NO: 21, 253, 330, or 407;
SEQ ID NO: 84, 145, 170, 239, 318, 339, or 408; or
SEQ ID NO: 21, 253, 330, or 407.

Embodiment 9. The oligomeric compound of embodiment 8, wherein the modified oligonucleotide consists of 10-80, 20 to 80, 10-50, 20-50, 10-30, or 20-30 linked nucleosides, wherein the nucleobase sequence of the modified oligonucleotide comprises the nucleobase sequence of
SEQ ID NO: 21, 253, 330, or 407;
SEQ ID NO: 84, 145, 170, 239, 318, 339, or 408; or
SEQ ID NO: 21, 253, 330, or 407.

Embodiment 10. The oligomeric compound of embodiment 9, wherein the modified oligonucleotide has a nucleobase sequence consisting of the nucleobase sequence of
SEQ ID NO: 21, 253, 330, or 407;
SEQ ID NO: 84, 145, 170, 239, 318, 339, or 408; or
SEQ ID NO: 21, 253, 330, or 407.

Embodiment 11. The oligomeric compound of any of embodiments 5-10, wherein the nucleobase sequence of the modified oligonucleotide is at least 85%, at least 90%, at least 95%, or 100% complementary to the nucleobase sequence of an equal length portion of a PTBP1 nucleic acid, wherein the PTBP1 nucleic acid has the nucleobase sequence of SEQ ID NO: 1 or SEQ ID NO: 2.

Embodiment 12. The oligomeric compound of any of embodiments 1-11, wherein the modified oligonucleotide consists of 10 to 25, 10 to 30, 10 to 50, 12 to 20, 12 to 25, 12 to 30, 12 to 50, 13 to 20, 13 to 25, 13 to 30, 13 to 50, 14 to 20, 14 to 25, 14 to 30, 14 to 50, 15 to 20, 15 to 25, 15 to 30, 15 to 50, 16 to 18, 16 to 20, 16 to 25, 16 to 30, 16 to 50, 17 to 20, 17 to 25, 17 to 30, 17 to 50, 18 to 20, 18 to 22, 18 to 25, 18 to 30, 18 to 50, 19 to 20, 19 to 25, 19 to 30, 19 to 50, 20 to 25, 20 to 30, 20 to 50, 21 to 25, 21 to 30, 21 to 50, 22 to 25, 22 to 30, 22 to 50, 23 to 25, 23 to 30, or 23 to 50 linked nucleosides.

Embodiment 13. The oligomeric compound of any of embodiments 1-12, wherein the modified oligonucleotide consists of 20 linked nucleosides.

Embodiment 14. The oligomeric compound of any of embodiments 1-13, wherein at least one nucleoside of the modified oligonucleotide comprises a modified sugar moiety.

Embodiment 15. The oligomeric compound of embodiment 14, wherein the modified sugar moiety comprises a bicyclic sugar moiety.

Embodiment 16. The oligomeric compound of embodiment 15, wherein the bicyclic sugar moiety comprises a 2′-4′ bridge selected from —O—CH₂—; and —O—CH(CH₃)—.

Embodiment 17. The oligomeric compound of embodiment 14, wherein the modified sugar moiety comprises a non-bicyclic modified sugar moiety.

Embodiment 18. The oligomeric compound of embodiment 17, wherein the non-bicyclic modified sugar moiety is a 2′-MOE sugar moiety or 2′-OMe sugar moiety.

Embodiment 19. The oligomeric compound of any of embodiments 1-18, wherein at least one nucleoside of the modified oligonucleotide compound comprises a sugar surrogate.

Embodiment 20. The oligomeric compound of any of embodiments 1-19, wherein the modified oligonucleotide comprises at least one modified internucleoside linkage.

Embodiment 21. The oligomeric compound of embodiment 20, wherein at least one modified internucleoside linkage is a phosphorothioate internucleoside linkage.

Embodiment 22. The oligomeric compound of embodiment 20 or embodiment 21, wherein each internucleoside linkage is a modified internucleoside linkage.

Embodiment 23. The oligomeric compound of embodiment 22, wherein each internucleoside linkage is a phosphorothioate internucleoside linkage.

Embodiment 24. The oligomeric compound of embodiment 20 or embodiment 21, wherein at least one internucleoside linkage of the modified oligonucleotide is a phosphodiester internucleoside linkage.

Embodiment 25. The oligomeric compound of any of embodiments 1-21 or 24, wherein each internucleoside linkage of the modified oligonucleotide is independently selected from a phosphodiester internucleoside linkage or a phosphorothioate internucleoside linkage.

Embodiment 26. The oligomeric compound of any of embodiments 1-21 or 24-25, wherein at least 10, at least 11, at least 12, at least 13, at least 14, at least 15, at least 16, at least 17, or at least 18 internucleoside linkages of the modified oligonucleotide are phosphorothioate internucleoside linkages.

Embodiment 27. The oligomeric compound of any of embodiments 1-21 or 24-26, wherein the internucleoside linkage motif of the modified oligonucleotide is 5′-sooosssssssssssooss-3′, wherein each “s” represents a phosphorothioate internucleoside linkage and each “o” represents a phosphodiester internucleoside linkage.

Embodiment 28. The oligomeric compound of any of embodiments 1-27, wherein the modified oligonucleotide comprises at least one modified nucleobase.

Embodiment 29. The oligomeric compound of embodiment 28, wherein the modified nucleobase is 5-methylcytosine.

Embodiment 30. The oligomeric compound of embodiment 29, wherein each cytosine is a 5-methylcytosine.

Embodiment 31. The oligomeric compound of any of embodiments 1-30, wherein the modified oligonucleotide comprises a deoxy region.

Embodiment 32. The oligomeric compound of embodiment 31, wherein each nucleoside of the deoxy region is a 2′-β-D-deoxynucleoside.

Embodiment 33. The oligomeric compound of embodiment 31 or embodiment 32, wherein the deoxy region consists of 6, 7, 8, 9, 10, or 6-10 linked nucleosides.

Embodiment 34. The oligomeric compound of any of embodiments 31-33, wherein each nucleoside immediately adjacent to the deoxy region comprises a modified sugar moiety.

Embodiment 35. The oligomeric compound of any of embodiments 31-34, wherein the deoxy region is flanked on the 5′-side by a 5′ external region consisting of 1-6 linked 5′ external region nucleosides and on the 3′-side by a 3′ external region consisting of 1-6 linked 3′ external region nucleosides; wherein
the 3′-most nucleoside of the 5′ external region comprises a modified sugar moiety; and
the 5′-most nucleoside of the 3′ external region comprises a modified sugar moiety.

Embodiment 36. The oligomeric compound of embodiment 35, wherein each nucleoside of the 3′ external region comprises a modified sugar moiety.

Embodiment 37. The oligomeric compound of embodiment 35 or embodiment 36, wherein each nucleoside of the 5′ external region comprises a modified sugar moiety.

Embodiment 38. The oligomeric compound of embodiment 37, wherein the modified oligonucleotide has:
a 5′ external region consisting of 5 linked nucleosides;
a deoxy region consisting of 10 linked nucleosides; and
a 3′ external region consisting of 5 linked nucleosides;

wherein each of the 5′ external region nucleosides and each of the 3′ external region nucleosides is a 2′-MOE nucleoside.

Embodiment 39. The oligomeric compound of embodiment 37, wherein the modified oligonucleotide has:
a 5′ external region consisting of 1-6 linked nucleosides;
a deoxy region consisting of 6-10 linked nucleosides; and
a 3′ external region consisting of 1-6 linked nucleosides;

wherein each of the 5′ external region nucleosides and each of the 3′ external region nucleosides is a cEt nucleoside or a 2′-MOE nucleoside; and each of the deoxy region nucleosides is a 2′-β-D-deoxynucleoside.

Embodiment 40. The oligomeric compound of any of embodiments 1-39, wherein the modified oligonucleotide has a sugar motif of 5′-eeeeeddddddddddeeeee-3′, wherein each “d” represents a 2′-β-D-deoxyribosyl sugar moiety, and each “e” represents a 2′-MOE sugar moiety.

Embodiment 41. The oligomeric compound of any of embodiments 1-40, consisting of the modified oligonucleotide.

Embodiment 42. The oligomeric compound of any of embodiments 1-40, wherein the oligomeric compound comprises a conjugate group.

Embodiment 43. The oligomeric compound of embodiment 42, wherein the conjugate group comprises a conjugate linker and a conjugate moiety.

Embodiment 44. The oligomeric compound of embodiment 43, wherein the conjugate linker consists of a single bond.

Embodiment 45. The oligomeric compound of any of embodiments 43-44, wherein the conjugate linker is cleavable.

Embodiment 46. The oligomeric compound of embodiment 43 or embodiment 45, wherein the conjugate linker comprises 1-3 linker-nucleosides.

Embodiment 47. The oligomeric compound of any of embodiments 43-45, wherein the conjugate linker does not comprise any linker nucleosides.

Embodiment 48. The oligomeric compound of any of embodiments 42-47, wherein the conjugate group is attached to the modified oligonucleotide at the 5′-end of the modified oligonucleotide.

Embodiment 49. The oligomeric compound of any of embodiments 42-47, wherein the conjugate group is attached to the modified oligonucleotide at the 3′-end of the modified oligonucleotide.

Embodiment 50. The oligomeric compound of any of embodiments 1 to 49, wherein the oligomeric compound comprises a terminal group.

Embodiment 51. The oligomeric compound of embodiment 50, wherein the terminal group is an abasic sugar moiety.

Embodiment 52. The oligomeric compound of any of embodiments 1-51, wherein the oligomeric compound is a singled-stranded oligomeric compound.

Embodiment 53. A chirally enriched population of oligomeric compounds of any of embodiments 1-52, wherein the population is enriched for modified oligonucleotides comprising at least one particular phosphorothioate internucleoside linkage having a particular stereochemical configuration.

Embodiment 54. The chirally enriched population of embodiment 53, wherein the population is enriched for modified oligonucleotides comprising at least one particular phosphorothioate internucleoside linkage having the (Sp) or (Rp) configuration.

Embodiment 55. The chirally enriched population of embodiment 53, wherein the population is enriched for modified oligonucleotides having a particular, independently selected stereochemical configuration at each phosphorothioate internucleoside linkage.

Embodiment 56. The chirally enriched population of embodiment 53, wherein the population is enriched for modified oligonucleotides having the (Rp) configuration at one particular phosphorothioate internucleoside linkage and the (Sp) configuration at each of the remaining phosphorothioate internucleoside linkages.

Embodiment 57. The chirally enriched population of embodiment 53, wherein the population is enriched for modified oligonucleotides having at least 3 contiguous phosphorothioate internucleoside linkages in the Sp, Sp, and Rp configurations, in the 5′ to 3′ direction.

Embodiment 58. A population of oligomeric compounds of any of embodiments 1-52, wherein all of the phosphorothioate internucleoside linkages of the modified oligonucleotide are stereorandom.

Embodiment 59. An oligomeric duplex, comprising a first oligomeric compound and a second oligomeric compound comprising a second modified oligonucleotide, wherein the first oligomeric compound is an oligomeric compound of any of embodiments 1-52.

Embodiment 60. The oligomeric duplex of embodiment 59, wherein the second modified oligonucleotide consists of 8 to 80 linked nucleosides, and wherein the nucleobase sequence of the second modified oligonucleotide comprises a complementary region of at least 8 nucleobases that is at least 90% complementary to the nucleobase sequence of an equal length portion of the first modified oligonucleotide.

Embodiment 61. The oligomeric duplex of embodiment 59 or embodiment 60, wherein the modified oligonucleotide of the first oligomeric compound comprises a 5′-stabilized phosphate group.

Embodiment 62. The oligomeric duplex of embodiment 61, wherein the stabilized phosphate group comprises a cyclopropyl phosphonate or a vinyl phosphonate.

Embodiment 63. The oligomeric duplex of any of embodiments 59-62, wherein the modified oligonucleotide of the first oligomeric compound comprises a glycol nucleic acid (GNA) sugar surrogate.

Embodiment 64. The oligomeric duplex of any of embodiments 59-63, wherein the modified oligonucleotide of the first oligomeric compound comprises a 2′-NMA sugar moiety.

Embodiment 65. The oligomeric duplex of any of embodiments 59-64, wherein at least one nucleoside of the second modified oligonucleotide comprises a modified sugar moiety.

Embodiment 66. The oligomeric duplex of embodiment 65, wherein the modified sugar moiety of the second modified oligonucleotide comprises a bicyclic sugar moiety.

Embodiment 67. The oligomeric duplex of embodiment 66, wherein the bicyclic sugar moiety of the second modified oligonucleotide comprises a 2′-4′ bridge selected from —O—CH₂—; and —O—CH(CH₃)—.

Embodiment 68. The oligomeric duplex of embodiment 66, wherein the modified sugar moiety of the second modified oligonucleotide comprises a non-bicyclic modified sugar moiety.

Embodiment 69. The oligomeric duplex of embodiment 68, wherein the non-bicyclic modified sugar moiety of the second modified oligonucleotide is a 2′-MOE sugar moiety, a 2′-F sugar moiety, or 2′-OMe sugar moiety.

Embodiment 70. The oligomeric duplex of any of embodiments 59-65, wherein at least one nucleoside of the second modified oligonucleotide comprises a sugar surrogate.

Embodiment 71. The oligomeric duplex of any of embodiments 59-70, wherein the second modified oligonucleotide comprises at least one modified internucleoside linkage.

Embodiment 72. The oligomeric duplex of embodiment 71, wherein at least one modified internucleoside linkage of the second modified oligonucleotide is a phosphorothioate internucleoside linkage.

Embodiment 73. The oligomeric duplex of any of embodiments 59-72, wherein at least one internucleoside linkage of the second modified oligonucleotide is a phosphodiester internucleoside linkage.

Embodiment 74. The oligomeric duplex of any of embodiments 59-73, wherein each internucleoside linkage of the second modified oligonucleotide is independently selected from a phosphodiester or a phosphorothioate internucleoside linkage.

Embodiment 75. The oligomeric duplex of any of embodiments 59-74, wherein the second modified oligonucleotide comprises at least one modified nucleobase.

Embodiment 76. The oligomeric duplex of embodiment 75, wherein the modified nucleobase of the second modified oligonucleotide is 5-methylcytosine.

Embodiment 77. The oligomeric duplex of any of embodiments 59-76, wherein the second modified oligonucleotide comprises a conjugate group.

Embodiment 78. The oligomeric duplex of embodiment 77, wherein the conjugate group comprises a conjugate linker and a conjugate moiety.

Embodiment 79. The oligomeric duplex of embodiment 77 or embodiment 78, wherein the conjugate group is attached to the second modified oligonucleotide at the 5′-end of the second modified oligonucleotide.

Embodiment 80. The oligomeric duplex of embodiment 77 or embodiment 78, wherein the conjugate group is attached to the second modified oligonucleotide at the 3′-end of the modified oligonucleotide.

Embodiment 81. The oligomeric duplex of embodiment 77 or embodiment 78, wherein the conjugate group is attached via the 2′ position of a ribosyl sugar moiety at an internal position of the second modified oligonucleotide.

Embodiment 82. The oligomeric duplex of any of embodiments 77-81, wherein the conjugate group comprises a lipid.

Embodiment 83. The oligomeric duplex of any of embodiments 77-82, wherein the conjugate group comprises a cell-targeting moiety.

Embodiment 84. The oligomeric duplex of any of embodiments 77-83, wherein the second modified oligonucleotide comprises a terminal group.

Embodiment 85. The oligomeric duplex of embodiment 84, wherein the terminal group is an abasic sugar moiety.

Embodiment 86. The oligomeric duplex of any of embodiments 59-85, wherein the second modified oligonucleotide consists of 10 to 25, 10 to 30, 10 to 50, 12 to 20, 12 to 25, 12 to 30, 12 to 50, 13 to 20, 13 to 25, 13 to 30, 13 to 50, 14 to 20, 14 to 25, 14 to 30, 14 to 50, 15 to 20, 15 to 25, 15 to 30, 15 to 50, 16 to 18, 16 to 20, 16 to 25, 16 to 30, 16 to 50, 17 to 20, 17 to 25, 17 to 30, 17 to 50, 18 to 20, 18 to 22, 18 to 25, 18 to 30, 18 to 50, 19 to 20, 19 to 25, 19 to 30, 19 to 50, 20 to 25, 20 to 30, 20 to 50, 21 to 25, 21 to 30, 21 to 50, 22 to 25, 22 to 30, 22 to 50, 23 to 25, 23 to 30, or 23 to 50 linked nucleosides.

Embodiment 87. An antisense agent comprising an antisense compound, wherein the antisense compound is the oligomeric compound of any of embodiments 1-52.

Embodiment 88. An antisense agent, wherein the antisense agent is the oligomeric duplex of any of embodiments 59-86.

Embodiment 89. The antisense agent of embodiment 87 or embodiment 88, wherein the antisense agent is:
i) an RNase H agent capable of reducing the amount of PTBP1 nucleic acid through the activation of RNase H; or
ii) an RNAi agent capable of reducing the amount of PTBP1 nucleic acid through the activation of RISC/Ago2.

Embodiment 90. The antisense agent of any of embodiments 87-89, wherein the antisense agent comprises a conjugate group, and wherein the conjugate group comprises a cell-targeting moiety.

Embodiment 91. A pharmaceutical composition comprising an oligomeric compound of any of embodiments 1-52, a population of any of embodiments 53-58, an oligomeric duplex of any of embodiments 59-86, or an antisense agent of any of embodiments 87-90, and a pharmaceutically acceptable diluent or carrier.

Embodiment 92. The pharmaceutical composition of embodiment 91, wherein the pharmaceutically acceptable diluent is phosphate-buffered saline (PBS) or artificial cerebrospinal fluid (aCSF).

Embodiment 93. The pharmaceutical composition of embodiment 92, wherein the pharmaceutical composition consists essentially of the oligomeric compound of any of embodiments 1-52, the population of any of embodiments 53-58, the oligomeric duplex of any of embodiments 59-86, or the antisense agent of any of embodiments 87-90, and phosphate-buffered saline (PBS).

Embodiment 94. The pharmaceutical composition of embodiment 92, wherein the pharmaceutical composition consists essentially of the oligomeric compound of any of embodiments 1-52, the population of any of embodiments 53-58, the oligomeric duplex of any of embodiments 59-86, or the antisense agent of any of embodiments 87-90 and artificial cerebrospinal fluid (aCSF).

### Certain Oligomeric Agents and Oligomeric Compounds

Certain embodiments provide oligomeric agents targeted to a PTBP1 nucleic acid. In certain embodiments, the PTBP1 nucleic acid has the sequence set forth in GENBANK Accession No. NC_000019.10, truncated from nucleosides 794001 to 815000 (SEQ ID NO: 1) or GENBANK Accession No. NM_002819.4 (SEQ ID NO: 2), each of which is incorporated by reference in its entirety. In certain embodiments, the oligomeric agent is a single-stranded oligomeric compound. In certain embodiments, the oligomeric agent is an oligomeric duplex.

Certain embodiments provide an oligomeric compound comprising a modified oligonucleotide consisting of 8 to 80 linked nucleosides, wherein the nucleobase sequence of the modified oligonucleotide is at least 80% complementary to the nucleobase sequence of an equal length portion of a PTBP1 nucleic acid, and wherein the modified oligonucleotide has at least one modification selected from a modified sugar moiety and a modified internucleoside linkage. In certain embodiments, the PTBP1 nucleic acid has the nucleobase sequence of SEQ ID NOs: 1 or 2. In certain embodiments, the nucleobase sequence of the modified oligonucleotide is at least 85%, at least 90%, at least 95%, or 100% complementary to the nucleobase sequence of an equal length portion of the PTBP1 nucleic acid.

In certain embodiments, the nucleobase sequence of the modified oligonucleotide is at least 80% complementary to the nucleobase sequence of an equal length portion within nucleobases 4033-4052, 4034-4053, 4379-4398, 4381-4400, 4387-4406, 4388-4407, 4469-4488, 4483-4502, 4486-4505, 4815-4834, 4816-4835, 4845-4864, 4846-4865, 5117-5136, 5416-5435, 5417-5436, 5418-5437, 5569-5588, 5570-5589, 5571-5590, 5660-5679, 5661-5680, 5698-5717, 5747-5766, 5748-5767, 5807-5826, 5808-5827, 5809-5828, 5810-5829, 5811-5830, 5812-5831, 5813-5832, 5814-5833, 5833-5852, 5835-5854, 5838-5857, 5844-5863, 5845-5864, 5846-5865, 5870-5889, 6120-6139, 6127-6146, 6243-6262, 6252-6271, 6254-6273, 6256-6275, 6275-6294, 6288-6307, 6289-6308, 6291-6310, 6294-6313, 6296-6315, 6297-6316, 6298-6317, 6299-6318, 6301-6320, 6303-6322, 6304-6323, 6329-6348, 6330-6349, 6375-6394, 6378-6397, 6379-6398, 6380-6399, 6381-6400, 6382-6401, 6383-6402, 6386-6405, 6397-6416, 6398-6417, 6399-6418, 6401-6420, 6402-6421, 6404-6423, 6405-6424, 6406-6425, 6409-6428, 6461-6480, 6462-6481, 6466-6485, 6470-6489, 6587-6606, 6678-6697, 6705-6724, 6707-6726, 6708-6727, 6712-6731, 6713-6732, 6754-6773, 7107-7126, 7108-7127, 7109-7128, 7111-7130, 7245-7264, 7405-7424, 7441-7460, 7656-7675, 7657-7676, 7658-7677, 7661-7680, 7663-7682, 7692-7711, 7884-7903, 7890-7909, 7991-8010, 8103-8122, 8300-8319, 8323-8342, 8328-8347, 8338-8357, 8339-8358, 8431-8450, 8435-8454, 8449-8468, 8456-8475, 8457-8476, 8458-8477, 8459-8478, 8461-8480, 8462-8481, 8463-8482, 8467-8486, 8545-8564, 8550-8569, 8552-8571, 8557-8576, 8766-8785, 8787-8806, 8791-8810, 8792-8811, 8794-8813, 8815-8834, 8834-8853, 8835-8854, 8836-8855, 8846-8865, 8847-8866, 8851-8870, 8852-8871, 8862-8881, 8863-8882, 8864-8883, 8865-8884, 8866-8885, 8867-8886, 8868-8887, 8869-8888, 8870-8889, 8871-8890, 8872-8891, 8879-8898, 8880-8899, 8881-8900, 8884-8903, 8885-8904, 8886-8905, 8991-9010, 8992-9011, 9022-9041, 9026-9045, 9027-9046, 9051-9070, 9054-9073, 9253-9272, 9254-9273, 9255-9274, 9331-9350, 9335-9354, 9336-9355, 9337-9356, 9338-9357, 9339-9358, 9340-9359, 9341-9360, 9345-9364, 9422-9441, 9423-9442, 9424-9443, 9426-9445, 9430-9449, 9500-9519, 9521-9540, 9522-9541, 9573-9592, 9600-9619, 9602-9621, 9842-9861, 9843-9862, 9961-9980, 10037-10056, 10045-10064, 10047-10066, 10048-10067, 10057-10076, 10058-10077, 10128-10147, 10129-10148, 10131-10150, 10133-10152, 10134-10153, 10136-10155, 10159-10178, 10160-10179, 10306-10325, 10307-10326, 10308-10327, 10551-10570, 10679-10698, 10680-10699, 10844-10863, 10845-10864, 10847-10866, 10850-10869, 11445-11464, 11452-11471, 11459-11478, 11494-11513, 11745-11764, 11863-11882, 11866-11885, 11877-11896, 11880-11899, 12476-12495, 12478-12497, 12479-12498, 12507-12526, 12509-12528, 12511-12530, 12512-12531, 12513-12532, 12517-12536, 12518-12537, 12521-12540, 12522-12541, 12707-12726, 12782-12801, 12813-12832, 12814-12833, 12988-13007, 12990-13009, 13000-13019, 13351-13370, 13405-13424, 13406-13425, 13455-13474, 13457-13476, 13458-13477, 13462-13481, 13463-13482, 13464-13483, 13467-13486, 13468-13487, 13469-13488, 13470-13489, 13472-13491, 13858-13877, 13859-13878, 13941-13960, 13942-13961, 14039-14058, 14109-14128, 14236-14255, 14378-14397, 14379-14398, 14388-14407, 14393-14412, 14413-14432, 14417-14436, 14419-14438, 14633-14652, 14654-14673, 14796-14815, 14832-14851, 14834-14853, 14932-14951, 14934-14953, 14940-14959, 14984-15003, 14985-15004, 14986-15005, 14987-15006, 14988-15007, 14989-15008, 15380-15399, 15450-15469, 15675-15694, 15676-15695, 15730-15749, 15733-15752, 15735-15754, 15736-15755, 15737-15756, 15802-15821, 15803-15822, 15895-15914, 15896-15915, 15908-15927, 15912-15931, 15914-15933, 15915-15934, 15916-15935, 15918-15937, 15920-15939, 15921-15940, 15922-15941, 15990-16009, 15992-16011, 15995-16014, 15996-16015, 16000-16019, 16005-16024, 16301-16320, 16302-16321, 16304-16323, 16305-16324, 16308-16327, 16309-16328, 16310-16329, 16312-16331, 16313-16332, 16314-16333, 16315-16334, 16365-16384, 16387-16406, 16455-16474, 16456-16475, 16461-16480, 16550-16569, 16551-16570, 16553-16572, 16577-16596, 16593-16612, 16596-16615, 16602-16621, 16688-16707, 16698-16717, 16715-16734, 16731-16750, 16773-16792, 16872-16891, 16873-16892, 16885-16904, 16934-16953, 16935-16954, 16936-16955, 16937-16956, 16938-16957, 16940-16959, 16941-16960, 16964-16983, 16965-16984, 16967-16986, 17018-17037, 17019-17038, 17024-17043, 17025-17044, 17028-17047, 17097-17116, 17105-17124, 17210-17229, 17214-17233, 17215-17234, 17216-17235, 17217-17236, 17218-17237, 17231-17250, 17233-17252, 17236-17255, 17240-17259, 17241-17260, 17420-17439, 17429-17448, 17432-17451, 17434-17453, 17462-17481, 17463-17482, 17483-17502, 17484-17503, 17491-17510, 17503-17522, 17504-17523, 17513-17532, 17514-17533, 17515-17534, 17516-17535, 17518-17537, 17519-17538, 17520-17539, 17521-17540, 17522-17541, 17523-17542, 17529-17548, 17536-17555, 17537-17556, 17538-17557, 17545-17564, 17546-17565, 17547-17566, 17551-17570, 17552-17571, 17554-17573, 17555-17574, 17556-17575, 17557-17576, 17558-17577, 17560-17579, 17561-17580, 17562-17581, 17563-17582, 17564-17583, 17569-17588, 17594-17613, 17595-17614, 17623-17642, 17624-17643, 17625-17644, 17628-17647, 17655-17674, 17657-17676, 17725-17744, 17726-17745, 17730-17749, 17733-17752, 17734-17753, 17804-17823, 17858-17877, 17988-18007, 17990-18009, 17996-18015, 17997-18016, 17998-18017, 18002-18021, 18003-18022, 18004-18023, 18006-18025, 18008-18027, 18009-18028, 18010-18029, 18011-18030, 18035-18054, 18047-18066, 18062-18081, 18063-18082, 18065-18084, 18069-18088, 18080-18099, 18081-18100, 18085-18104, 18086-18105, 18087-18106, 18089-18108, 18109-18128, 18113-18132, 18194-18213, 18195-18214, 18205-18224, 18234-18253, 18256-18275, 18257-18276, 18260-18279, 18261-18280, 18286-18305, or 18287-18306 of SEQ ID NO: 1. In certain embodiments, the nucleobase sequence of the modified oligonucleotide is at least 85%, at least 90%, at least 95%, or 100% complementary to the equal length portion of the PTBP1 nucleic acid.

In certain embodiments, the nucleobase sequence of the modified oligonucleotide is at least 80% complementary to an equal length portion within nucleobases 16938-16960, 17538-17573, or 17988-18016 of SEQ ID NO: 1. In certain embodiments, the nucleobase sequence of the modified oligonucleotide is at least 85%, at least 90%, at least 95%, or 100% complementary to the equal length portion of the PTBP1 nucleic acid.

Certain embodiments provide an oligomeric compound comprising a modified oligonucleotide consisting of 8 to 80 linked nucleosides, wherein the nucleobase sequence of the modified oligonucleotide comprises at least 8, at least 9, at least 10, at least 11, at least 12, at least 13, at least 14, at least 15, at least 16, at least 17, at least 18, or at least 19 contiguous nucleobases of any of the nucleobase sequences of SEQ ID NOs: 21-483. In certain embodiments, the nucleobase sequence of the modified oligonucleotide comprises at least 10, at least 11, at least 12, at least 13, at least 14, at least 15, at least 16, at least 17, at least 18, at least 19, or 20 contiguous nucleobases of any of the nucleobase sequences of SEQ ID NOs: 21-483.

Certain embodiments provide an oligomeric compound comprising a modified oligonucleotide consisting of 20 to 80 linked nucleosides, wherein the nucleobase sequence of the modified oligonucleotide comprises the nucleobase sequence of any of nucleobase sequences of SEQ ID NOs: 21-483.

Certain embodiments provide an oligomeric compound comprising a modified oligonucleotide consisting of 20 linked nucleosides, wherein the modified oligonucleotide has a nucleobase sequence consisting of the nucleobase sequence of any of the nucleobase sequences of SEQ ID NOs: 21-483.

In any of the oligomeric compounds provided herein, the nucleobase sequence of the modified oligonucleotide can be at least 85%, at least 90%, at least 95%, or 100% complementary to the nucleobase sequence of an equal length portion of a PTBP1 nucleic acid, wherein the PTBP1 nucleic acid has the nucleobase sequence of SEQ ID NOs: 1 or 2.

In any of the oligomeric compounds provided herein, the modified oligonucleotide can consist of 10 to 25, 10 to 30, 10 to 50, 12 to 20, 12 to 25, 12 to 30, 12 to 50, 13 to 20, 13 to 25, 13 to 30, 13 to 50, 14 to 20, 14 to 25, 14 to 30, 14 to 50, 15 to 20, 15 to 25, 15 to 30, 15 to 50, 16 to 18, 16 to 20, 16 to 25, 16 to 30, 16 to 50, 17 to 20, 17 to 25, 17 to 30, 17 to 50, 18 to 20, 18 to 25, 18 to 30, 18 to 50, 19 to 20, 19 to 25, 19 to 30, 19 to 50, 20 to 25, 20 to 30, 20 to 50, 21 to 25, 21 to 30, 21 to 50, 22 to 25, 22 to 30, 22 to 50, 23 to 25, 23 to 30, or 23 to 50 linked nucleosides.

In certain embodiments, the modified oligonucleotide comprises at least 10, at least 11, at least 12, at least 13, at least 14, at least 15, at least 16, at least 17, at least 18, or at least 19 but no more than 50 linked nucleosides. In certain embodiments, the modified oligonucleotide consists of 16, 17, 18, 19, or 20 linked nucleosides.

In any of the oligomeric compounds provided herein, at least one nucleoside of the modified oligonucleotide can comprise a modified sugar moiety. In certain embodiments, the modified sugar moiety comprises a bicyclic sugar moiety, such as a 2′-4′ bridge selected from —O—CH2-; and —O—CH(CH3)-. In certain embodiments, the modified sugar moiety comprises a non-bicyclic modified sugar moiety, such as a 2′-MOE sugar moiety or a 2′-OMe sugar moiety.

In any of the oligomeric compounds provided herein, at least one nucleoside of the modified oligonucleotide compound can comprise a sugar surrogate.

In any of the oligomeric compounds provided herein, at least one internucleoside linkage of the modified oligonucleotide can comprise a modified internucleoside linkage, such as a phosphorothioate internucleoside linkage. In certain embodiments, each internucleoside linkage of the modified oligonucleotide can be a modified internucleoside linkage or each internucleoside linkage of the modified oligonucleotide can be a phosphorothioate internucleoside linkage. In certain embodiments, at least one internucleoside linkage of the modified oligonucleotide can be a phosphodiester internucleoside linkage. In certain embodiments, each internucleoside linkage of the modified oligonucleotide can be independently selected from a phosphodiester or a phosphorothioate internucleoside linkage. In certain embodiments, at least 2, at least 3, at least 4, at least 5, or at least 6 internucleoside linkages of the modified oligonucleotide can be phosphodiester internucleoside linkages. In certain embodiments, at least 10, at least 11, at least 12, at least 13, at least 14, at least 15, at least 16, at least 17, or at least 18 internucleoside linkages of the modified oligonucleotide can be phosphorothioate internucleoside linkages.

In any of the oligomeric compounds provided herein, at least one nucleobase of the modified oligonucleotide can be a modified nucleobase, such as 5-methylcytosine. In certain embodiments, each cytosine is 5-methylcytosine.

In any of the oligomeric compounds provided herein, the modified oligonucleotide can comprise a deoxy region consisting of 5-12 contiguous 2′-deoxynucleosides. In certain embodiments, each nucleoside of the deoxy region is a 2′-β-D-deoxynucleoside. In certain embodiments, the deoxy region consists of 7, 8, 9, 10, or 7-10 linked nucleosides. In certain embodiments, each nucleoside immediately adjacent to the deoxy region comprises a modified sugar moiety. In certain embodiments, the deoxy region is flanked on the 5′-side by a 5′ external region consisting of 1-6 linked 5′ external region nucleosides and on the 3′-side by a 3′external region consisting of 1-6 linked 3′external region nucleosides; wherein the 3′-most nucleoside of the 5′ external region comprises a modified sugar moiety; and the 5′-most nucleoside of the 3′external region comprises a modified sugar moiety. In certain embodiments, each nucleoside of the 3′ external region comprises a modified sugar moiety. In certain embodiments, each nucleoside of the 5′ external region comprises a modified sugar moiety.

### Certain Oligomeric Duplexes

Certain embodiments are directed to oligomeric duplexes comprising a first oligomeric compound and a second oligomeric compound.

In certain embodiments, an oligomeric duplex comprises:
a first oligomeric compound comprising a first modified oligonucleotide consisting of 8 to 80 linked nucleosides, wherein the nucleobase sequence of the first modified oligonucleotide is at least 80% complementary to the nucleobase sequence of an equal length portion within nucleobases 4033-4052, 4034-4053, 4379-4398, 4381-4400, 4387-4406, 4388-4407, 4469-4488, 4483-4502, 4486-4505, 4815-4834, 4816-4835, 4845-4864, 4846-4865, 5117-5136, 5416-5435, 5417-5436, 5418-5437, 5569-5588, 5570-5589, 5571-5590, 5660-5679, 5661-5680, 5698-5717, 5747-5766, 5748-5767, 5807-5826, 5808-5827, 5809-5828, 5810-5829, 5811-5830, 5812-5831, 5813-5832, 5814-5833, 5833-5852, 5835-5854, 5838-5857, 5844-5863, 5845-5864, 5846-5865, 5870-5889, 6120-6139, 6127-6146, 6243-6262, 6252-6271, 6254-6273, 6256-6275, 6275-6294, 6288-6307, 6289-6308, 6291-6310, 6294-6313, 6296-6315, 6297-6316, 6298-6317, 6299-6318, 6301-6320, 6303-6322, 6304-6323, 6329-6348, 6330-6349, 6375-6394, 6378-6397, 6379-6398, 6380-6399, 6381-6400, 6382-6401, 6383-6402, 6386-6405, 6397-6416, 6398-6417, 6399-6418, 6401-6420, 6402-6421, 6404-6423, 6405-6424, 6406-6425, 6409-6428, 6461-6480, 6462-6481, 6466-6485, 6470-6489, 6587-6606, 6678-6697, 6705-6724, 6707-6726, 6708-6727, 6712-6731, 6713-6732, 6754-6773, 7107-7126, 7108-7127, 7109-7128, 7111-7130, 7245-7264, 7405-7424, 7441-7460, 7656-7675, 7657-7676, 7658-7677, 7661-7680, 7663-7682, 7692-7711, 7884-7903, 7890-7909, 7991-8010, 8103-8122, 8300-8319, 8323-8342, 8328-8347, 8338-8357, 8339-8358, 8431-8450, 8435-8454, 8449-8468, 8456-8475, 8457-8476, 8458-8477, 8459-8478, 8461-8480, 8462-8481, 8463-8482, 8467-8486, 8545-8564, 8550-8569, 8552-8571, 8557-8576, 8766-8785, 8787-8806, 8791-8810, 8792-8811, 8794-8813, 8815-8834, 8834-8853, 8835-8854, 8836-8855, 8846-8865, 8847-8866, 8851-8870, 8852-8871, 8862-8881, 8863-8882, 8864-8883, 8865-8884, 8866-8885, 8867-8886, 8868-8887, 8869-8888, 8870-8889, 8871-8890, 8872-8891, 8879-8898, 8880-8899, 8881-8900, 8884-8903, 8885-8904, 8886-8905, 8991-9010, 8992-9011, 9022-9041, 9026-9045, 9027-9046, 9051-9070, 9054-9073, 9253-9272, 9254-9273, 9255-9274, 9331-9350, 9335-9354, 9336-9355, 9337-9356, 9338-9357, 9339-9358, 9340-9359, 9341-9360, 9345-9364, 9422-9441, 9423-9442, 9424-9443, 9426-9445, 9430-9449, 9500-9519, 9521-9540, 9522-9541, 9573-9592, 9600-9619, 9602-9621, 9842-9861, 9843-9862, 9961-9980, 10037-10056, 10045-10064, 10047-10066, 10048-10067, 10057-10076, 10058-10077, 10128-10147, 10129-10148, 10131-10150, 10133-10152, 10134-10153, 10136-10155, 10159-10178, 10160-10179, 10306-10325, 10307-10326, 10308-10327, 10551-10570, 10679-10698, 10680-10699, 10844-10863, 10845-10864, 10847-10866, 10850-10869, 11445-11464, 11452-11471, 11459-11478, 11494-11513, 11745-11764, 11863-11882, 11866-11885, 11877-11896, 11880-11899, 12476-12495, 12478-12497, 12479-12498, 12507-12526, 12509-12528, 12511-12530, 12512-12531, 12513-12532, 12517-12536, 12518-12537, 12521-12540, 12522-12541, 12707-12726, 12782-12801, 12813-12832, 12814-12833, 12988-13007, 12990-13009, 13000-13019, 13351-13370, 13405-13424, 13406-13425, 13455-13474, 13457-13476, 13458-13477, 13462-13481, 13463-13482, 13464-13483, 13467-13486, 13468-13487, 13469-13488, 13470-13489, 13472-13491, 13858-13877, 13859-13878, 13941-13960, 13942-13961, 14039-14058, 14109-14128, 14236-14255, 14378-14397, 14379-14398, 14388-14407, 14393-14412, 14413-14432, 14417-14436, 14419-14438, 14633-14652, 14654-14673, 14796-14815, 14832-14851, 14834-14853, 14932-14951, 14934-14953, 14940-14959, 14984-15003, 14985-15004, 14986-15005, 14987-15006, 14988-15007, 14989-15008, 15380-15399, 15450-15469, 15675-15694, 15676-15695, 15730-15749, 15733-15752, 15735-15754, 15736-15755, 15737-15756, 15802-15821, 15803-15822, 15895-15914, 15896-15915, 15908-15927, 15912-15931, 15914-15933, 15915-15934, 15916-15935, 15918-15937, 15920-15939, 15921-15940, 15922-15941, 15990-16009, 15992-16011, 15995-16014, 15996-16015, 16000-16019, 16005-16024, 16301-16320, 16302-16321, 16304-16323, 16305-16324, 16308-16327, 16309-16328, 16310-16329, 16312-16331, 16313-16332, 16314-16333, 16315-16334, 16365-16384, 16387-16406, 16455-16474, 16456-16475, 16461-16480, 16550-16569, 16551-16570, 16553-16572, 16577-16596, 16593-16612, 16596-16615, 16602-16621, 16688-16707, 16698-16717, 16715-16734, 16731-16750, 16773-16792, 16872-16891, 16873-16892, 16885-16904, 16934-16953, 16935-16954, 16936-16955, 16937-16956, 16938-16957, 16940-16959, 16941-16960, 16964-16983, 16965-16984, 16967-16986, 17018-17037, 17019-17038, 17024-17043, 17025-17044, 17028-17047, 17097-17116, 17105-17124, 17210-17229, 17214-17233, 17215-17234, 17216-17235, 17217-17236, 17218-17237, 17231-17250, 17233-17252, 17236-17255, 17240-17259, 17241-17260, 17420-17439, 17429-17448, 17432-17451, 17434-17453, 17462-17481, 17463-17482, 17483-17502, 17484-17503, 17491-17510, 17503-17522, 17504-17523, 17513-17532, 17514-17533, 17515-17534, 17516-17535, 17518-17537, 17519-17538, 17520-17539, 17521-17540, 17522-17541, 17523-17542, 17529-17548, 17536-17555, 17537-17556, 17538-17557, 17545-17564, 17546-17565, 17547-17566, 17551-17570, 17552-17571, 17554-17573, 17555-17574, 17556-17575, 17557-17576, 17558-17577, 17560-17579, 17561-17580, 17562-17581, 17563-17582, 17564-17583, 17569-17588, 17594-17613, 17595-17614, 17623-17642, 17624-17643, 17625-17644, 17628-17647, 17655-17674, 17657-17676, 17725-17744, 17726-17745, 17730-17749, 17733-17752, 17734-17753, 17804-17823, 17858-17877, 17988-18007, 17990-18009, 17996-18015, 17997-18016, 17998-18017, 18002-18021, 18003-18022, 18004-18023, 18006-18025, 18008-18027, 18009-18028, 18010-18029, 18011-18030, 18035-18054, 18047-18066, 18062-18081, 18063-18082, 18065-18084, 18069-18088, 18080-18099, 18081-18100, 18085-18104, 18086-18105, 18087-18106, 18089-18108, 18109-18128, 18113-18132, 18194-18213, 18195-18214, 18205-18224, 18234-18253, 18256-18275, 18257-18276, 18260-18279, 18261-18280, 18286-18305, or 18287-18306 of SEQ ID NO: 1; and
a second oligomeric compound comprising a second modified oligonucleotide consisting of 8 to 80 linked nucleosides wherein the nucleobase sequence of the second modified oligonucleotide comprises a complementary region of at least 8 nucleobases that is at least 90% complementary to the nucleobase sequence of an equal length portion of the first modified oligonucleotide. In certain embodiments, the nucleobase sequence of the first modified oligonucleotide is at least 85%, at least 90%, at least 95%, or 100% complementary to the nucleobase sequence of an equal length portion of the PTBP1 nucleic acid.

In certain embodiments, an oligomeric duplex comprises:
a first oligomeric compound comprising a first modified oligonucleotide consisting of 8 to 80 linked nucleosides, wherein the nucleobase sequence of the first modified oligonucleotide is at least 80% complementary to the nucleobase sequence of an equal length portion within nucleobases 16938-16960, 17538-17573, or 17988-18016 of SEQ ID NO: 1; and
a second oligomeric compound comprising a second modified oligonucleotide consisting of 8 to 80 linked nucleosides wherein the nucleobase sequence of the second modified oligonucleotide comprises a complementary region of at least 8 nucleobases that is at least 90% complementary to the nucleobase sequence of an equal length portion of the first modified oligonucleotide. In certain embodiments, the nucleobase sequence of the first modified oligonucleotide is at least 85%, at least 90%, at least 95%, or 100% complementary to the nucleobase sequence of an equal length portion of the PTBP1 nucleic acid.

In certain embodiments, an oligomeric duplex comprises:
a first oligomeric compound comprising a first modified oligonucleotide consisting of 8 to 80 linked nucleosides wherein the nucleobase sequence of the first modified oligonucleotide comprises at least 8, at least 9, at least 10, at least 11, at least 12, at least 13, at least 14, at least 15, at least 16, at least 17, at least 18, at least 19, or 20 contiguous nucleobases of the nucleobase sequence of any of SEQ ID NOs 21-483, wherein each thymine is replaced by uracil; and
a second oligomeric compound comprising a second modified oligonucleotide consisting of 8 to 80 linked nucleosides wherein the nucleobase sequence of the second modified oligonucleotide comprises a complementary region of at least 8 nucleobases that is at least 90% complementary to the nucleobase sequence of an equal length portion of the first modified oligonucleotide. In certain embodiments, the nucleobase sequence of the first modified oligonucleotide is at least 85%, at least 90%, at least 95%, or 100% complementary to the nucleobase sequence of an equal length portion of the PTBP1 nucleic acid.

In certain embodiments, the first oligomeric compound is an antisense compound. In certain embodiments, the first modified oligonucleotide is an antisense oligonucleotide. In certain embodiments, the second oligomeric compound is a sense compound. In certain embodiments, the second modified oligonucleotide is a sense oligonucleotide.

In certain embodiments, an oligomeric duplex comprises:
a first oligomeric compound comprising a first modified oligonucleotide consisting of 16 to 80 linked nucleosides wherein the nucleobase sequence of the first modified oligonucleotide comprises the nucleobase sequence of any of SEQ ID NOs 21-483, wherein each thymine is replaced by uracil; and
a second oligomeric compound comprising a second modified oligonucleotide consisting of 16 to 80 linked nucleosides wherein the nucleobase sequence of the second modified oligonucleotide comprises a complementary region of at least 16 nucleobases that is at least 90% complementary to the nucleobase sequence of an equal length portion of the first modified oligonucleotide.

In certain embodiments, the first oligomeric compound is an antisense compound. In certain embodiments, the first modified oligonucleotide is an antisense oligonucleotide. In certain embodiments, the second oligomeric compound is a sense compound. In certain embodiments, the second modified oligonucleotide is a sense oligonucleotide.

In any of the oligomeric duplexes described herein, at least one nucleoside of the first modified oligonucleotide and/or the second modified oligonucleotide can comprise a modified sugar moiety. Examples of suitable modified sugar moieties include, but are not limited to, a bicyclic sugar moiety, such as a 2′-4′ bridge selected from —O—CH2-; and —O—CH(CH3)-, and a non-bicyclic sugar moiety, such as a 2′-MOE sugar moiety, a 2′-F sugar moiety, a 2′-OMe sugar moiety, or a 2′-NMA sugar moiety. In certain embodiments, at least 80%, at least 90%, or 100% of the nucleosides of the first modified oligonucleotide and/or the second modified oligonucleotide comprises a modified sugar moiety selected from 2′-F and 2′-OMe.

In any of the oligomeric duplexes described herein, at least one nucleoside of the first modified oligonucleotide and/or the second modified oligonucleotide can comprise a sugar surrogate. Examples of suitable sugar surrogates include, but are not limited to, morpholino, peptide nucleic acid (PNA), glycol nucleic acid (GNA), and unlocked nucleic acid (UNA). In certain embodiments, at least one nucleoside of the first modified oligonucleotide comprises a sugar surrogate, which can be a GNA.

In any of the oligomeric duplexes described herein, at least one internucleoside linkage of the first modified oligonucleotide and/or the second modified oligonucleotide can comprise a modified internucleoside linkage. In certain embodiments, the modified internucleoside linkage is a phosphorothioate internucleoside linkage. In certain embodiments, at least one of the first, second, or third internucleoside linkages from the 5′ end and/or the 3′ end of the first modified oligonucleotide comprises a phosphorothioate linkage. In certain embodiments, at least one of the first, second, or third internucleoside linkages from the 5′ end and/or the 3′ end of the second modified oligonucleotide comprises a phosphorothioate linkage.

In any of the oligomeric duplexes described herein, at least one internucleoside linkage of the first modified oligonucleotide and/or the second modified oligonucleotide can comprise a phosphodiester internucleoside linkage.

In any of the oligomeric duplexes described herein, each internucleoside linkage of the first modified oligonucleotide and/or the second modified oligonucleotide can be independently selected from a phosphodiester or a phosphorothioate internucleoside linkage.

In any of the oligomeric duplexes described herein, at least one nucleobase of the first modified oligonucleotide and/or the second modified oligonucleotide can be modified nucleobase. In certain embodiments, the modified nucleobase is 5-methylcytosine.

In any of the oligomeric duplexes described herein, the first modified oligonucleotide can comprise a stabilized phosphate group attached to the 5′ position of the 5′-most nucleoside. In certain embodiments, the stabilized phosphate group comprises a cyclopropyl phosphonate or an (E)-vinyl phosphonate.

In any of the oligomeric duplexes described herein, the first modified oligonucleotide can comprise a conjugate group. In certain embodiments, the conjugate group comprises a conjugate linker and a conjugate moiety. In certain embodiments, the conjugate group is attached to the first modified oligonucleotide at the 5′-end of the first modified oligonucleotide. In certain embodiments, the conjugate group is attached to the first modified oligonucleotide at the 3′-end of the modified oligonucleotide. In certain embodiments, the conjugate group comprises N-acetyl galactosamine. In certain embodiments, the conjugate group comprises a cell-targeting moiety having an affinity for transferrin receptor (TfR), also known as TfR1 and CD71. In certain embodiments, the conjugate group comprises an anti-TfR1 antibody or fragment thereof. In certain embodiments, the conjugate group comprises a protein or peptide capable of binding TfR1. In certain embodiments, the conjugate group comprises an aptamer capable of binding TfR1. In certain embodiments, the conjugate group may comprise a conjugate moiety selected from any of a C22 alkyl, C20 alkyl, C16 alkyl, C10 alkyl, C21 alkyl, C19 alkyl, C18 alkyl, C15 alkyl, C14 alkyl, C13 alkyl, C12 alkyl, C11 alkyl, C9 alkyl, C8 alkyl, C7 alkyl, C6 alkyl, C5 alkyl, C22 alkenyl, C20 alkenyl, C16 alkenyl, C10 alkenyl, C21 alkenyl, C19 alkenyl, C18 alkenyl, C15 alkenyl, C14 alkenyl, C13 alkenyl, C12 alkenyl, C11 alkenyl, C9 alkenyl, C8 alkenyl, C7 alkenyl, C6 alkenyl, or C5 alkenyl. In certain embodiments, the conjugate group may comprise a conjugate moiety selected from any of a C22 alkyl, C20 alkyl, C16 alkyl, C10 alkyl, C21 alkyl, C19 alkyl, C18 alkyl, C15 alkyl, C14 alkyl, C13 alkyl, C12 alkyl, C11 alkyl, C9 alkyl, C8 alkyl, C7 alkyl, C6 alkyl, or C5 alkyl, where the alkyl chain has one or more unsaturated bonds.

In any of the oligomeric duplexes described herein, the second modified oligonucleotide can comprise a conjugate group. In certain embodiments, the conjugate group comprises a conjugate linker and a conjugate moiety. In certain embodiments, the conjugate group is attached to the second modified oligonucleotide at the 5′-end of the second modified oligonucleotide. In certain embodiments, the conjugate group is attached to the second modified oligonucleotide at the 3′-end of the modified oligonucleotide. In certain embodiments, the conjugate group comprises N-acetyl galactosamine. In certain embodiments, the conjugate group comprises a cell-targeting moiety having an affinity for transferrin receptor (TfR), also known as TfR1 and CD71. In certain embodiments, the conjugate group comprises an anti-TfR1 antibody or fragment thereof. In certain embodiments, the conjugate group comprises a protein or peptide capable of binding TfR1. In certain embodiments, the conjugate group comprises an aptamer capable of binding TfR1. In certain embodiments, the conjugate group may comprise a conjugate moiety selected from any of a C22 alkyl, C20 alkyl, C16 alkyl, C10 alkyl, C21 alkyl, C19 alkyl, C18 alkyl, C15 alkyl, C14 alkyl, C13 alkyl, C12 alkyl, CII alkyl, C9 alkyl, C8 alkyl, C7 alkyl, C6 alkyl, C5 alkyl, C22 alkenyl, C20 alkenyl, C16 alkenyl, C10 alkenyl, C21 alkenyl, C19 alkenyl, C18 alkenyl, C15 alkenyl, C14 alkenyl, C13 alkenyl, C12 alkenyl, C11 alkenyl, C9 alkenyl, C8 alkenyl, C7 alkenyl, C6 alkenyl, or C5 alkenyl. In certain embodiments, the conjugate group may comprise a conjugate moiety selected from any of a C22 alkyl, C20 alkyl, C16 alkyl, C10 alkyl, C21 alkyl, C19 alkyl, C18 alkyl, C15 alkyl, C14 alkyl, C13 alkyl, C12 alkyl, C11 alkyl, C9 alkyl, C8 alkyl, C7 alkyl, C6 alkyl, or C5 alkyl, where the alkyl chain has one or more unsaturated bonds.

In certain embodiments, an antisense agent comprises an antisense compound, which comprises an oligomeric compound or an oligomeric duplex described herein. In certain embodiments, an antisense agent, which can comprise an oligomeric compound or an oligomeric duplex described herein, is an RNAi agent capable of reducing the amount of PTBP1 nucleic acid through the activation of RISC/Ago2.

Certain embodiments provide an oligomeric agent comprising two or more oligomeric duplexes. In certain embodiments, an oligomeric agent comprises two or more of any of the oligomeric duplexes described herein. In certain embodiments, an oligomeric agent comprises two or more of the same oligomeric duplex, which can be any of the oligomeric duplexes described herein. In certain embodiments, the two or more oligomeric duplexes are linked together. In certain embodiments, the two or more oligomeric duplexes are covalently linked together. In certain embodiments, the second modified oligonucleotides of two or more oligomeric duplexes are covalently linked together. In certain embodiments, the second modified oligonucleotides of two or more oligomeric duplexes are covalently linked together at their 3′ ends. In certain embodiments, the two or more oligomeric duplexes are covalently linked together by a glycol linker, such as a tetraethylene glycol linker. Certain such compounds are described in, e.g., Alterman, et al., Nature Biotech., 37:844-894, 2019.

### I. Certain Oligonucleotides

In certain embodiments, provided herein are oligomeric compounds comprising oligonucleotides, which consist of linked nucleosides. Oligonucleotides may be unmodified oligonucleotides (RNA or DNA) or may be modified oligonucleotides. Modified oligonucleotides comprise at least one modification relative to unmodified RNA or DNA. That is, modified oligonucleotides comprise at least one modified nucleoside (comprising a modified sugar moiety and/or a modified nucleobase) and/or at least one modified internucleoside linkage. Certain modified nucleosides and modified internucleoside linkages suitable for use in modified oligonucleotides are described below.

### A. Certain Modified Nucleosides

Modified nucleosides comprise a modified sugar moiety or a modified nucleobase or both a modified sugar moiety and a modified nucleobase. In certain embodiments, modified nucleosides comprising the following modified sugar moieties and/or the following modified nucleobases may be incorporated into modified oligonucleotides.

### 1. Certain Sugar Moieties

In certain embodiments, modified sugar moieties are non-bicyclic modified sugar moieties. In certain embodiments, modified sugar moieties are bicyclic or tricyclic sugar moieties. In certain embodiments, modified sugar moieties are sugar surrogates. Such sugar surrogates may comprise one or more substitutions corresponding to those of other types of modified sugar moieties.

In certain embodiments, modified sugar moieties are non-bicyclic modified furanosyl sugar moieties comprising one or more acyclic substituent, including, but not limited to, substituents at the 2′, 3′, 4′, and/or 5′ positions. In certain embodiments, the furanosyl sugar moiety is a ribosyl sugar moiety. In certain embodiments, one or more acyclic substituent of non-bicyclic modified sugar moieties is branched.

In certain embodiments, non-bicyclic modifed sugar moieties comprise a substituent group at the 2′-position. Examples of substituent groups suitable for the 2′-position of modified sugar moieties include but are not limited to: —F, —OCH₃ (“OMe” or “O-methyl”), and —O(CH₂)₂OCH₃ (“MOE”). In certain embodiments, 2′-substituent groups are selected from among: halo, allyl, amino, azido, SH, CN, OCN, CF₃, OCF₃, O—C₁-C₁₀ alkoxy, O—C₁-C₁₀ substituted alkoxy, O—C₁-C₁₀ alkyl, O—C₁-C₁₀ substituted alkyl, S-alkyl, N(Rₘ)-alkyl, O-alkenyl, S-alkenyl, N(Rₘ)-alkenyl, O-alkynyl, S-alkynyl, N(Rₘ)-alkynyl, O-alkylenyl-O-alkyl, alkynyl, alkaryl, aralkyl, O-alkaryl, O-aralkyl, O(CH₂)₂SCH₃, O(CH₂)₂ON(Rₘ)(Rₙ) or OCH₂C(═O)—N(Rₘ)(Rₙ), where each Rₘ and Rₙ is, independently, H, an amino protecting group, or substituted or unsubstituted C₁-C₁₀ alkyl, —O(CH₂)₂ON(CH₃)₂ (“DMAOE”), 2′-O(CH₂)₂O(CH₂)₂N(CH₃)₂ (“DMAEOE”), and the 2′-substituent groups described in Cook et al., U.S. Pat. No. 6,531,584; Cook et al., U.S. Pat. No. 5,859,221; and Cook et al., U.S. Pat. No. 6,005,087. Certain embodiments of these 2′-substituent groups can be further substituted with one or more substituent groups independently selected from among: hydroxyl, amino, alkoxy, carboxy, benzyl, phenyl, nitro (NO₂), thiol, thioalkoxy, thioalkyl, halogen, alkyl, aryl, alkenyl and alkynyl.

In certain embodiments, a 2′-substituted non-bicyclic modified nucleoside comprises a sugar moiety comprising a non-bridging 2′-substituent group selected from: F, NH₂, N₃, OCF₃, OCH₃, O(CH₂)₃NH₂, CH₂CH═CH₂, OCH₂CH═CH₂, OCH₂CH₂OCH₃, O(CH₂)₂SCH₃, O(CH₂)₂ON(Rm)(Rn), O(CH₂)₂O(CH₂)₂N(CH₃)₂, and N-substituted acetamide (OCH₂C(═O)—N(Rₘ)(Rₙ)), where each Rₘ and Rₙ is, independently, H, an amino protecting group, or substituted or unsubstituted C₁-C₁₀ alkyl.

In certain embodiments, a 2′-substituted nucleoside non-bicyclic modified nucleoside comprises a sugar moiety comprising a non-bridging 2′-substituent group selected from: F, OCF₃, OCH₃, OCH₂CH₂OCH₃, O(CH₂)₂SCH₃, O(CH₂)₂ON(CH₃)₂, O(CH₂)₂O(CH₂)₂N(CH₃)₂, O(CH₂)₂ON(CH₃)₂ (“DMAOE”), O(CH₂)₂O(CH₂)₂N(CH₃)₂ (“DMAEOE”) and OCH₂C(═O)—N(H)CH₃ (“NMA”).

In certain embodiments, a 2′-substituted non-bicyclic modified nucleoside comprises a sugar moiety comprising a non-bridging 2′-substituent group selected from: F, OCH₃, OCH₂CH₂OCH₃, O(CH₂)₂SCH₃, O(CH₂)₂ON(CH₃)₂, O(CH₂)₂O(CH₂)₂N(CH₃)₂, and OCH₂C(═O)—N(H)CH₃ (“NMA”).

In certain embodiments, a 2′-substituted non-bicyclic modified nucleoside comprises a sugar moiety comprise[ng a non-bridging 2′-substituent group selected from: F, OCH₃, and OCH₂CH₂OCH₃.

In certain embodiments, modified furanosyl sugar moieties and nucleosides incorporating such modified furanosyl sugar moieties are further defined by isomeric configuration. For example, a 2′-deoxyfuranosyl sugar moiety may be in seven isomeric configurations other than the naturally occurring β-D-deoxyribosyl configuration. Such modified sugar moieties are described in, e.g., WO 2019/157531, incorporated by reference herein. A 2′-modified sugar moiety has an additional stereocenter at the 2′-position relative to a 2′-deoxyfuranosyl sugar moiety; therefore, such sugar moieties have a total of sixteen possible isomeric configurations. 2′-modified sugar moieties described herein are in the β-D-ribosyl isomeric configuration unless otherwise specified.

In certain embodiments, non-bicyclic modifed sugar moieties comprise a substituent group at the 4′-position. Examples of substituent groups suitable for the 4′-position of modified sugar moieties include but are not limited to alkoxy (e.g., methoxy), alkyl, and those described in Manoharan et al., WO2015/106128.

In certain embodiments, non-bicyclic modifed sugar moieties comprise a substituent group at the 3′-position. Examples of substituent groups suitable for the 3′-position of modified sugar moieties include, but are not limited to, alkoxy (e.g., methoxy) and alkyl (e.g., methyl, ethyl).

In certain embodiments, non-bicyclic modifed sugar moieties comprise a substituent group at the 5′-position. Examples of substituent groups suitable for the 5′-position of modified sugar moieties include, but are not limited to, vinyl, alkoxy (e.g., methoxy), and alkyl (e.g., methyl (R or S), ethyl).

In certain embodiments, non-bicyclic modified sugar moieties comprise more than one non-bridging sugar substituent, for example, 2′-F-5′-methyl sugar moieties and the modified sugar moieties and modified nucleosides described in Migawa et al., WO 2008/101157 and Rajeev et al., US2013/0203836).

In naturally occurring nucleic acids, sugars are linked to one another 3′ to 5′. In certain embodiments, oligonucleotides include one or more nucleoside or sugar moiety linked at an alternative position, for example at the 2′ position or inverted 5′ to 3′. For example, where the linkage is at the 2′ position, the 2′-substituent groups may instead be at the 3′-position.

Certain modified sugar moieties comprise a substituent that bridges two atoms of the furanosyl ring to form a second ring, resulting in a bicyclic sugar moiety. Nucleosides comprising such bicyclic sugar moieties have been referred to as bicyclic nucleosides (BNAs), locked nucleosides, or conformationally restricted nucleotides (CRN). Certain such compounds are described in US Patent Publication No. 2013/0190383; and PCT publication WO 2013/036868. In certain such embodiments, the bicyclic sugar moiety comprises a bridge between the 4′ and the 2′ furanose ring atoms. n certain such embodiments, the furanose ring is a ribose ring. Examples of such 4′ to 2′ bridging sugar substituents include but are not limited to: 4′-CH₂-2′, 4′-(CH₂)₂-2′, 4′-(CH₂)₃-2′, 4′-CH₂—O-2′ (“LNA”), 4′-CH₂—S-2′, 4′-(CH₂)₂—O-2′ (“ENA”), 4′-CH(CH₃)—O-2′ (referred to as “constrained ethyl” or “cEt” when in the S configuration), 4′-CH₂—O—CH₂-2′, 4′-CH₂—N(R)-2′, 4′-CH(CH₂OCH₃)—O-2′ (“constrained MOE” or “cMOE”) and analogs thereof (see, e.g., Seth et al., U.S. Pat. No. 7,399,845, Bhat et al., U.S. Pat. No. 7,569,686, Swayze et al., U.S. Pat. No. 7,741,457, and Swayze et al., U.S. Pat. No. 8,022,193), 4′-C(CH₃)(CH₃)—O-2′ and analogs thereof (see, e.g., Seth et al., U.S. Pat. No. 8,278,283), 4′-CH₂—N(OCH₃)-2′ and analogs thereof (see, e.g., Prakash et al., U.S. Pat. No. 8,278,425), 4′-CH₂—O—N(CH₃)-2′ (see, e.g., Allerson et al., U.S. Pat. No. 7,696,345 and Allerson et al., U.S. Pat. No. 8,124,745), 4′-CH₂—C(H)(CH₃)-2′ (see, e.g., Zhou, et al., J. Org. Chem., 2009, 74, 118-134), 4′-CH₂—C(═CH₂)-2′ and analogs thereof (see e.g., Seth et al., U.S. Pat. No. 8,278,426), 4′-C(RₐR_{b})—N(R)—O-2′, 4′-C(RₐR_{b})—O—N(R)-2′, 4′-CH₂—O—N(R)-2′, and 4′-CH₂—N(R)—O-2′, wherein each R, Rₐ, and R_{b} is, independently, H, a protecting group, or C₁-C₁₂ alkyl (see, e.g. Imanishi et al., U.S. Pat. No. 7,427,672).

In certain embodiments, such 4′ to 2′ bridges independently comprise from 1 to 4 linked groups independently selected from: —[C(Ra)(Rb)]n-, —[C(Ra)(Rb)]n-O—, C(Ra)═C(Rb)-, C(Ra)=N—, C(═NRa)-, —C(═O)—, —C(═S)—, —O—, —Si(Ra)₂-, —S(═O)x-, and N(Ra)-;
wherein:
x is 0, 1, or 2;
n is 1, 2, 3, or 4;
each Ra and Rb is, independently, H, a protecting group, hydroxyl, C1-C12 alkyl, substituted C1-C12 alkyl, C2-C12 alkenyl, substituted C2-C12 alkenyl, C2-C12 alkynyl, substituted C2-C12 alkynyl, C5-C20 aryl, substituted C5-C20 aryl, heterocycle radical, substituted heterocycle radical, heteroaryl, substituted heteroaryl, C5-C7 alicyclic radical, substituted C5-C7 alicyclic radical, halogen, OJ1, NJ1J2, SJ1, N3, COOJ1, acyl (C(═O)—H), substituted acyl, CN, sulfonyl (S(═O)2-J1), or sulfoxyl (S(═O)-J1); and
each J1 and J2 is, independently, H, C1-C12 alkyl, substituted C1-C12 alkyl, C2-C12 alkenyl, substituted C2-C12 alkenyl, C2-C12 alkynyl, substituted C2-C12 alkynyl, C5-C20 aryl, substituted C5-C20 aryl, acyl (C(═O)—H), substituted acyl, a heterocycle radical, a substituted heterocycle radical, C1-C12 aminoalkyl, substituted C1-C12 aminoalkyl, or a protecting group.

Additional bicyclic sugar moieties are known in the art, see, for example: Freier et al., Nucleic Acids Research, 1997, 25(22), 4429-4443, Albaek et al., J. Org. Chem., 2006, 71, 7731-7740, Singh et al., Chem. Commun., 1998, 4, 455-456; Koshkin et al., Tetrahedron, 1998, 54, 3607-3630; Wahlestedt et al., Proc. Natl. Acad. Sci. U.S.A, 2000, 97, 5633-5638; Kumar et al., Bioorg. Med. Chem. Lett., 1998, 8, 2219-2222; Singh et al., J. Org. Chem., 1998, 63, 10035-10039; Srivastava et al., J. Am. Chem. Soc., 2007, 129, 8362-8379; Elayadi et al., Curr. Opinion Invens. Drugs, 2001, 2, 558-561; Braasch et al., Chem. Biol., 2001, 8, 1-7; Orum et al., Curr. Opinion Mol. Ther., 2001, 3, 239-243; Wengel et al., U.S. Pat. No. 7,053,207, Imanishi et al., U.S. Pat. No. 6,268,490, Imanishi et al. U.S. Pat. No. 6,770,748, Imanishi et al., U.S. RE44,779; Wengel et al., U.S. Pat. No. 6,794,499, Wengel et al., U.S. Pat. No. 6,670,461; Wengel et al., U.S. Pat. No. 7,034,133, Wengel et al., U.S. Pat. No. 8,080,644; Wengel et al., U.S. Pat. No. 8,034,909; Wengel et al., U.S. Pat. No. 8,153,365; Wengel et al., U.S. Pat. No. 7,572,582; and Ramasamy et al., U.S. Pat. No. 6,525,191, Torsten et al., WO 2004/106356, Wengel et al., WO 1999/014226; Seth et al., WO 2007/134181; Seth et al., U.S. Pat. No. 7,547,684; Seth et al., U.S. Pat. No. 7,666,854; Seth et al., U.S. Pat. No. 8,088,746; Seth et al., U.S. Pat. No. 7,750,131; Seth et al., U.S. Pat. No. 8,030,467; Seth et al., U.S. Pat. No. 8,268,980; Seth et al., U.S. Pat. No. 8,546,556; Seth et al., U.S. Pat. No. 8,530,640; Migawa et al., U.S. Pat. No. 9,012,421; Seth et al., U.S. Pat. No. 8,501,805; Allerson et al., US2008/0039618; and Migawa et al., US2015/0191727.

In certain embodiments, bicyclic sugar moieties and nucleosides incorporating such bicyclic sugar moieties are further defined by isomeric configuration. For example, an LNA nucleoside (described herein) may be in the α-L configuration or in the β-D configuration.

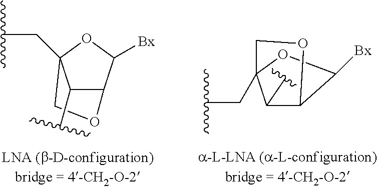

α-L-methyleneoxy (4′-CH₂—O-2′) or α-L-LNA bicyclic nucleosides have been incorporated into oligonucleotides that showed antisense activity (Frieden et al., Nucleic Acids Research, 2003, 21, 6365-6372). The addition of locked nucleic acids to siRNAs has been shown to increase siRNA stability in serum, and to reduce off-target effects (Elmen, J. et al., (2005) Nucleic Acids Research 33(1):439-447; Mook, O R. et al., (2007) Mal Cane Ther 6(3):833-843; Grunweller, A. et al., (2003) Nucleic Acids Research 31(12):3185-3193). Herein, general descriptions of bicyclic nucleosides include both isomeric configurations. When the positions of specific bicyclic nucleosides (e.g., LNA or cEt) are identified in exemplified embodiments herein, they are in the β-D configuration, unless otherwise specified.

In certain embodiments, modified sugar moieties comprise one or more non-bridging sugar substituent and one or more bridging sugar substituent (e.g., 5′-substituted and 4′-2′ bridged sugars).

In certain embodiments, modified sugar moieties are sugar surrogates. In certain such embodiments, the oxygen atom of the sugar moiety is replaced, e.g., with a sulfur, carbon, or nitrogen atom. In certain such embodiments, such modified sugar moieties also comprise bridging and/or non-bridging substituents as described herein. For example, certain sugar surrogates comprise a 4′-sulfur atom and a substitution at the 2′-position (see, e.g., Bhat et al., U.S. Pat. No. 7,875,733 and Bhat et al., U.S. Pat. No. 7,939,677) and/or the 5′ position.

In certain embodiments, sugar surrogates comprise rings having other than 5 atoms. For example, in certain embodiments, a sugar surrogate comprises a six-membered tetrahydropyran (“THP”). Such tetrahydropyrans may be further modified or substituted. Nucleosides comprising such modified tetrahydropyrans include but are not limited to hexitol nucleic acid (“HNA”), anitol nucleic acid (“ANA”), manitol nucleic acid (“MNA”) (see, e.g., Leumann, C J. Bioorg. & Med. Chem. 2002, 10, 841-854), fluoro HNA:

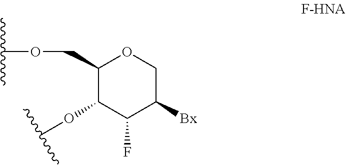

(“F-HNA”, see e.g. Swayze et al., U.S. Pat. No. 8,088,904; Swayze et al., U.S. Pat. No. 8,440,803; Swayze et al., U.S. Pat. No. 8,796,437; and Swayze et al., U.S. Pat. No. 9,005,906; F-HNA can also be referred to as a F-THP or 3′-fluoro tetrahydropyran), and nucleosides comprising additional modified THP compounds having the formula:

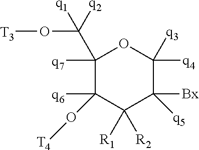

wherein, independently, for each of said modified THP nucleoside:
Bx is a nucleobase moiety;
T₃ and T₄ are each, independently, an internucleoside linking group linking the modified THP nucleoside to the remainder of an oligonucleotide or one of T₃ and T₄ is an internucleoside linking group linking the modified THP nucleoside to the remainder of an oligonucleotide and the other of T₃ and T₄ is H, a hydroxyl protecting group, a conjugate group or a 5′ or 3′-terminal group;
q₁, q₂, q₃, q₄, q₅, q₆ and q₇ are each, independently, H, C₁-C₆ alkyl, substituted C₁-C₆ alkyl, C₂-C₆ alkenyl, substituted C₂-C₆ alkenyl, C₂-C₆ alkynyl, or substituted C₂-C₆ alkynyl; and
each of R₁ and R₂ is independently selected from among: hydrogen, halogen, substituted or unsubstituted alkoxy, NJ₁J₂, SJ₁, N₃, OC(═X)J₁, OC(═X)NJ₁J₂, NJ₃C(═X)NJ₁J₂, and CN, wherein X is O, S or NJ₁, and
each J₁, J₂, and J₃ is, independently, H or C₁-C₆ alkyl.

In certain embodiments, modified THP nucleosides are provided wherein q₁, q₂, q₃, q₄, q₅, q₆ and q₇ are each H. In certain embodiments, at least one of q₁, q₂, q₃, q₄, q₅, q₆ and q₇ is other than H. In certain embodiments, at least one of q₁, q₂, q₃, q₄, q₅, q₆ and q₇ is methyl. In certain embodiments, modified THP nucleosides are provided wherein one of R₁ and R₂ is F. In certain embodiments, R₁ is F and R₂ is H, in certain embodiments, R₁ is methoxy and R₂ is H, and in certain embodiments, R₁ is methoxyethoxy and R₂ is H.

In certain embodiments, sugar surrogates comprise rings having more than 5 atoms and more than one heteroatom. For example, nucleosides comprising morpholino sugar moieties and their use in oligonucleotides have been reported (see, e.g., Braasch et al., Biochemistry, 2002, 41, 4503-4510 and Summerton et al., U.S. Pat. No. 5,698,685; Summerton et al., U.S. Pat. No. 5,166,315; Summerton et al., U.S. Pat. No. 5,185,444; and Summerton et al., U.S. Pat. No. 5,034,506). As used here, the term “morpholino” means a sugar surrogate having the following structure:

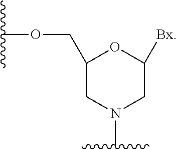

In certain embodiments, morpholinos may be modified, for example by adding or altering various substituent groups from the above morpholino structure. Such sugar surrogates are referred to herein as “modified morpholinos.”

In certain embodiments, sugar surrogates comprise acyclic moieties. Examples of nucleosides and oligonucleotides comprising such acyclic sugar surrogates include but are not limited to: peptide nucleic acid (“PNA”), acyclic butyl nucleic acid (see, e.g., Kumar et al., Org. Biomol. Chem., 2013, 11, 5853-5865), and nucleosides and oligonucleotides described in Manoharan et al., WO2011/133876. In certain embodiments, sugar surrogates comprise acyclic moieties. Examples of nucleosides and oligonucleotides comprising such acyclic sugar surrogates include, but are not limited to: peptide nucleic acid (“PNA”), acyclic butyl nucleic acid (see, e.g., Kumar et al., Org. Biomol. Chem., 2013, 11, 5853-5865), and nucleosides and oligonucleotides described in Manoharan et al., US2013/130378. Representative U.S. patents that teach the preparation of PNA compounds include, but are not limited to, U.S. Pat. Nos. 5,539,082; 5,714,331; and 5,719,262. Additional PNA compounds suitable for use in the oligonucleotides of the invention are described in, for example, in Nielsen et al., Science, 1991, 254, 1497-1500.

In certain embodiments, sugar surrogates are the “unlocked” sugar structure of UNA (unlocked nucleic acid) nucleosides. UNA is an unlocked acyclic nucleic acid, wherein any of the bonds of the sugar has been removed, forming an unlocked sugar surrogate. Representative U.S. publications that teach the preparation of UNA include, but are not limited to, U.S. Pat. No. 8,314,227; and US Patent Publication Nos. 2013/0096289; 2013/0011922; and 2011/0313020, the entire contents of each of which are hereby incorporated herein by reference.

In certain embodiments, sugar surrogates are the glycerol as found in GNA (glycol nucleic acid) nucleosides as depicted below:

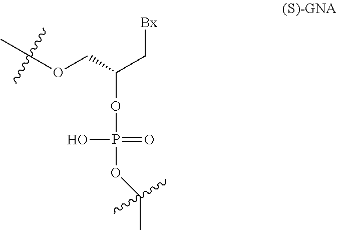

where Bx represents any nucleobase.

Many other bicyclic and tricyclic sugar and sugar surrogates are known in the art that can be used in modified nucleosides.

### 2. Certain Modified Nucleobases

In certain embodiments, modified oligonucleotides comprise one or more nucleosides comprising an unmodified nucleobase. In certain embodiments, modified oligonucleotides comprise one or more nucleosides comprising a modified nucleobase. In certain embodiments, modified oligonucleotides comprise one or more nucleosides that does not comprise a nucleobase, referred to as an abasic nucleoside. In certain embodiments, modified oligonucleotides comprise one or more inosine nucleosides (i.e., nucleosides comprising a hypoxanthine nucleobase).

In certain embodiments, modified nucleobases are selected from: 5-substituted pyrimidines, 6-azapyrimi-dines, alkyl or alkynyl substituted pyrimidines, alkyl substituted purines, and N-2, N-6 and O-6 substituted purines. In certain embodiments, modified nucleobases are selected from: 5-methylcytosine, 2-aminopropyladenine, 5-hydroxymethyl cytosine, xanthine, hypoxanthine, 2-aminoadenine, 6-N-methylguanine, 6-N-methyladenine, 2-propyladenine, 2-thiouracil, 2-thiothymine and 2-thiocytosine, 5-propynyl (—C═C—CH₃) uracil, 5-propynylcytosine, 6-azouracil, 6-azocytosine, 6-azothymine, 5-ribosyluracil (pseudouracil), 4-thiouracil, 8-halo, 8-amino, 8-thiol, 8-thioalkyl, 8-hydroxyl, 8-aza and other 8-substituted purines, 5-halo, particularly 5-bromo, 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6-N-benzoyladenine, 2-N-isobutyrylguanine, 4-N-benzoylcytosine, 4-N-benzoyluracil, 5-methyl 4-N-benzoylcytosine, 5-methyl 4-N-benzoyluracil, universal bases, hydrophobic bases, promiscuous bases, size-expanded bases, and fluorinated bases. Further modified nucleobases include tricyclic pyrimidines, such as 1,3-diazaphenoxazine-2-one, 1,3-diazaphenothiazine-2-one and 9-(2-aminoethoxy)-1,3-diazaphenoxazine-2-one (G-clamp). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in Merigan et al., U.S. Pat. No. 3,687,808, those disclosed in The Concise Encyclopedia Of Polymer Science And Engineering, Kroschwitz, J. I., Ed., John Wiley & Sons, 1990, 858-859; Englisch et al., Angewandte Chemie, International Edition, 1991, 30, 613; Sanghvi, Y. S., Chapter 15, Antisense Research and Applications, Crooke, S. T. and Lebleu, B., Eds., CRC Press, 1993, 273-288; and those disclosed in Chapters 6 and 15, Antisense Drug Technology, Crooke S. T., Ed., CRC Press, 2008, 163-166 and 442-443.

Publications that teach the preparation of certain of the above noted modified nucleobases as well as other modified nucleobases include without limitation, Manoharan et al., US2003/0158403; Manoharan et al., US2003/0175906; Dinh et al., U.S. Pat. No. 4,845,205; Spielvogel et al., U.S. Pat. No. 5,130,302; Rogers et al., U.S. Pat. No. 5,134,066; Bischofberger et al., U.S. Pat. No. 5,175,273; Urdea et al., U.S. Pat. No. 5,367,066; Benner et al., U.S. Pat. No. 5,432,272; Matteucci et al., U.S. Pat. No. 5,434,257; Gmeiner et al., U.S. Pat. No. 5,457,187; Cook et al., U.S. Pat. No. 5,459,255; Froehler et al., U.S. Pat. No. 5,484,908; Matteucci et al., U.S. Pat. No. 5,502,177; Hawkins et al., U.S. Pat. No. 5,525,711; Haralambidis et al., U.S. Pat. No. 5,552,540; Cook et al., U.S. Pat. No. 5,587,469; Froehler et al., U.S. Pat. No. 5,594,121; Switzer et al., U.S. Pat. No. 5,596,091; Cook et al., U.S. Pat. No. 5,614,617; Froehler et al., U.S. Pat. No. 5,645,985; Cook et al., U.S. Pat. No. 5,681,941; Cook et al., U.S. Pat. No. 5,811,534; Cook et al., U.S. Pat. No. 5,750,692; Cook et al., U.S. Pat. No. 5,948,903; Cook et al., U.S. Pat. No. 5,587,470; Cook et al., U.S. Pat. No. 5,457,191; Matteucci et al., U.S. Pat. No. 5,763,588; Froehler et al., U.S. Pat. No. 5,830,653; Cook et al., U.S. Pat. No. 5,808,027; Cook et al., U.S. Pat. No. 6,166,199; and Matteucci et al., U.S. Pat. No. 6,005,096.

### 3. Certain Modified Internucleoside Linkages

The naturally occurring internucleoside linkage of RNA and DNA is a 3′ to 5′ phosphodiester linkage. In certain embodiments, nucleosides of modified oligonucleotides may be linked together using one or more modified internucleoside linkages. The two main classes of internucleoside linking groups are defined by the presence or absence of a phosphorus atom. Representative phosphorus-containing internucleoside linkages include but are not limited to phosphates, which contain a phosphodiester bond (“P═O”) (also referred to as unmodified or naturally occurring linkages), phosphotriesters, methylphosphonates, phosphoramidates, and phosphorothioates (“P═S”), and phosphorodithioates (“HS—P═S”). Representative non-phosphorus containing internucleoside linking groups include but are not limited to methylenemethylimino (—CH₂—N(CH₃)—O—CH₂—), thiodiester, thionocarbamate (—O—C(═O)(NH)—S—); siloxane (—O—SiH₂—O—); and N,N′-dimethylhydrazine (—CH₂—N(CH₃)—N(CH₃)—). Modified internucleoside linkages, compared to naturally occurring phosphate linkages, can be used to alter, typically increase, nuclease resistance of the oligonucleotide. In certain embodiments, internucleoside linkages having a chiral atom can be prepared as a racemic mixture, or as separate enantiomers. Methods of preparation of phosphorous-containing and non-phosphorous-containing internucleoside linkages are well known to those skilled in the art.

In certain embodiments, a modified internucleoside linkage is any of those described in WO/2021/030778, incorporated by reference herein. In certain embodiments, a modified internucleoside linkage comprises the formula:

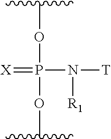

wherein independently for each internucleoside linking group of the modified oligonucleotide:
X is selected from O or S;
R₁ is selected from H, C₁-C₆ alkyl, and substituted C₁-C₆ alkyl; and
T is selected from SO₂R₂, C(═O)R₃, and P(═O)R₄R₅, wherein:
R₂ is selected from an aryl, a substituted aryl, a heterocycle, a substituted heterocycle, an aromatic heterocycle, a substituted aromatic heterocycle, a diazole, a substituted diazole, a C₁-C₆ alkoxy, C₁-C₆ alkyl, C₁-C₆ alkenyl, C₁-C₆ alkynyl, substituted C₁-C₆ alkyl, substituted C₁-C₆ alkenyl substituted C₁-C₆ alkynyl, and a conjugate group;
R₃ is selected from an aryl, a substituted aryl, CH₃, N(CH₃)₂, OCH₃ and a conjugate group;
R₄ is selected from OCH₃, OH, C₁-C₆ alkyl, substituted C₁-C₆ alkyl and a conjugate group; and
R₅ is selected from OCH₃, OH, C₁-C₆ alkyl, and substituted C₁-C₆ alkyl.

In certain embodiments, a modified internucleoside linkage comprises a mesyl phosphoramidate linking group having a formula:

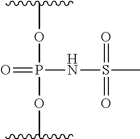 [SMILES: *OP(=O)(NS(C)(=O)=O)O*]

In certain embodiments, a mesyl phosphoramidate internucleoside linkage may comprise a chiral center. In certain embodiments, modified oligonucleotides comprising (Rp) and/or (Sp) mesyl phosphoramidates comprise one or more of the following formulas, respectively, wherein “B” indicates a nucleobase:

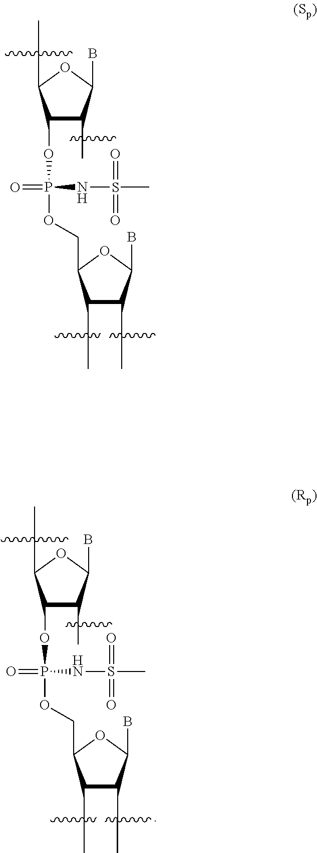

Representative internucleoside linkages having a chiral center include but are not limited to alkylphosphonates, mesyl phosphoramidates, and phosphorothioates. Modified oligonucleotides comprising internucleoside linkages having a chiral center can be prepared as populations of modified oligonucleotides comprising stereorandom internucleoside linkages, or as populations of modified oligonucleotides comprising phosphorothioate or other linkages containing chiral centers in particular stereochemical configurations. In certain embodiments, populations of modified oligonucleotides comprise phosphorothioate internucleoside linkages wherein all of the phosphorothioate internucleoside linkages are stereorandom. In certain embodiments, populations of modified oligonucleotides comprise mesyl phosphoramidate internucleoside linkages wherein all of the mesyl phosphoramidate internucleoside linkages are stereorandom. Such modified oligonucleotides can be generated using synthetic methods that result in random selection of the stereochemical configuration of each phosphorothioate or mesyl phosphoramidate linkage. Nonetheless, each individual phosphorothioate or mesyl phosphoramidate of each individual oligonucleotide molecule has a defined stereoconfiguration. In certain embodiments, populations of modified oligonucleotides are enriched for modified oligonucleotides comprising one or more particular phosphorothioate or mesyl phosphoramidate internucleoside linkages in a particular, independently selected stereochemical configuration. In certain embodiments, the particular configuration of the particular phosphorothioate or mesyl phosphoramidate linkage is present in at least 65% of the molecules in the population. In certain embodiments, the particular configuration of the particular phosphorothioate or mesyl phosphoramidate linkage is present in at least 70% of the molecules in the population. In certain embodiments, the particular configuration of the particular phosphorothioate or mesyl phosphoramidate linkage is present in at least 80% of the molecules in the population. In certain embodiments, the particular configuration of the particular phosphorothioate or mesyl phosphoramidate linkage is present in at least 90% of the molecules in the population. In certain embodiments, the particular configuration of the particular phosphorothioate or mesyl phosphoramidate linkage is present in at least 99% of the molecules in the population. Such chirally enriched populations of modified oligonucleotides can be generated using synthetic methods known in the art, e.g., methods described in Oka et al., JACCS 125, 8307 (2003), Wan et al. Nuc. Acid. Res. 42, 13456 (2014), and WO 2017/015555. In certain embodiments, a population of modified oligonucleotides is enriched for modified oligonucleotides having at least one indicated phosphorothioate or mesyl phosphoramidate in the (Sp) configuration. In certain embodiments, a population of modified oligonucleotides is enriched for modified oligonucleotides having at least one phosphorothioate or mesyl phosphoramidate in the (Rp) configuration. In certain embodiments, modified oligonucleotides comprising (Rp) and/or (Sp) phosphorothioates comprise one or more of the following formulas, respectively, wherein “B” indicates a nucleobase:

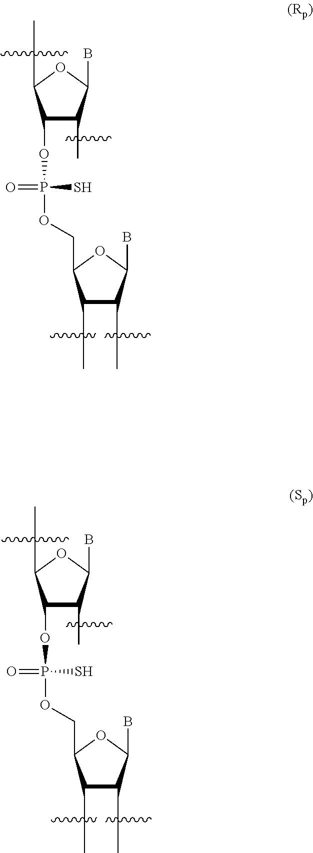

Unless otherwise indicated, chiral internucleoside linkages of modified oligonucleotides described herein can be stereorandom or in a particular stereochemical configuration.

Neutral internucleoside linkages include, without limitation, phosphotriesters, methylphosphonates, MMI (3′-CH₂—N(CH₃)—O-5′), amide-3 (3′-CH₂—C(═O)—N(H)-5′), amide-4 (3′-CH₂—N(H)—C(═O)-5′), formacetal (3′- O—CH₂—O-5′), methoxypropyl (MOP), and thioformacetal (3′-S—CH₂—O-5′). Further neutral internucleoside linkages include nonionic linkages comprising siloxane (dialkylsiloxane), carboxylate ester, carboxamide, sulfide, sulfonate ester and amides (See for example: Carbohydrate Modifications in Antisense Research; Y. S. Sanghvi and P. D. Cook, Eds., ACS Symposium Series 580; Chapters 3 and 4, 40-65). Further neutral internucleoside linkages include nonionic linkages comprising mixed N, O, S and CH₂ component parts.

In certain embodiments, modified oligonucleotides comprise one or more inverted nucleoside, as shown below:

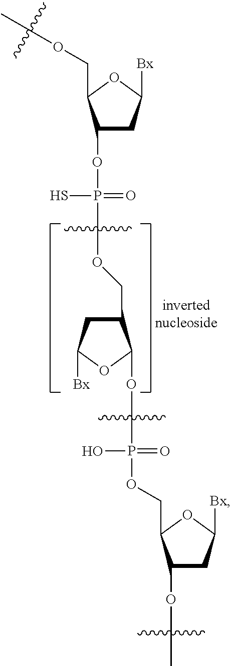

wherein each Bx independently represents any nucleobase.

In certain embodiments, an inverted nucleoside is terminal (i.e., the last nucleoside on one end of an oligonucleotide) and so only one internucleoside linkage depicted above will be present. In certain such embodiments, additional features (such as a conjugate group) may be attached to the inverted nucleoside. Such terminal inverted nucleosides can be attached to either or both ends of an oligonucleotide.

In certain embodiments, such groups lack a nucleobase and are referred to herein as inverted sugar moieties. In certain embodiments, an inverted sugar moiety is terminal (i.e., attached to the last nucleoside on one end of an oligonucleotide) and so only one internucleoside linkage above will be present. In certain such embodiments, additional features (such as a conjugate group) may be attached to the inverted sugar moiety. Such terminal inverted sugar moieties can be attached to either or both ends of an oligonucleotide.

In certain embodiments, nucleic acids can be linked 2′ to 5′ rather than the standard 3′ to 5′ linkage. Such a linkage is illustrated below.

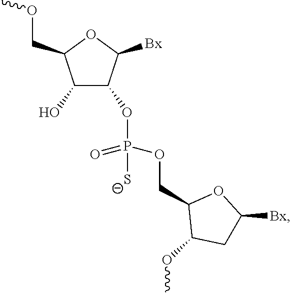

wherein each Bx represents any nucleobase.

### B. Certain Motifs

In certain embodiments, modified oligonucleotides comprise one or more modified nucleosides comprising a modified sugar moiety. In certain embodiments, modified oligonucleotides comprise one or more modified nucleosides comprising a modified nucleobase. In certain embodiments, modified oligonucleotides comprise one or more modified internucleoside linkage. In such embodiments, the modified, unmodified, and differently modified sugar moieties, nucleobases, and/or internucleoside linkages of a modified oligonucleotide define a pattern or motif. In certain embodiments, the patterns of sugar moieties, nucleobases, and internucleoside linkages are each independent of one another. Thus, a modified oligonucleotide may be described by its sugar motif, nucleobase motif and/or internucleoside linkage motif (as used herein, nucleobase motif describes the modifications to the nucleobases independent of the sequence of nucleobases).

### 1. Certain Sugar Motifs

In certain embodiments, oligonucleotides comprise one or more types of modified sugar and/or unmodified sugar moiety arranged along the oligonucleotide or region thereof in a defined pattern or sugar motif. In certain instances, such sugar motifs include but are not limited to any of the sugar modifications discussed herein.

In certain embodiments, modified oligonucleotides comprise or consist of a region having a gapmer motif, which is defined by two external regions or “wings” and a central or internal region or “gap.” The three regions of a gapmer motif (the 5′-wing, the gap, and the 3′-wing) form a contiguous sequence of nucleosides wherein at least some of the sugar moieties of the nucleosides of each of the wings differ from at least some of the sugar moieties of the nucleosides of the gap. Specifically, at least the sugar moieties of the nucleosides of each wing that are closest to the gap (the 3′-most nucleoside of the 5′-wing and the 5′-most nucleoside of the 3′-wing) differ from the sugar moiety of the neighboring gap nucleosides, thus defining the boundary between the wings and the gap (i.e., the wing/gap junction). In certain embodiments, the sugar moieties within the gap are the same as one another. In certain embodiments, the gap includes one or more nucleoside having a sugar moiety that differs from the sugar moiety of one or more other nucleosides of the gap. In certain embodiments, the sugar motifs of the two wings are the same as one another (symmetric gapmer). In certain embodiments, the sugar motif of the 5′-wing differs from the sugar motif of the 3′-wing (asymmetric gapmer).

In certain embodiments, the wings of a gapmer comprise 1-6 nucleosides. In certain embodiments, each nucleoside of each wing of a gapmer comprises a modified sugar moiety. In certain embodiments, at least one nucleoside of each wing of a gapmer comprises a modified sugar moiety. In certain embodiments, at least two nucleosides of each wing of a gapmer comprises a modified sugar moiety. In certain embodiments, at least three nucleosides of each wing of a gapmer comprises a modified sugar moiety. In certain embodiments, at least four nucleosides of each wing of a gapmer comprises a modified sugar moiety. In certain embodiments, at least five nucleosides of each wing of a gapmer comprises a modified sugar moiety.

In certain embodiments, the gap of a gapmer comprises 7-12 nucleosides. In certain embodiments, each nucleoside of the gap of a gapmer comprises a 2′-β-D-deoxyribosyl sugar moiety. In certain embodiments, at least one nucleoside of the gap of a gapmer comprises a modified sugar moiety.

In certain embodiments, the gapmer is a deoxy gapmer. In certain embodiments, the nucleosides on the gap side of each wing/gap junction comprise 2′-deoxyribosyl sugar moieties and the nucleosides on the wing sides of each wing/gap junction comprise modified sugar moieties. In certain embodiments, each nucleoside of the gap comprises a 2′-β-D-deoxyribosyl sugar moiety. In certain embodiments, each nucleoside of each wing of a gapmer comprises a modified sugar moiety. In certain embodiments, at least one nucleoside of the gap of a gapmer comprises a modified sugar moiety. In certain embodiments, one nucleoside of the gap comprises a modified sugar moiety and each remaining nucleoside of the gap comprises a 2′-deoxyribosyl sugar moiety. In certain embodiments, at least one nucleoside of the gap of a gapmer comprises a 2′-OMe sugar moiety.

In certain embodiments, modified oligonucleotides comprise or consist of a portion having a fully modified sugar motif. In such embodiments, each nucleoside of the fully modified portion of the modified oligonucleotide comprises a modified sugar moiety. In certain embodiments, each nucleoside of the entire modified oligonucleotide comprises a modified sugar moiety. In certain embodiments, modified oligonucleotides comprise or consist of a portion having a fully modified sugar motif, wherein each nucleoside within the fully modified portion comprises the same modified sugar moiety, referred to herein as a uniformly modified sugar motif. In certain embodiments, a fully modified oligonucleotide is a uniformly modified oligonucleotide. In certain embodiments, each nucleoside of a uniformly modified oligonucleotide comprises the same 2′-modification.

Herein, the lengths (number of nucleosides) of the three regions of a gapmer may be provided using the notation [#of nucleosides in the 5′-wing]-[#of nucleosides in the gap]-[#of nucleosides in the 3′-wing]. Thus, a 3-10-3 gapmer consists of 3 linked nucleosides in each wing and 10 linked nucleosides in the gap. Where such nomenclature is followed by a specific modification, that modification is the modification in each sugar moiety of each wing and the gap nucleosides comprise 2′-β-D-deoxyribosyl sugar moieties. Thus, a 5-10-5 MOE gapmer consists of 5 linked 2′-MOE nucleosides in the 5′-wing, 10 linked 2′-β-D-deoxynucleosides in the gap, and 5 linked 2′-MOE nucleosides in the 3′-wing. A 6-10-4 MOE gapmer consists of 6 linked 2′-MOE nucleosides in the 5′-wing, 10 linked 2′-β-D-deoxynucleosides in the gap, and 4 linked 2′-MOE nucleosides in the 3′-wing. A 3-10-3 cEt gapmer consists of 3 linked cEt nucleosides in the 5′-wing, 10 linked 2′-β-D-deoxynucleosides in the gap, and 3 linked cEt nucleosides in the 3′-wing.

In certain embodiments, modified oligonucleotides are 5-10-5 MOE gapmers. In certain embodiments, modified oligonucleotides are 6-10-4 MOE gapmers. In certain embodiments, modified oligonucleotides are 3-10-3 BNA gapmers. In certain embodiments, modified oligonucleotides are 3-10-3 cEt gapmers. In certain embodiments, modified oligonucleotides are 3-10-3 LNA gapmers.

In certain embodiments, modified oligonucleotides have a sugar motif selected from: 5′-eeeeeddddddddddeeeee-3′; wherein each “d” represents a 2′-β-D-deoxyribosyl sugar moiety, and each “e” represents a 2′-MOE sugar moiety.

In certain embodiments, modified oligonucleotides have the sugar motif from: 5′-eeeeedyddddddddeeeee-3′; wherein each “d” represents a 2′-β-D-deoxyribosyl sugar moiety, each “e” represents a 2′-MOE sugar moiety, and each “y” represents a 2′-OMe sugar moiety.

### 2. Certain Nucleobase Motifs

In certain embodiments, oligonucleotides comprise modified and/or unmodified nucleobases arranged along the oligonucleotide or region thereof in a defined pattern or motif. In certain embodiments, each nucleobase is modified. In certain embodiments, none of the nucleobases are modified. In certain embodiments, each purine or each pyrimidine is modified. In certain embodiments, each adenine is modified. In certain embodiments, each guanine is modified. In certain embodiments, each thymine is modified. In certain embodiments, each uracil is modified. In certain embodiments, each cytosine is modified. In certain embodiments, some or all of the cytosine nucleobases in a modified oligonucleotide are 5-methylcytosines. In certain embodiments, all of the cytosine nucleobases are 5-methylcytosines and all of the other nucleobases of the modified oligonucleotide are unmodified nucleobases.

In certain embodiments, modified oligonucleotides comprise a block of modified nucleobases. In certain such embodiments, the block is at the 3′-end of the oligonucleotide. In certain embodiments the block is within 3 nucleosides of the 3′-end of the oligonucleotide. In certain embodiments, the block is at the 5′-end of the oligonucleotide. In certain embodiments, the block is within 3 nucleosides of the 5′-end of the oligonucleotide.

In certain embodiments, oligonucleotides having a gapmer motif comprise a nucleoside comprising a modified nucleobase. In certain such embodiments, one nucleoside comprising a modified nucleobase is in the central gap of an oligonucleotide having a gapmer motif. In certain such embodiments, the sugar moiety of said nucleoside is a 2′-deoxyribosyl sugar moiety. In certain embodiments, the modified nucleobase is selected from a 2-thiopyrimidine and a 5-propynepyrimidine.

### 3. Certain Internucleoside Linkage Motifs

In certain embodiments, oligonucleotides comprise modified and/or unmodified internucleoside linkages arranged along the oligonucleotide or region thereof in a defined pattern or motif. In certain embodiments, each internucleoside linking group is a phosphodiester internucleoside linkage (P═O). In certain embodiments, each internucleoside linking group of a modified oligonucleotide is a phosphorothioate internucleoside linkage (P═S). In certain embodiments, each internucleoside linkage of a modified oligonucleotide is independently selected from a phosphorothioate internucleoside linkage and phosphodiester internucleoside linkage. In certain embodiments, each phosphorothioate internucleoside linkage is independently selected from a stereorandom phosphorothioate, a (Sp) phosphorothioate, and a (Rp) phosphorothioate.

In certain embodiments, the sugar motif of a modified oligonucleotide is a gapmer and the internucleoside linkages within the gap are all modified. In certain such embodiments, some or all of the internucleoside linkages in the wings are unmodified phosphodiester internucleoside linkages. In certain embodiments, the terminal internucleoside linkages are modified. In certain embodiments, the sugar motif of a modified oligonucleotide is a gapmer, and the internucleoside linkage motif comprises at least one phosphodiester internucleoside linkage in at least one wing, wherein the at least one phosphodiester linkage is not a terminal internucleoside linkage, and the remaining internucleoside linkages are phosphorothioate internucleoside linkages. In certain such embodiments, all of the phosphorothioate linkages are stereorandom. In certain embodiments, all of the phosphorothioate linkages in the wings are (Sp) phosphorothioates, and the gap comprises at least one Sp, Sp, Rp motif. In certain embodiments, populations of modified oligonucleotides are enriched for modified oligonucleotides comprising such internucleoside linkage motifs.

In certain embodiments, modified oligonucleotides have an internucleoside linkage motif of 5′-sooosssssssssssooss-3′, wherein each “s” represents a phosphorothioate internucleoside linkage and each “o” represents a phosphodiester internucleoside linkage.

In certain embodiments, modified oligonucleotides have an internucleoside linkage motif comprising one or more mesyl phosphoramidate linking groups. In certain embodiments, one or more phosphorothioate internucleoside linkages or one or more phosphodiester internucleoside linkages of the internucleoside linkage motifs herein is substituted with a mesyl phosphoramidate linking group.

### C. Certain Lengths

It is possible to increase or decrease the length of an oligonucleotide without eliminating activity. For example, in Woolf et al. (Proc. Natl. Acad. Sci. USA 89:7305-7309, 1992), a series of oligonucleotides 13-25 nucleobases in length were tested for their ability to induce cleavage of a target RNA in an oocyte injection model. Oligonucleotides 25 nucleobases in length with 8 or 11 mismatch bases near the ends of the oligonucleotides were able to direct specific cleavage of the target RNA, albeit to a lesser extent than the oligonucleotides that contained no mismatches. Similarly, target specific cleavage was achieved using 13 nucleobase oligonucleotides, including those with 1 or 3 mismatches.

In certain embodiments, oligonucleotides (including modified oligonucleotides) can have any of a variety of ranges of lengths. In certain embodiments, oligonucleotides consist of X to Y linked nucleosides, where X represents the fewest number of nucleosides in the range and Y represents the largest number nucleosides in the range. In certain such embodiments, X and Y are each independently selected from 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50; provided that X≤Y. For example, in certain embodiments, oligonucleotides consist of 12 to 13, 12 to 14, 12 to 15, 12 to 16, 12 to 17, 12 to 18, 12 to 19, 12 to 20, 12 to 21, 12 to 22, 12 to 23, 12 to 24, 12 to 25, 12 to 26, 12 to 27, 12 to 28, 12 to 29, 12 to 30, 13 to 14, 13 to 15, 13 to 16, 13 to 17, 13 to 18, 13 to 19, 13 to 20, 13 to 21, 13 to 22, 13 to 23, 13 to 24, 13 to 25, 13 to 26, 13 to 27, 13 to 28, 13 to 29, 13 to 30, 14 to 15, 14 to 16, 14 to 17, 14 to 18, 14 to 19, 14 to 20, 14 to 21, 14 to 22, 14 to 23, 14 to 24, 14 to 25, 14 to 26, 14 to 27, 14 to 28, 14 to 29, 14 to 30, 15 to 16, 15 to 17, 15 to 18, 15 to 19, 15 to 20, 15 to 21, 15 to 22, 15 to 23, 15 to 24, 15 to 25, 15 to 26, 15 to 27, 15 to 28, 15 to 29, 15 to 30, 16 to 17, 16 to 18, 16 to 19, 16 to 20, 16 to 21, 16 to 22, 16 to 23, 16 to 24, 16 to 25, 16 to 26, 16 to 27, 16 to 28, 16 to 29, 16 to 30, 17 to 18, 17 to 19, 17 to 20, 17 to 21, 17 to 22, 17 to 23, 17 to 24, 17 to 25, 17 to 26, 17 to 27, 17 to 28, 17 to 29, 17 to 30, 18 to 19, 18 to 20, 18 to 21, 18 to 22, 18 to 23, 18 to 24, 18 to 25, 18 to 26, 18 to 27, 18 to 28, 18 to 29, 18 to 30, 19 to 20, 19 to 21, 19 to 22, 19 to 23, 19 to 24, 19 to 25, 19 to 26, 19 to 29, 19 to 28, 19 to 29, 19 to 30, 20 to 21, 20 to 22, 20 to 23, 20 to 24, 20 to 25, 20 to 26, 20 to 27, 20 to 28, 20 to 29, 20 to 30, 21 to 22, 21 to 23, 21 to 24, 21 to 25, 21 to 26, 21 to 27, 21 to 28, 21 to 29, 21 to 30, 22 to 23, 22 to 24, 22 to 25, 22 to 26, 22 to 27, 22 to 28, 22 to 29, 22 to 30, 23 to 24, 23 to 25, 23 to 26, 23 to 27, 23 to 28, 23 to 29, 23 to 30, 24 to 25, 24 to 26, 24 to 27, 24 to 28, 24 to 29, 24 to 30, 25 to 26, 25 to 27, 25 to 28, 25 to 29, 25 to 30, 26 to 27, 26 to 28, 26 to 29, 26 to 30, 27 to 28, 27 to 29, 27 to 30, 28 to 29, 28 to 30, or 29 to 30 linked nucleosides.

In certain embodiments, oligonucleotides (including modified oligonucleotides) consist of 16 linked nucleosides. In certain embodiments, oligonucleotides (including modified oligonucleotides) consist of 17 linked nucleosides. In certain embodiments, oligonucleotides (including modified oligonucleotides) consist of 18 linked nucleosides. In certain embodiments, oligonucleotides (including modified oligonucleotides) consist of 19 linked nucleosides. In certain embodiments, oligonucleotides (including modified oligonucleotides) consist of 20 linked nucleosides.

### D. Certain Modified Oligonucleotides

In certain embodiments, the above modifications (sugar, nucleobase, internucleoside linkage) are incorporated into a modified oligonucleotide. In certain embodiments, modified oligonucleotides are characterized by their modification motifs and overall lengths. In certain embodiments, such parameters are each independent of one another. Thus, unless otherwise indicated, each internucleoside linkage of an oligonucleotide having a gapmer sugar motif may be modified or unmodified and may or may not follow the gapmer modification pattern of the sugar modifications. For example, the internucleoside linkages within the wing regions of a sugar gapmer may be the same or different from one another and may be the same or different from the internucleoside linkages of the gap region of the sugar motif. Likewise, such sugar gapmer oligonucleotides may comprise one or more modified nucleobase independent of the gapmer pattern of the sugar modifications. Unless otherwise indicated, all modifications are independent of nucleobase sequence.

### E. Certain Populations of Modified Oligonucleotides

Populations of modified oligonucleotides in which all of the modified oligonucleotides of the population have the same molecular formula can be stereorandom populations or chirally enriched populations. All of the chiral centers of all of the modified oligonucleotides are stereorandom in a stereorandom population. In a chirally enriched population, at least one particular chiral center is not stereorandom in the modified oligonucleotides of the population. In certain embodiments, the modified oligonucleotides of a chirally enriched population are enriched for β-D ribosyl sugar moieties, and all of the phosphorothioate internucleoside linkages are stereorandom. In certain embodiments, the modified oligonucleotides of a chirally enriched population are enriched for both β-D ribosyl sugar moieties and at least one, particular phosphorothioate internucleoside linkage in a particular stereochemical configuration.

### F. Nucleobase Sequence

In certain embodiments, oligonucleotides (unmodified or modified oligonucleotides) are further described by their nucleobase sequence. In certain embodiments oligonucleotides have a nucleobase sequence that is complementary to a second oligonucleotide or an identified reference nucleic acid, such as a target nucleic acid. In certain such embodiments, a region of an oligonucleotide has a nucleobase sequence that is complementary to a second oligonucleotide or an identified reference nucleic acid, such as a target nucleic acid. In certain embodiments, the nucleobase sequence of a region or entire length of an oligonucleotide is at least 50%, at least 60%, at least 70%, at least 80%, at least 85%, at least 90%, at least 95%, or 100% complementary to the second oligonucleotide or nucleic acid, such as a target nucleic acid.

### II. Certain Oligomeric Compounds

In certain embodiments, provided herein are oligomeric compounds, which consist of an oligonucleotide (modified or unmodified) and optionally one or more conjugate groups and/or terminal groups. Conjugate groups consist of one or more conjugate moiety and a conjugate linker which links the conjugate moiety to the oligonucleotide. Conjugate groups may be attached to either or both ends of an oligonucleotide and/or at any internal position. In certain embodiments, conjugate groups are attached to the 2′-position of a nucleoside of a modified oligonucleotide. In certain embodiments, conjugate groups that are attached to either or both ends of an oligonucleotide are terminal groups. In certain such embodiments, conjugate groups or terminal groups are attached at the 3′ and/or 5′-end of oligonucleotides. In certain such embodiments, conjugate groups (or terminal groups) are attached at the 3′-end of oligonucleotides. In certain embodiments, conjugate groups are attached near the 3′-end of oligonucleotides. In certain embodiments, conjugate groups (or terminal groups) are attached at the 5′-end of oligonucleotides. In certain embodiments, conjugate groups are attached near the 5′-end of oligonucleotides.

Examples of terminal groups include but are not limited to conjugate groups, capping groups, phosphate moieties, protecting groups, modified or unmodified nucleosides, and two or more nucleosides that are independently modified or unmodified.

### A. Certain Conjugate Groups

In certain embodiments, oligonucleotides are covalently attached to one or more conjugate groups. In certain embodiments, conjugate groups modify one or more properties of the attached oligonucleotide, including but not limited to pharmacodynamics, pharmacokinetics, stability, binding, absorption, tissue distribution, cellular distribution, cellular uptake, charge, and clearance.

In certain embodiments, conjugation of one or more carbohydrate moieties to a modified oligonucleotide can optimize one or more properties of the modified oligonucleotide. In certain embodiments, the carbohydrate moiety is attached to a modified subunit of the modified oligonucleotide. For example, the ribose sugar of one or more ribonucleotide subunits of a modified oligonucleotide can be replaced with another moiety, e.g. a non-carbohydrate (preferably cyclic) carrier to which is attached a carbohydrate ligand. A ribonucleotide subunit in which the ribose sugar of the subunit has been so replaced is referred to herein as a ribose replacement modification subunit (RRMS), which is a modified sugar moiety. A cyclic carrier may be a carbocyclic ring system, i.e., one or more ring atoms may be a heteroatom, e.g., nitrogen, oxygen, sulphur. The cyclic carrier may be a monocyclic ring system, or may contain two or more rings, e.g. fused rings. The cyclic carrier may be a fully saturated ring system, or it may contain one or more double bonds. In certain embodiments, the modified oligonucleotide is a gapmer.

In certain embodiments, conjugate groups impart a new property on the attached oligonucleotide, e.g., fluorophores or reporter groups that enable detection of the oligonucleotide. Certain conjugate groups and conjugate moieties have been described previously, for example: cholesterol moiety (Letsinger et al., Proc. Natl. Acad. Sci. USA, 1989, 86, 6553-6556), cholic acid (Manoharan et al., Bioorg. Med. Chem. Lett., 1994, 4, 1053-1060), a thioether, e.g., hexyl-S-tritylthiol (Manoharan et al., Ann. N.Y. Acad. Sci., 1992, 660, 306-309; Manoharan et al., Bioorg. Med. Chem. Lett., 1993, 3, 2765-2770), a thiocholesterol (Oberhauser et al., Nucl. Acids Res., 1992, 20, 533-538), an aliphatic chain, e.g., do-decan-diol or undecyl residues (Saison-Behmoaras et al., EMBO J, 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethyl-ammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid a palmityl moiety (Mishra et al., Biochim. Biophys. Acta, 1995, 1264, 229-237), an octadecylamine or hexylamino-carbonyl-oxycholesterol moiety (Crooke et al., J Pharmacol. Exp. Ther., 1996, 277, 923-937), a tocopherol group (Nishina et al., Molecular Therapy Nucleic Acids, 2015, 4, e220; and Nishina et al., Molecular Therapy, 2008, 16, 734-740), or a GalNAc cluster (e.g., WO2014/179620).

In certain embodiments, the conjugate group may comprise a conjugate moiety selected from any of a C22 alkyl, C20 alkyl, C16 alkyl, C10 alkyl, C21 alkyl, C19 alkyl, C18 alkyl, C15 alkyl, C14 alkyl, C13 alkyl, C12 alkyl, C11 alkyl, C9 alkyl, C8 alkyl, C7 alkyl, C6 alkyl, C5 alkyl, C22 alkenyl, C20 alkenyl, C16 alkenyl, C10 alkenyl, C21 alkenyl, C19 alkenyl, C18 alkenyl, C15 alkenyl, C14 alkenyl, C13 alkenyl, C12 alkenyl, C11 alkenyl, C9 alkenyl, C8 alkenyl, C7 alkenyl, C6 alkenyl, or C5 alkenyl.

In certain embodiments, the conjugate group may comprise a conjugate moiety selected from any of a C22 alkyl, C20 alkyl, C16 alkyl, C10 alkyl, C21 alkyl, C19 alkyl, C18 alkyl, C15 alkyl, C14 alkyl, C13 alkyl, C12 alkyl, C11 alkyl, C9 alkyl, C8 alkyl, C7 alkyl, C6 alkyl, or C5 alkyl, where the alkyl chain has one or more unsaturated bonds.

In certain embodiments, a conjugate group is lipid having the following structure:

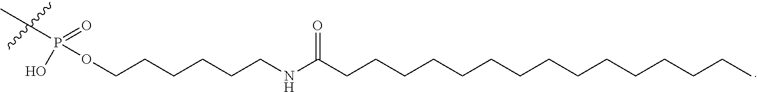 [SMILES: *P(=O)(O)OCCCCCCNC(=O)CCCCCCCCCCCCCCC]

### 1. Conjugate Moieties

Conjugate moieties include, without limitation, intercalators, reporter molecules, polyamines, polyamides, peptides, carbohydrates (e.g., GalNAc), antibodies, vitamin moieties, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins, fluorophores, and dyes.

In certain embodiments, a conjugate moiety comprises an active drug substance, for example, aspirin, warfarin, phenylbutazone, ibuprofen, suprofen, fen-bufen, ketoprofen, (S)-(+)-pranoprofen, carprofen, dansylsarcosine, 2,3,5-triiodobenzoic acid, fingolimod, flufenamic acid, folinic acid, a benzothiadiazide, chlorothiazide, a diazepine, indo-methicin, a barbiturate, a cephalosporin, a sulfa drug, an antidiabetic, an antibacterial or an antibiotic.

### 2. Conjugate Linkers

Conjugate moieties are attached to oligonucleotides through conjugate linkers. In certain oligomeric compounds, the conjugate linker is a single chemical bond (i.e., the conjugate moiety is attached directly to an oligonucleotide through a single bond). In certain embodiments, the conjugate linker comprises a chain structure, such as a hydrocarbyl chain, or an oligomer of repeating units such as ethylene glycol, nucleosides, or amino acid units.

In certain embodiments, a conjugate linker comprises pyrrolidine.

In certain embodiments, a conjugate linker comprises one or more groups selected from alkyl, amino, oxo, amide, disulfide, polyethylene glycol, ether, thioether, and hydroxylamino. In certain such embodiments, the conjugate linker comprises groups selected from alkyl, amino, oxo, amide and ether groups. In certain embodiments, the conjugate linker comprises groups selected from alkyl and amide groups. In certain embodiments, the conjugate linker comprises groups selected from alkyl and ether groups. In certain embodiments, the conjugate linker comprises at least one phosphorus moiety. In certain embodiments, the conjugate linker comprises at least one phosphate group. In certain embodiments, the conjugate linker includes at least one neutral linking group.

In certain embodiments, conjugate linkers, including the conjugate linkers described above, are bifunctional linking moieties, e.g., those known in the art to be useful for attaching conjugate moieties to compounds, such as the oligonucleotides provided herein. In general, a bifunctional linking moiety comprises at least two functional groups. One of the functional groups is selected to react with a particular site on a compound and the other is selected to react with a conjugate moiety. Examples of functional groups used in a bifunctional linking moiety include but are not limited to electrophiles for reacting with nucleophilic groups and nucleophiles for reacting with electrophilic groups. In certain embodiments, bifunctional linking moieties comprise one or more groups selected from amino, hydroxyl, carboxylic acid, thiol, alkyl, alkenyl, and alkynyl.

Examples of conjugate linkers include but are not limited to pyrrolidine, 8-amino-3,6-dioxaoctanoic acid (ADO), succinimidyl 4-(N-maleimidomethyl) cyclohexane-1-carboxylate (SMCC) and 6-aminohexanoic acid (AHEX or AHA). Other conjugate linkers include but are not limited to substituted or unsubstituted C₁-C₁₀ alkyl, substituted or unsubstituted C₂-C₁₀ alkenyl or substituted or unsubstituted C₂-C₁₀ alkynyl, wherein a nonlimiting list of preferred substituent groups includes hydroxyl, amino, alkoxy, carboxy, benzyl, phenyl, nitro, thiol, thioalkoxy, halogen, alkyl, aryl, alkenyl and alkynyl.

In certain embodiments, conjugate linkers comprise 1-10 linker-nucleosides. In certain embodiments, conjugate linkers comprise 2-5 linker-nucleosides. In certain embodiments, conjugate linkers comprise exactly 3 linker-nucleosides. In certain embodiments, conjugate linkers comprise the TCA motif. In certain embodiments, such linker-nucleosides are modified nucleosides. In certain embodiments such linker-nucleosides comprise a modified sugar moiety. In certain embodiments, linker-nucleosides are unmodified. In certain embodiments, linker-nucleosides comprise an optionally protected heterocyclic base selected from a purine, substituted purine, pyrimidine or substituted pyrimidine. In certain embodiments, a cleavable moiety is a nucleoside selected from uracil, thymine, cytosine, 4-N-benzoylcytosine, 5-methylcytosine, 4-N-benzoyl-5-methyl cytosine, adenine, 6-N-benzoyladenine, guanine and 2-N-isobutyrylguanine. It is typically desirable for linker-nucleosides to be cleaved from the oligomeric compound after it reaches a target tissue. Accordingly, linker-nucleosides are typically linked to one another and to the remainder of the oligomeric compound through cleavable bonds. In certain embodiments, such cleavable bonds are phosphodiester bonds.

Herein, linker-nucleosides are not considered to be part of the oligonucleotide. Accordingly, in embodiments in which an oligomeric compound comprises an oligonucleotide consisting of a specified number or range of linked nucleosides and/or a specified percent complementarity to a reference nucleic acid and the oligomeric compound also comprises a conjugate group comprising a conjugate linker comprising linker-nucleosides, those linker-nucleosides are not counted toward the length of the oligonucleotide and are not used in determining the percent complementarity of the oligonucleotide for the reference nucleic acid. For example, an oligomeric compound may comprise (1) a modified oligonucleotide consisting of 8-30 nucleosides and (2) a conjugate group comprising 1-10 linker-nucleosides that are contiguous with the nucleosides of the modified oligonucleotide. The total number of contiguous linked nucleosides in such an oligomeric compound is more than 30. Alternatively, an oligomeric compound may comprise a modified oligonucleotide consisting of 8-30 nucleosides and no conjugate group. The total number of contiguous linked nucleosides in such an oligomeric compound is no more than 30. Unless otherwise indicated conjugate linkers comprise no more than 10 linker-nucleosides. In certain embodiments, conjugate linkers comprise no more than 5 linker-nucleosides. In certain embodiments, conjugate linkers comprise no more than 3 linker-nucleosides. In certain embodiments, conjugate linkers comprise no more than 2 linker-nucleosides. In certain embodiments, conjugate linkers comprise no more than 1 linker-nucleoside.

In certain embodiments, it is desirable for a conjugate group to be cleaved from the oligonucleotide. For example, in certain circumstances oligomeric compounds comprising a particular conjugate moiety are better taken up by a particular cell type, but once the oligomeric compound has been taken up, it is desirable that the conjugate group be cleaved to release the unconjugated or parent oligonucleotide. Thus, certain conjugate linkers may comprise one or more cleavable moieties. In certain embodiments, a cleavable moiety is a cleavable bond. In certain embodiments, a cleavable moiety is a group of atoms comprising at least one cleavable bond. In certain embodiments, a cleavable moiety comprises a group of atoms having one, two, three, four, or more than four cleavable bonds. In certain embodiments, a cleavable moiety is selectively cleaved inside a cell or subcellular compartment, such as a lysosome. In certain embodiments, a cleavable moiety is selectively cleaved by endogenous enzymes, such as nucleases.

In certain embodiments, a cleavable bond is selected from among: an amide, an ester, an ether, one or both esters of a phosphodiester, a phosphate ester, a carbamate, or a disulfide. In certain embodiments, a cleavable bond is one or both of the esters of a phosphodiester. In certain embodiments, a cleavable moiety comprises a phosphate or phosphodiester. In certain embodiments, the cleavable moiety is a phosphate linkage between an oligonucleotide and a conjugate moiety or conjugate group.

In certain embodiments, a cleavable moiety comprises or consists of one or more linker-nucleosides. In certain such embodiments, the one or more linker-nucleosides are linked to one another and/or to the remainder of the oligomeric compound through cleavable bonds. In certain embodiments, such cleavable bonds are unmodified phosphodiester bonds. In certain embodiments, a cleavable moiety is 2′-deoxynucleoside that is attached to either the 3′ or 5-terminal nucleoside of an oligonucleotide by a phosphate internucleoside linkage and covalently attached to the remainder of the conjugate linker or conjugate moiety by a phosphate or phosphorothioate linkage. In certain such embodiments, the cleavable moiety is 2′-deoxyadenosine.

### 3. Cell-Targeting Moieties

In certain embodiments, a conjugate group comprises a cell-targeting moiety. In certain embodiments, a conjugate group has the general formula:

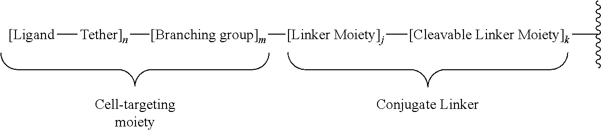

wherein n is from 1 to about 3, m is 0 when n is 1, m is 1 when n is 2 or greater, j is 1 or 0, and k is 1 or 0.

In certain embodiments, n is 1, j is 1 and k is 0. In certain embodiments, n is 1, j is 0 and k is 1. In certain embodiments, n is 1, j is 1 and k is 1. In certain embodiments, n is 2, j is 1 and k is 0. In certain embodiments, n is 2, j is 0 and k is 1. In certain embodiments, n is 2, j is 1 and k is 1. In certain embodiments, n is 3, j is 1 and k is 0. In certain embodiments, n is 3, j is 0 and k is 1. In certain embodiments, n is 3, j is 1 and k is 1.

In certain embodiments, conjugate groups comprise cell-targeting moieties that have at least one tethered ligand. In certain embodiments, cell-targeting moieties comprise two tethered ligands covalently attached to a branching group.

In certain embodiments, each ligand of a cell-targeting moiety has an affinity for at least one type of receptor on a target cell. In certain embodiments, each ligand has an affinity for at least one type of receptor on the surface of a mammalian liver cell. In certain embodiments, each ligand has an affinity for the hepatic asialoglycoprotein receptor (ASGP-R). In certain embodiments, each ligand is a carbohydrate.

In certain embodiments, a conjugate group comprises a cell-targeting conjugate moiety. In certain embodiments, a conjugate group has the general formula:

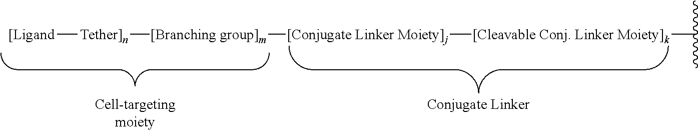

wherein n is from 1 to about 3, m is 0 when n is 1, m is 1 when n is 2 or greater, j is 1 or 0, and k is 1 or 0.

In certain embodiments, n is 1, j is 1 and k is 0. In certain embodiments, n is 1, j is 0 and k is 1. In certain embodiments, n is 1, j is 1 and k is 1. In certain embodiments, n is 2, j is 1 and k is 0. In certain embodiments, n is 2, j is 0 and k is 1. In certain embodiments, n is 2, j is 1 and k is 1. In certain embodiments, n is 3, j is 1 and k is 0. In certain embodiments, n is 3, j is 0 and k is 1. In certain embodiments, n is 3, j is 1 and k is 1.

In certain embodiments, conjugate groups comprise cell-targeting moieties that have at least one tethered ligand. In certain embodiments, cell-targeting moieties comprise two tethered ligands covalently attached to a branching group. In certain embodiments, cell-targeting moieties comprise three tethered ligands covalently attached to a branching group. In certain embodiments, the cell-targeting moiety targets neurons. In certain embodiments, the cell-targeting moiety targets a neurotransmitter receptor. In certain embodiments, the cell targeting moiety targets a neurotransmitter transporter. In certain embodiments, the cell targeting moiety targets a GABA transporter. See e.g., WO 2011/131693, WO 2014/064257.

In certain embodiments, conjugate groups comprise cell-targeting moieties that have affinities for transferrin receptor (TfR) (also referred to herein as TfR1 and CD71). In certain embodiments, a conjugate group described herein comprises an anti-TfR1 antibody or fragment thereof. In certain embodiments, the conjugate group comprises a protein or peptide capable of binding TfR1. In certain embodiments, the conjugate group comprises an aptamer capable of binding TfR1. In certain embodiments, the anti-TfR1 antibody or fragment thereof can be any known in the art including but not limited to those described in WO1991/004753; WO2013/103800; WO2014/144060; WO2016/081643; WO2016/179257; WO2016/207240; WO2017/221883; WO2018/129384; WO2018/124121; WO2019/151539; WO2020/132584; WO2020/028864; U.S. Pat. Nos. 7,208,174; 9,034,329; and 10,550,188. In certain embodiments, a fragment of an anti-TfR1 antibody is F(ab′)2, Fab, Fab′, Fv, or scFv.

In certain embodiments, the conjugate group comprises a protein or peptide capable of binding TfR1. In certain embodiments, the protein or peptide capable of binding TfR1 can be any known in the art including but not limited to those described in WO2019/140050; WO2020/037150; WO2020/124032; and U.S. Pat. No. 10,138,483. In certain embodiments, the conjugate group comprises an aptamer capable of binding TfR1. In certain embodiments, the aptamer capable of binding TfR1 can be any known in the art including but not limited to those described in WO2013/163303; WO2019/033051; and WO2020/245198.

### B. Certain Terminal Groups

In certain embodiments, oligomeric compounds comprise one or more terminal groups. In certain such embodiments, oligomeric compounds comprise a stabilized 5′-phosphate. Stabilized 5′-phosphates include, but are not limited to 5′-phosphonates, including, but not limited to 5′-vinylphosphonates. In certain embodiments, terminal groups comprise one or more abasic sugar moieties and/or inverted nucleosides. In certain embodiments, terminal groups comprise one or more 2′-linked nucleosides or sugar moieties. In certain such embodiments, the 2′-linked group is an abasic sugar moiety.

### III. Antisense Activity

In certain embodiments, oligomeric compounds and oligomeric duplexes are capable of hybridizing to a target nucleic acid, resulting in at least one antisense activity; such oligomeric compounds and oligomeric duplexes are antisense compounds. In certain embodiments, antisense compounds have antisense activity when they reduce or inhibit the amount or activity of a target nucleic acid by 25% or more in the standard cell assay. In certain embodiments, antisense compounds selectively affect one or more target nucleic acid. Such antisense compounds comprise a nucleobase sequence that hybridizes to one or more target nucleic acid, resulting in one or more desired antisense activity and does not hybridize to one or more non-target nucleic acid or does not hybridize to one or more non-target nucleic acid in such a way that results in significant undesired antisense activity.

In certain antisense activities, hybridization of an antisense compound to a target nucleic acid results in recruitment of a protein that cleaves the target nucleic acid. For example, certain antisense compounds result in RNase H mediated cleavage of the target nucleic acid. RNase H is a cellular endonuclease that cleaves the RNA strand of an RNA:DNA duplex. The DNA in such an RNA:DNA duplex need not be unmodified DNA. In certain embodiments, described herein are antisense compounds that are sufficiently “DNA-like” to elicit RNase H activity. In certain embodiments, one or more non-DNA-like nucleoside in the gap of a gapmer is tolerated.

In certain antisense activities, an antisense compound or a portion of an antisense compound is loaded into an RNA-induced silencing complex (RISC), ultimately resulting in cleavage of the target nucleic acid. For example, certain antisense compounds result in cleavage of the target nucleic acid by Argonaute. Antisense compounds that are loaded into RISC are RNAi compounds. RNAi compounds may be double-stranded (siRNA or dsRNAi) or single-stranded (ssRNA).

In certain embodiments, hybridization of an antisense compound to a target nucleic acid does not result in recruitment of a protein that cleaves that target nucleic acid. In certain embodiments, hybridization of the antisense compound to the target nucleic acid results in alteration of splicing of the target nucleic acid. In certain embodiments, hybridization of an antisense compound to a target nucleic acid results in inhibition of a binding interaction between the target nucleic acid and a protein or other nucleic acid. In certain embodiments, hybridization of an antisense compound to a target nucleic acid results in alteration of translation of the target nucleic acid.

Antisense activities may be observed directly or indirectly. In certain embodiments, observation or detection of an antisense activity involves observation or detection of a change in an amount of a target nucleic acid or protein encoded by such target nucleic acid, a change in the ratio of splice variants of a nucleic acid or protein and/or a phenotypic change in a cell or animal.

### IV. Certain Target Nucleic Acids

In certain embodiments, oligomeric agents, oligomeric compounds, oligomeric duplexes, or antisense agents comprise an oligonucleotide comprising a region that is complementary to a target nucleic acid. In certain embodiments, the target nucleic acid is an endogenous RNA molecule. In certain embodiments, the target nucleic acid encodes a protein. In certain such embodiments, the target nucleic acid is selected from: a mature mRNA and a pre-mRNA, including intronic, exonic and untranslated regions. In certain embodiments, the target RNA is a mature mRNA. In certain embodiments, the target nucleic acid is a pre-mRNA. In certain embodiments, the target region is entirely within an intron. In certain embodiments, the target region spans an intron/exon junction. In certain embodiments, the target region is at least 50% within an intron. In certain embodiments, the target nucleic acid is the RNA transcriptional product of a retrogene. In certain embodiments, the target nucleic acid is a non-coding RNA. In certain embodiments, the target non-coding RNA is selected from: a long non-coding RNA, a short non-coding RNA, and an intronic RNA molecule.

### A. Complementarity/Mismatches to the Target Nucleic Acid and Duplex Complementarity

In certain embodiments, oligonucleotides are complementary to the target nucleic acid over the entire length of the oligonucleotide. In certain embodiments, oligonucleotides are 99%, 95%, 90%, 85%, or 80% complementary to the target nucleic acid. In certain embodiments, oligonucleotides are at least 80% complementary to the target nucleic acid over the entire length of the oligonucleotide and comprise a region that is 100% or fully complementary to a target nucleic acid. In certain embodiments, the region of full complementarity is from 6 to 20, 10 to 18, or 18 to 20 nucleobases in length.

It is possible to introduce mismatch bases without eliminating activity. For example, Gautschi et al (J. Natl. Cancer Inst. 93:463-471, March 2001) demonstrated the ability of an oligonucleotide having 100% complementarity to the bcl-2 mRNA and having 3 mismatches to the bcl-xL mRNA to reduce the expression of both bcl-2 and bcl-xL in vitro and in vivo. Furthermore, this oligonucleotide demonstrated potent anti-tumor activity in vivo. Maher and Dolnick (Nuc. Acid. Res. 16:3341-3358, 1988) tested a series of tandem 14 nucleobase oligonucleotides, and 28 and 42 nucleobase oligonucleotides comprised of the sequence of two or three of the tandem oligonucleotides, respectively, for their ability to arrest translation of human DHFR in a rabbit reticulocyte assay. Each of the three 14 nucleobase oligonucleotides alone was able to inhibit translation, albeit at a more modest level than the 28 or 42 nucleobase oligonucleotides.

In certain embodiments, oligonucleotides comprise one or more mismatched nucleobases relative to the target nucleic acid. In certain embodiments, antisense activity against the target is reduced by such mismatch, but activity against a non-target is reduced by a greater amount. Thus, in certain embodiments selectivity of the oligonucleotide is improved.

In certain embodiments, a mismatch is specifically positioned within an oligonucleotide having a gapmer motif. In certain embodiments, the mismatch is at position 1, 2, 3, 4, 5, 6, 7, or 8 from the 5′-end of the gap region. In certain embodiments, the mismatch is at position 9, 8, 7, 6, 5, 4, 3, 2, 1 from the 3′-end of the gap region. In certain embodiments, the mismatch is at position 1, 2, 3, or 4 from the 5′-end of the wing region. In certain embodiments, the mismatch is at position 4, 3, 2, or 1 from the 3′-end of the wing region.

### B. PTBP1

In certain embodiments, oligomeric agents, oligomeric compounds, oligomeric duplexes, or antisense agents comprise a modified oligonucleotide comprising a region that is complementary to a target nucleic acid, wherein the target nucleic acid is a PTBP1 nucleic acid. In certain embodiments, a PTBP1 nucleic acid has the sequence set forth in SEQ ID NO: 1 (GENBANK Accession No. NC_000019.10, truncated from nucleosides 794001 to 815000) or SEQ ID NO: 2 (GENBANK Accession No. NM_002819.4). In certain embodiments, contacting a cell with an oligomeric agent, an oligomeric compound, an oligomeric duplex, or an antisense agent complementary to SEQ ID NO: 1 or SEQ ID NO: 2 reduces the amount of PTBP1 RNA, and in certain embodiments reduces the amount of PTBP1 protein. In certain embodiments, the oligomeric agent, oligomeric compound, or antisense agent consists of a modified oligonucleotide. In certain embodiments, the oligomeric agent, oligomeric compound, or antisense agent consists of a modified oligonucleotide and a conjugate group.

In certain embodiments, contacting a cell with an oligomeric agent, an oligomeric compound, an oligomeric duplex, or an antisense agent comprising a modified oligonucleotide comprising a region that is complementary to SEQ ID NO: 1 or SEQ ID NO: 2 reduces the amount of PTBP1 RNA in the cell. In certain embodiments, contacting a cell with an oligomeric agent, an oligomeric compound, an oligomeric duplex, or an antisense agent comprising a modified oligonucleotide comprising a region that is complementary to SEQ ID NO: 1 or SEQ ID NO: 2 reduces the amount of PTBP1 protein in the cell. In certain embodiments, the cell is in vitro. In certain embodiments, the cell is in a subject. In certain embodiments, contacting a cell in a subject with an oligomeric agent, an oligomeric compound, an oligomeric duplex, or an antisense agent comprising a modified oligonucleotide comprising a region that is complementary to SEQ ID NO: 1 or SEQ ID NO: 2 ameliorates one or more symptoms or hallmarks of a neurodegenerative disease or disorder associated with PTBP1. In certain embodiments, the neurodegenerative disease or disorder associated with PTBP1 is Parkinson's disease, Huntington's disease, or Alzheimer's disease.

In certain embodiments, an oligomeric agent, an oligomeric compound, an oligomeric duplex, or an antisense agent comprising a modified oligonucleotide comprising a region that is complementary to SEQ ID NO: 1 or SEQ ID NO: 2 is capable of reducing the amount of PTBP1 RNA in vitro by at least 10%, at least 20%, at least 30%, at least 40%, at least 50%, at least 60%, at least 70%, at least 80%, or at least 90% in the standard in vitro assay. In certain embodiments, an oligomeric agent, an oligomeric compound, an oligomeric duplex, or an antisense agent comprising a modified oligonucleotide comprising a region that is complementary to SEQ ID NO: 1 or SEQ ID NO: 2 is capable of reducing the amount of PTBP1 protein in vitro by at least 10%, at least 20%, at least 30%, at least 40%, at least 50%, at least 60%, at least 70%, at least 80%, or at least 90% in the standard in vitro assay. In certain embodiments, an oligomeric agent, an oligomeric compound, an oligomeric duplex, or an antisense agent comprising a modified oligonucleotide comprising a region that is complementary to SEQ ID NO: 1 or SEQ ID NO: 2 is capable of reducing the amount of PTBP1 RNA in the cell of a subject by at least 10%, at least 20%, at least 30%, at least 40%, at least 50%, at least 60%, at least 70%, at least 80%, or at least 90%. In certain embodiments, an oligomeric agent, an oligomeric compound, an oligomeric duplex, or an antisense agent comprising a modified oligonucleotide comprising a region that is complementary to SEQ ID NO: 1 or SEQ ID NO: 2 is capable of reducing the amount of PTBP1 protein in the cell of a subject by at least 10%, at least 20%, at least 30%, at least 40%, at least 50%, at least 60%, at least 70%, at least 80%, or at least 90%.

### C. Certain Target Nucleic Acids in Certain Tissues

Reduction of PTBP1 expression in glial cells have been shown to induce differentiation into neurons (Zhou, et al., 2020, Cell 181, 590-603; Weinberg, et al., 2017, The American Society of Gene and Cell Therapy, 25, 928-934; Qian, et al. 2020, Nature, 582, 550-556; and Maimon, et al., 2021, Nature Neuroscience, 24, 1089-1099). In certain embodiments, oligomeric agents, oligomeric compounds, oligomeric duplexes, or antisense agents comprise a modified oligonucleotide comprising a region that is complementary to a target nucleic acid, wherein the target nucleic acid is expressed in a pharmacologically relevant tissue. In certain embodiments, the pharmacologically relevant tissues are the brain and spinal cord. In certain embodiments, the target nucleic acid is expressed in a pharmacologically relevant cell. In certain embodiments, the pharmacologically relevant cell is a GFAP-expressing cell. In certain embodiments, GFAP-expression cells comprise glial cells or precursor cells such as stem cell precursor cells. In certain embodiments, the pharmacologically relevant cell is a glial cell. Exemplary glial cells include, but are not limited to, astrocytes, oligodendrocytes, Muller glial cells, and radial glial cells. In certain embodiments, the pharmacologically relevant cell is an astrocyte. In certain embodiments, the pharmacologically relevant cell is a Muller glial cell. In certain embodiments, the pharmacologically relevant cell is a radial glial cell. In certain embodiments, the pharmacologically relevant cell is an oligodendrocyte. In certain embodiments, the pharmacologically relevant cell is a precursor cell, e.g., a stem cell precursor cell. In some embodiments, the pharmacologically relevant cell is a fibroblast.

### V. Certain Methods and Uses

Certain embodiments provided herein relate to methods of reducing or inhibiting PTBP1 expression or activity, which can be useful for treating, preventing, or ameliorating a neurodegenerative disease or disorder associated with PTBP1. In certain embodiments, the neurodegenerative disease or disorder associated with PTBP1 is Parkinson's disease, Huntington's disease, or Alzheimer's disease.

In certain embodiments, a method comprises administering to a subject an oligomeric agent, an oligomeric compound, a modified oligonucleotide, an oligomeric duplex, or an antisense agent, any of which having a nucleobase sequence complementary to a PTBP1 nucleic acid. In certain embodiments, the subject has or is at risk for developing a neurodegenerative disease or disorder associated with PTBP1. In certain embodiments, the subject has or is at risk for developing Parkinson's disease, Huntington's disease, or Alzheimer's disease. In certain embodiments, the subject has or is at risk for developing Parkinson's disease.

In certain embodiments, a method for treating a neurodegenerative disease or disorder associated with PTBP1 comprises administering to a subject an oligomeric agent, an oligomeric compound, a modified oligonucleotide, an oligomeric duplex, or an antisense agent, any of which having a nucleobase sequence complementary to a PTBP1 nucleic acid. In certain embodiments, the subject has or is at risk for developing a neurodegenerative disease or disorder associated with PTBP1. In certain embodiments, the subject has or is at risk for developing Parkinson's disease, Huntington's disease, or Alzheimer's disease. In certain embodiments, the subject has or is at risk for developing Parkinson's disease. In certain embodiments, at least one symptom or hallmark of the neurodegenerative disease or disorder associated with PTBP1 is ameliorated. Exemplary symptoms or hallmarks include, but are not limited to, bradykinesia, rigid muscles, tremor, impaired posture and/or balance, loss of automatic movements, impaired speech, involuntary movements, memory loss, and depression.

In certain embodiments, a method of reducing expression of PTBP1, for example RNA, or reducing the expression of PTBP1 protein in a cell comprises contacting the cell with an oligomeric agent, an oligomeric compound, a modified oligonucleotide, an oligomeric duplex, or an antisense agent, any of which having a nucleobase sequence complementary to a PTBP1 nucleic acid. In certain embodiments, the cell is a human cell. In certain embodiments, the cell is a brain cell. In certain embodiments, the cell is a GFAP-expressing cell. In certain embodiments, the cell is a glial cell or a precursor cell (e.g., a stem cell precursor cell). In certain embodiments, the cell is a glial cell (e.g., an astrocyte, an oligodendrocyte, a Muller glial cell, or a radial glial cell). In certain embodiments, the cell is an astrocyte. In certain embodiments, the cell is obtained from a subject, e.g., a subject that has or is at risk for developing a neurodegenerative disease or disorder associated with PTBP1. In certain embodiments, the subject has or is at risk for developing Parkinson's disease, Huntington's disease, or Alzheimer's disease. In certain embodiments, the subject has or is at risk for developing Parkinson's disease.

Certain embodiments are drawn to an oligomeric agent, an oligomeric compound, a modified oligonucleotide, an oligomeric duplex, or an antisense agent, any of which having a nucleobase sequence complementary to a PTBP1 nucleic acid, for use in treating a neurodegenerative disease or disorder associated with PTBP1 or for use in the manufacture of a medicament for treating a neurodegenerative disease or disorder associated with PTBP1. In certain embodiments, the neurodegenerative disease or disorder associated with PTBP1 is Parkinson's disease, Huntington's disease, or Alzheimer's disease.

In any of the methods or uses described herein, the oligomeric agent, the oligomeric compound, the modified oligonucleotide, the oligomeric duplex, or the antisense agent can be any described herein.

### VI. Certain Pharmaceutical Compositions

In certain embodiments, described herein are pharmaceutical compositions comprising one or more oligomeric agents, oligomeric compounds, oligomeric duplexes, or antisense agents. In certain embodiments, the one or more oligomeric agents, oligomeric compounds, oligomeric duplexes, or antisense agents each comprises a modified oligonucleotide. In certain embodiments, the one or more oligomeric agents, oligomeric compounds, or antisense agents each consists of a modified oligonucleotide. In certain embodiments, the pharmaceutical composition comprises a pharmaceutically acceptable diluent or carrier. In certain embodiments, a pharmaceutical composition comprises, consists essentially of, or consists of a sterile saline solution and one or more oligomeric agents, oligomeric compounds, oligomeric duplexes, or antisense agents. In certain embodiments, the sterile saline is pharmaceutical grade saline. In certain embodiments, a pharmaceutical composition comprises, consists essentially of, or consists of one or more oligomeric agents, oligomeric compounds, oligomeric duplexes, or antisense agents and sterile water. In certain embodiments, the sterile water is pharmaceutical grade water. In certain embodiments, a pharmaceutical composition comprises, consists essentially of, or consists of one or more oligomeric agents, oligomeric compounds, oligomeric duplexes, or antisense agents and phosphate-buffered saline (PBS). In certain embodiments, the sterile PBS is pharmaceutical grade PBS. In certain embodiments, a pharmaceutical composition comprises, consists essentially of, or consists of one or more oligomeric agents, oligomeric compounds, oligomeric duplexes, or antisense agents and artificial cerebrospinal fluid (“artificial CSF” or “aCSF”). In certain embodiments, the artificial cerebrospinal fluid is pharmaceutical grade artificial cerebrospinal fluid.

In certain embodiments, a pharmaceutical composition comprises an oligomeric agent, an oligomeric compound, an oligomeric duplex, an antisense agent, or a modified oligonucleotide and PBS. In certain embodiments, a pharmaceutical composition consists of an oligomeric agent, an oligomeric compound, an oligomeric duplex, an antisense agent, or a modified oligonucleotide and PBS. In certain embodiments, a pharmaceutical composition consists essentially of an oligomeric agent, an oligomeric compound, an oligomeric duplex, an antisense agent, or a modified oligonucleotide and PBS. In certain embodiments, the PBS is pharmaceutical grade.

In certain embodiments, a pharmaceutical composition comprises an oligomeric agent, an oligomeric compound, an oligomeric duplex, an antisense agent, or a modified oligonucleotide and artificial cerebrospinal fluid (aCSF). In certain embodiments, a pharmaceutical composition consists of an oligomeric agent, an oligomeric compound, an oligomeric duplex, an antisense agent, or a modified oligonucleotide and artificial cerebrospinal fluid. In certain embodiments, a pharmaceutical composition consists essentially of an oligomeric agent, an oligomeric compound, an oligomeric duplex, an antisense agent, or a modified oligonucleotide and artificial cerebrospinal fluid. In certain embodiments, the artificial cerebrospinal fluid is pharmaceutical grade.

In certain embodiments, aCSF comprises sodium chloride, potassium chloride, sodium dihydrogen phosphate dihydrate, sodium phosphate dibasic anhydrous, calcium chloride dihydrate, and magnesium chloride hexahydrate. In certain embodiments, the pH of an aCSF solution is modulated with a suitable pH-adjusting agent, for example, with acids such as hydrochloric acid and alkalis such as sodium hydroxide, to a range of from about 7.1-7.3, or to about 7.2.

In certain embodiments, pharmaceutical compositions comprise one or more oligomeric agents, oligomeric compounds, oligomeric duplexes, or antisense agents and one or more excipients. In certain embodiments, excipients are selected from water, salt solutions, alcohol, polyethylene glycols, gelatin, lactose, amylase, magnesium stearate, talc, silicic acid, viscous paraffin, hydroxymethylcellulose and polyvinylpyrrolidone.

In certain embodiments, oligomeric agents, oligomeric compounds, oligomeric duplexes, or antisense agents may be admixed with pharmaceutically acceptable active and/or inert substances for the preparation of pharmaceutical compositions or formulations. Compositions and methods for the formulation of pharmaceutical compositions depend on a number of criteria, including, but not limited to, route of administration, extent of disease, or dose to be administered.

In certain embodiments, pharmaceutical compositions comprising an oligomeric agent, an oligomeric compound, an oligomeric duplex, or an antisense agent encompass any pharmaceutically acceptable salts of the oligomeric agent, the oligomeric compound, the oligomeric duplex, or the antisense agent; esters of the oligomeric agent, the oligomeric compound, the oligomeric duplex, or the antisense agent; or salts of such esters. In certain embodiments, pharmaceutical compositions comprising oligomeric agents, oligomeric compounds, oligomeric duplexes, or antisense agents comprising one or more modified oligonucleotides, upon administration to an animal, including a human, are capable of providing (directly or indirectly) the biologically active metabolite or residue thereof. Accordingly, for example, the disclosure is also drawn to pharmaceutically acceptable salts of oligomeric agents, oligomeric compounds, oligomeric duplexes, or antisense agents, prodrugs, pharmaceutically acceptable salts of such prodrugs, and other bioequivalents. In certain embodiments, pharmaceutically acceptable salts comprise inorganic salts, such as monovalent or divalent inorganic salts. Suitable pharmaceutically acceptable salts include, but are not limited to, sodium, potassium, calcium, and magnesium salts. In certain embodiments, prodrugs comprise one or more conjugate group attached to a modified oligonucleotide, wherein the conjugate group is cleaved by endogenous nucleases within the body.

In certain embodiments, oligomeric agents, oligomeric compounds, oligomeric duplexes, or antisense agents are lyophilized and isolated as sodium salts. In certain embodiments, the sodium salt of an oligomeric agent, an oligomeric compound, an oligomeric duplex, or an antisense agent is mixed with a pharmaceutically acceptable diluent. In certain embodiments, the pharmaceutically acceptable diluent comprises sterile saline, sterile water, PBS, or aCSF. In certain embodiments, the sodium salt of an oligomeric agent, an oligomeric compound, an oligomeric duplex, or an antisense agent is mixed with PBS. In certain embodiments, the sodium salt of an oligomeric agent, an oligomeric compound, an oligomeric duplex, or an antisense agent is mixed with aCSF.

Lipid moieties have been used in nucleic acid therapies in a variety of methods. In certain such methods, the nucleic acid, such as an oligomeric agent, an oligomeric compound, an oligomeric duplex, or an antisense agent is introduced into preformed liposomes or lipoplexes made of mixtures of cationic lipids and neutral lipids. In certain methods, DNA complexes with mono- or poly-cationic lipids are formed without the presence of a neutral lipid. In certain embodiments, a lipid moiety is selected to increase distribution of a pharmaceutical agent to a particular cell or tissue. In certain embodiments, a lipid moiety is selected to increase distribution of a pharmaceutical agent to fat tissue. In certain embodiments, a lipid moiety is selected to increase distribution of a pharmaceutical agent to muscle tissue.

In certain embodiments, pharmaceutical compositions comprise a delivery system. Examples of delivery systems include, but are not limited to, liposomes and emulsions. Certain delivery systems are useful for preparing certain pharmaceutical compositions including those comprising hydrophobic compounds. In certain embodiments, certain organic solvents such as dimethylsulfoxide are used.

In certain embodiments, pharmaceutical compositions comprise one or more tissue-specific delivery molecules designed to deliver the one or more pharmaceutical agents of the present invention to specific tissues or cell types. For example, in certain embodiments, pharmaceutical compositions include liposomes coated with a tissue-specific antibody.

In certain embodiments, pharmaceutical compositions comprise a co-solvent system. Certain of such co-solvent systems comprise, for example, benzyl alcohol, a nonpolar surfactant, a water-miscible organic polymer, and an aqueous phase. In certain embodiments, such co-solvent systems are used for hydrophobic compounds. A non-limiting example of such a co-solvent system is the VPD co-solvent system, which is a solution of absolute ethanol comprising 3% w/v benzyl alcohol, 8% w/v of the nonpolar surfactant Polysorbate 80™ and 65% w/v polyethylene glycol 300. The proportions of such co-solvent systems may be varied considerably without significantly altering their solubility and toxicity characteristics. Furthermore, the identity of co-solvent components may be varied: for example, other surfactants may be used instead of Polysorbate 80™; the fraction size of polyethylene glycol may be varied; other biocompatible polymers may replace polyethylene glycol, e.g., polyvinyl pyrrolidone; and other sugars or polysaccharides may substitute for dextrose.

In certain embodiments, pharmaceutical compositions are prepared for oral administration. In certain embodiments, pharmaceutical compositions are prepared for buccal administration. In certain embodiments, a pharmaceutical composition is prepared for administration by injection (e.g., intravenous, subcutaneous, intramuscular, intrathecal (IT), intracerebroventricular (ICV), etc.). In certain of such embodiments, a pharmaceutical composition comprises a carrier and is formulated in aqueous solution, such as water or physiologically compatible buffers such as Hanks's solution, Ringer's solution, or physiological saline buffer. In certain embodiments, other ingredients are included (e.g., ingredients that aid in solubility or serve as preservatives). In certain embodiments, injectable suspensions are prepared using appropriate liquid carriers, suspending agents and the like. Certain pharmaceutical compositions for injection are presented in unit dosage form, e.g., in ampoules or in multi-dose containers. Certain pharmaceutical compositions for injection are suspensions, solutions or emulsions in oily or aqueous vehicles, and may contain formulatory agents such as suspending, stabilizing and/or dispersing agents. Certain solvents suitable for use in pharmaceutical compositions for injection include, but are not limited to, lipophilic solvents and fatty oils, such as sesame oil, synthetic fatty acid esters, such as ethyl oleate or triglycerides, and liposomes.

Under certain conditions, certain compounds disclosed herein act as acids. Although such compounds may be drawn or described in protonated (free acid) form, or ionized and in association with a cation (salt) form, aqueous solutions of such compounds exist in equilibrium among such forms. For example, a phosphate linkage of an oligonucleotide in aqueous solution exists in equilibrium among free acid, anion, and salt forms. Unless otherwise indicated, compounds described herein are intended to include all such forms. Moreover, certain oligonucleotides have several such linkages, each of which is in equilibrium. Thus, oligonucleotides in solution exist in an ensemble of forms at multiple positions all at equilibrium. The term “oligonucleotide” is intended to include all such forms. Drawn structures necessarily depict a single form. Nevertheless, unless otherwise indicated, such drawings are likewise intended to include corresponding forms. Herein, a structure depicting the free acid of a compound followed by the term “or a pharmaceutically acceptable salt thereof” expressly includes all such forms that may be fully or partially protonated/de-protonated/in association with a cation. In certain embodiments, one or more specific cation is identified. The cations may include, but are not limited to, sodium, potassium, calcium, and magnesium.

In certain embodiments, modified oligonucleotides, oligomeric agents, oligomeric compounds, oligomeric duplexes, or antisense agents are in aqueous solution with sodium. In certain embodiments, modified oligonucleotides, oligomeric agents, oligomeric compounds, oligomeric duplexes, or antisense agents are in aqueous solution with potassium. In certain embodiments, modified oligonucleotides, oligomeric agents, oligomeric compounds, oligomeric duplexes, or antisense agents are in aqueous solution with calcium. In certain embodiments, modified oligonucleotides, oligomeric agents, oligomeric compounds, oligomeric duplexes, or antisense agents are in aqueous solution with magnesium. In certain embodiments, modified oligonucleotides, oligomeric agents, oligomeric compounds, oligomeric duplexes, or antisense agents are in PBS. In certain embodiments, modified oligonucleotides, oligomeric agents, oligomeric compounds, oligomeric duplexes, or antisense agents are in aCSF. In certain embodiments, modified oligonucleotides, oligomeric agents, oligomeric compounds, oligomeric duplexes, or antisense agents are in water. In certain such embodiments, the pH of the solution is adjusted with NaOH and/or HCl to achieve a desired pH.

### VII. Certain Hotspot Regions

### 1. Nucleobases 16,938-16,960 of SEQ ID NO: 1

In certain embodiments, nucleobases of SEQ ID NO: 1 comprise a hotspot region. In certain embodiments, modified oligonucleotides are complementary to the nucleobase sequence of an equal length portion within nucleobases 16,938-16,960 of SEQ ID NO: 1. In certain embodiments, modified oligonucleotides are 20 nucleobases in length. In certain embodiments, modified oligonucleotides are gapmers. In certain embodiments, the gapmers are MOE gapmers. In certain embodiments, the sugar motif for the gapmers is 5′-eeeeeddddddddddeeeee-3′, wherein each “d” represents a 2′-β-D-deoxyribosyl sugar moiety, and each “e” represents a 2′-MOE sugar moiety. In certain embodiments, the nucleosides of the modified oligonucleotides are linked by a combination of phosphodiester and phosphorothioate internucleoside linkages. In certain embodiments, the nucleosides of the modified oligonucleotides are linked by phosphorothioate internucleoside linkages. In certain embodiments, the internucleoside linkage motif for the gapmers is sooosssssssssssooss; wherein each “o” represents a phosphodiester internucleoside linkage, and each “s” represents a phosphorothioate internucleoside linkage.

The nucleobase sequences of SEQ ID NOs: 46, 130, and 464 are complementary to the nucleobase sequence of an equal length portion within nucleobases 16,938-16,960 of SEQ ID NO: 1.

The nucleobase sequences of Compound Nos: 1587654, 1587661, and 1587861 are complementary to the nucleobase sequence of an equal length portion within nucleobases 16,938-16,960 of SEQ ID NO: 1.

In certain embodiments, modified oligonucleotides complementary to the nucleobase sequence of an equal length portion within nucleobases 16,938-16,960 of SEQ ID NO: 1 achieve at least 49% reduction of PTBP1 mRNA in the standard in vitro cell assay. In certain embodiments, modified oligonucleotides complementary to the nucleobase sequence of an equal length portion within nucleobases 16,938-16,960 of SEQ ID NO: 1 achieve an average of 62% reduction of PTBP1 mRNA in the standard in vitro cell assay.

### 2. Nucleobases 17,538-17,573 of SEQ ID NO: 1

In certain embodiments, nucleobases of SEQ ID NO: 1 comprise a hotspot region. In certain embodiments, modified oligonucleotides are complementary to the nucleobase sequence of an equal length portion within nucleobases 17,538-17,573 of SEQ ID NO: 1. In certain embodiments, modified oligonucleotides are 20 nucleobases in length. In certain embodiments, modified oligonucleotides are gapmers. In certain embodiments, the gapmers are MOE gapmers. In certain embodiments, the sugar motif for the gapmers is 5′-eeeeeddddddddddeeeee-3′, wherein each “d” represents a 2′-β-D-deoxyribosyl sugar moiety, and each “e” represents a 2′-MOE sugar moiety. In certain embodiments, the nucleosides of the modified oligonucleotides are linked by a combination of phosphodiester and phosphorothioate internucleoside linkages. In certain embodiments, the nucleosides of the modified oligonucleotides are linked by phosphorothioate internucleoside linkages. In certain embodiments, the internucleoside linkage motif for the gapmers is sooosssssssssssooss; wherein each “o” represents a phosphodiester internucleoside linkage, and each “s” represents a phosphorothioate internucleoside linkage.

The nucleobase sequences of SEQ ID NOs: 84, 145, 170, 239, 318, 339, and 408 are complementary to the nucleobase sequence of an equal length portion within nucleobases 17,538-17,573 of SEQ ID NO: 1.

The nucleobase sequences of Compound Nos: 1587510, 1587567, 1587745, 1587860, 1587886, 1587897, and 1587921 are complementary to the nucleobase sequence of an equal length portion within nucleobases 17,538-17,573 of SEQ ID NO: 1.

In certain embodiments, modified oligonucleotides complementary to the nucleobase sequence of an equal length portion within nucleobases 17,538-17,573 of SEQ ID NO: 1 achieve at least 34% reduction of PTBP1 mRNA in the standard in vitro cell assay. In certain embodiments, modified oligonucleotides complementary to the nucleobase sequence of an equal length portion within nucleobases 17,538-17,573 of SEQ ID NO: 1 achieve an average of 58% reduction of PTBP1 mRNA in the standard in vitro cell assay.

### 3. Nucleobases 17,988-18,016 of SEQ ID NO: 1

In certain embodiments, nucleobases of SEQ ID NO: 1 comprise a hotspot region. In certain embodiments, modified oligonucleotides are complementary to the nucleobase sequence of an equal length portion within nucleobases 17,988-18,016 of SEQ ID NO: 1. In certain embodiments, modified oligonucleotides are 20 nucleobases in length. In certain embodiments, modified oligonucleotides are gapmers. In certain embodiments, the gapmers are MOE gapmers. In certain embodiments, the sugar motif for the gapmers is 5′-eeeeeddddddddddeeeee-3′, wherein each “d” represents a 2′-β-D-deoxyribosyl sugar moiety, and each “e” represents a 2′-MOE sugar moiety. In certain embodiments, the nucleosides of the modified oligonucleotides are linked by a combination of phosphodiester and phosphorothioate internucleoside linkages. In certain embodiments, the nucleosides of the modified oligonucleotides are linked by phosphorothioate internucleoside linkages. In certain embodiments, the internucleoside linkage motif for the gapmers is sooosssssssssssooss; wherein each “o” represents a phosphodiester internucleoside linkage, and each “s” represents a phosphorothioate internucleoside linkage.

The nucleobase sequences of SEQ ID NOs: 21, 253, 330, and 407 are complementary to the nucleobase sequence of an equal length portion within nucleobases 17,988-18,016 of SEQ ID NO: 1.

The nucleobase sequences of Compound Nos: 1587321, 1587329, 1587502, and 1587506 are complementary to the nucleobase sequence of an equal length portion within nucleobases 17,988-18,016 of SEQ ID NO: 1.

In certain embodiments, modified oligonucleotides complementary to the nucleobase sequence of an equal length portion within nucleobases 17,988-18,016 of SEQ ID NO: 1 achieve at least 19% reduction of PTBP1 mRNA in the standard in vitro cell assay. In certain embodiments, modified oligonucleotides complementary to the nucleobase sequence of an equal length portion within nucleobases 17,988-18,016 of SEQ ID NO: 1 achieve an average of 49% reduction of PTBP1 mRNA in the standard in vitro cell assay.

### NONLIMITING DISCLOSURE AND INCORPORATION BY REFERENCE

Each of the literature and patent publications listed herein is incorporated by reference in its entirety.

While certain compounds and compositions described herein have been described with specificity in accordance with certain embodiments, the following examples serve only to illustrate the compounds described herein and are not intended to limit the same. Each of the references, GenBank accession numbers, ENSEMBL identifiers, and the like recited in the present application is incorporated herein by reference in its entirety.

Although the sequence listing accompanying this filing identifies each sequence as either “RNA” or “DNA” as required, in reality, those sequences may be modified with any combination of chemical modifications. One of skill in the art will readily appreciate that such designation as “RNA” or “DNA” to describe modified oligonucleotides is, in certain instances, arbitrary. For example, an oligonucleotide comprising a nucleoside comprising a 2′-OH sugar moiety and a thymine base could be described as a DNA having a modified sugar (2′-OH in place of one 2′-H of DNA) or as an RNA having a modified base (thymine (methylated uracil) in place of an uracil of RNA). Accordingly, nucleic acid sequences provided herein, including, but not limited to those in the sequence listing, are intended to encompass nucleic acids containing any combination of natural or modified RNA and/or DNA, including, but not limited to such nucleic acids having modified nucleobases. By way of further example and without limitation, an oligomeric compound having the nucleobase sequence “ATCGATCG” encompasses any oligomeric compounds having such nucleobase sequence, whether modified or unmodified, including, but not limited to, such compounds comprising RNA bases, such as those having sequence “AUCGAUCG” and those having some DNA bases and some RNA bases such as “AUCGATCG” and oligomeric compounds having other modified nucleobases, such as “AT^{m}CGAUCG,” wherein ^{m}C indicates a cytosine base comprising a methyl group at the 5-position.

Certain compounds described herein (e.g., modified oligonucleotides) have one or more asymmetric center and thus give rise to enantiomers, diastereomers, and other stereoisomeric configurations that may be defined, in terms of absolute stereochemistry, as (R) or (S), as α or β such as for sugar anomers, or as (D) or (L), such as for amino acids, etc. Compounds provided herein that are drawn or described as having certain stereoisomeric configurations include only the indicated compounds. Compounds provided herein that are drawn or described with undefined stereochemistry include all such possible isomers, including their stereorandom and optically pure forms, unless specified otherwise. Likewise, tautomeric forms of the compounds herein are also included unless otherwise indicated. Unless otherwise indicated, compounds described herein are intended to include corresponding salt forms.

The compounds described herein include variations in which one or more atoms are replaced with a non-radioactive isotope or radioactive isotope of the indicated element. For example, compounds herein that comprise hydrogen atoms encompass all possible deuterium substitutions for each of the ¹H hydrogen atoms. Isotopic substitutions encompassed by the compounds herein include but are not limited to: ²H or ³H in place of ¹H, ¹³C or ¹⁴C in place of ¹²C, ¹⁵N in place of ¹⁴N, ¹⁷O or ¹⁸O in place of ¹⁶O, and ³³S, ³⁴S, ³⁵S, or ³⁶S in place of ³²S. In certain embodiments, non-radioactive isotopic substitutions may impart new properties on the oligomeric compound that are beneficial for use as a therapeutic or research tool. In certain embodiments, radioactive isotopic substitutions may make the compound suitable for research or diagnostic purposes such as imaging.

### EXAMPLES

The following examples illustrate certain embodiments of the present disclosure and are not limiting. Moreover, where specific embodiments are provided, the inventors have contemplated generic application of those specific embodiments. For example, disclosure of an oligonucleotide having a particular motif provides reasonable support for additional oligonucleotides having the same or similar motif. And, for example, where a particular high-affinity modification appears at a particular position, other high-affinity modifications at the same position are considered suitable, unless otherwise indicated.

### Example 1: Effect of 5-10-5 MOE Modified Oligonucleotides with Mixed PO/PS Linkages on Human PTBP1 RNA In Vitro, Single Dose

Modified oligonucleotides complementary to a human PTBP1 nucleic acid were designed and tested for their single dose effects on PTBP1 RNA in vitro. The modified oligonucleotides were tested in a series of experiments that had the same culture conditions.

The modified oligonucleotides in the table below are 5-10-5 MOE modified oligonucleotides with mixed PO/PS internucleoside linkages. The modified oligonucleotides are 20 nucleosides in length, wherein the central gap segment consists of ten 2′-β-D-deoxynucleosides, and wherein the 5′ and 3′ wings each consist of five 2′-MOE modified nucleosides. The sugar motif of the modified oligonucleotides is (from 5′ to 3′): eeeeeddddddddddeeeee; wherein each “d” represents a 2′-β-D-deoxyribosyl sugar moiety, and each “e” represents a 2′-MOE sugar moiety. The internucleoside linkage motif of the modified oligonucleotides is (from 5′ to 3′): sooosssssssssssooss; wherein each “o” represents a phosphodiester internucleoside linkage, and each “s” represents a phosphorothioate internucleoside linkage. Each cytosine residue is a 5-methylcytosine.

“Start site” indicates the 5′-most nucleoside to which the modified oligonucleotide is complementary in the target nucleic acid sequence. “Stop site” indicates the 3′-most nucleoside to which the modified oligonucleotide is complementary in the target nucleic acid sequence. Each modified oligonucleotide listed in the table below is 100% complementary to either SEQ ID NO: 1 (GENBANK Accession No. NC_000019.10, truncated from nucleosides 794001 to 815000), or to SEQ ID NO: 2 (GENBANK Accession No. NM_002819.4), or to both. “N/A” indicates that the modified oligonucleotide is not 100% complementary to that particular target nucleic acid sequence.

Cultured A431 cells were treated with modified oligonucleotide at a concentration of 3,000 nM by free uptake at a density of 11,000 cells per well. After a treatment period of approximately 48 hours, total RNA was isolated from the cells, and PTBP1 RNA levels were measured by quantitative real-time RTPCR. PTBP1 RNA levels were measured by human primer-probe set RTS54047 (forward sequence AGGAAATTCTGTATTGCTGGTCA, designated herein as SEQ ID NO: 9; reverse sequence CCTTCTTATTGAACAGGATCTTCAC, designated herein as SEQ ID NO: 10; probe sequence AGTCACACCCCAAAGCCTCTTTATTCTTT, designated herein as SEQ ID NO: 11). PTBP1 RNA levels were noralized to total RNA content, as measured by RIBOGREEN®. Reduction of PTBP1 RNA is presented in the table below as percent PTBP1 RNA relative to the amount in untreated control cells (% UTC). The values marked with a “T” indicate that the modified oligonucleotide is complementary to the amplicon region of the primer-probe set. Additional assays may be used to measure the potency and efficacy of the modified oligonucleotides complementary to the amplicon region. Each separate experiment described in this example is identified by an Assay Identification letter in the table column labeled “AID”.

TABLE 1
Reduction of PTBP1 RNA by 5-10-5 MOE modified oligonucleotides
with mixed PO/PS linkages at a concentration of 3000 nM in
A431 cells

| | SEQ ID | SEQ ID | SEQ ID | SEQ ID | | | | |
|---|---|---|---|---|---|---|---|---|
| | No: 1 | No: 1 | No: 2 | No: 2 | | PTBP1 | | SEQ |
| Compound | Start | Stop | Start | Stop | Sequence | (% | | ID |
| Number | Site | Site | Site | Site | (5′ to 3′) | UTC) | AID | NO |
| 1587321 | 17988 | 18007 | 2942 | 2961 | GTTACACGCTTTTACGGAAA | 81 | A | 21 |
| 1587509 | 6298 | 6317 | N/A | N/A | ACATTCATTTTTCTCTTGCC | 74 | A | 22 |
| 1587511 | 16301 | 16320 | N/A | N/A | CCACTTTTTTTTTTCCAGAT | 61 | A | 23 |
| 1587515 | 5811 | 5830 | N/A | N/A | TGGCTCTTTTCCTATCAAAA | 87 | A | 24 |
| 1587522 | 16553 | 16572 | 1580 | 1599 | ACCTTGAGATCCTCCTCGGA | 118 | A | 25 |
| 1587523 | 6288 | 6307 | N/A | N/A | TTCTCTTGCCCCAAACACCA | 108 | A | 26 |
| 1587526 | 8462 | 8481 | N/A | N/A | TGGATATCACACCCTGTCCC | 107 | A | 27 |
| 1587535 | 17484 | 17503 | 2438 | 2457 | AAAGATTAGAATATTTGGTC | 127 | A | 28 |
| 1587543 | 11880 | 11899 | N/A | N/A | GCCACTCAAATTCAACTACT | 110 | A | 29 |
| 1587546 | 7692 | 7711 | N/A | N/A | TCCAACCTTCGCCTCCCACA | 115 | A | 30 |
| 1587557 | 9335 | 9354 | N/A | N/A | TGCTCCTTTTTCCAAATTCT | 81 | A | 31 |
| 1587560 | 8886 | 8905 | N/A | N/A | GCGGCTCCATCACTTCTGAC | 94 | A | 32 |
| 1587568 | 5747 | 5766 | N/A | N/A | CGGCACGATTCCAGCCTCTA | 97 | A | 33 |
| 1587575 | 8872 | 8891 | N/A | N/A | TCTGACCTGTCCATTTTCTC | 118 | A | 34 |
| 1587576 | 9341 | 9360 | N/A | N/A | AAACACTGCTCCTTTTTCCA | 88 | A | 35 |
| 1587577 | 17562 | 17581 | 2516 | 2535 | TTTAGATTACACTCATACCA | 90 | A | 36 |
| 1587580 | 17522 | 17541 | 2476 | 2495 | AAGTTACTTAAAACTATTTC | 96 | A | 37 |
| 1587585 | 15990 | 16009 | N/A | N/A | GATAACATTTCACAAGTAAC | 86 | A | 38 |
| 1587590 | 4845 | 4864 | N/A | N/A | CCTCAGTTACTGATCAACCC | 106 | A | 39 |
| 1587599 | 17210 | 17229 | 2164 | 2183 | ACCCCCCCACCCCTTGGGTC | 71 | A | 40 |
| 1587608 | 18003 | 18022 | 2957 | 2976 | AATATTTACACCCTTGTTAC | 102 | A | 41 |
| 1587616 | 14988 | 15007 | N/A | N/A | ATGTGTGCATTATACATTCT | 110 | A | 42 |
| 1587626 | 10844 | 10863 | 728 | 747 | ATGATCTTCAACACTGTGCC | 120 | A | 43 |
| 1587629 | 18011 | 18030 | 2965 | 2984 | AAATTATAAATATTTACACC | 116 | A | 44 |
| 1587630 | 10131 | 10150 | 317 | 336 | GAGATGACTTCCCCCTCCGT | 87 | A | 45 |
| 1587654 | 16940 | 16959 | 1894 | 1913 | CAGGTAAACTGATTTCTCTT | 51 | A | 46 |
| 1587658 | 8851 | 8870 | N/A | N/A | CCAGAAACAATCTATTCACT | 130 | A | 47 |
| 1587660 | 18089 | 18108 | 3043 | 3062 | TTGCTGTTAGCAAAATATAC | 95 | A | 48 |
| 1587671 | 17858 | 17877 | 2812 | 2831 | ACAGAAATGAGACTTTGGTC | 89 | A | 49 |
| 1587673 | 15730 | 15749 | N/A | N/A | GCTAATTTTAACCAATTGTT | 121 | A | 50 |
| 1587674 | 6705 | 6724 | N/A | N/A | TACCTCTCCCTCCTCCCACA | 108 | A | 51 |
| 1587678 | 4388 | 4407 | N/A | N/A | ATGCGAAACATCTCCAGCCT | 142 | A | 52 |
| 1587708 | 14379 | 14398 | 1279 | 1298 | TGAACAGGATCTTCACGCGC | 21† | A | 53 |
| 1587709 | 12522 | 12541 | 1191 | 1210 | GCTGACCAGCAATACAGAAT | 13† | A | 54 |
| 1587713 | 8866 | 8885 | N/A | N/A | CTGTCCATTTTCTCTCCAGA | 96 | A | 55 |
| 1587714 | 10306 | 10325 | 409 | 428 | TGGCAGCCTCCTCCGTGTTC | 117 | A | 56 |
| 1587715 | 12511 | 12530 | 1180 | 1199 | ATACAGAATTTCCTGCCCCC | 74† | A | 57 |
| 1587718 | 8815 | 8834 | N/A | N/A | TTGGATCAACTTCTAAGAAT | 104 | A | 58 |
| 1587720 | 15915 | 15934 | N/A | N/A | TGCATTTCCAAATACTTGCA | 105 | A | 59 |
| 1587723 | 14934 | 14953 | N/A | N/A | GTGTCCAACGACCCCCTCCC | 96 | A | 60 |
| 1587725 | 5838 | 5857 | N/A | N/A | TCTATCCAGGTCCCCTCCAC | 84 | A | 61 |
| 1587727 | 12990 | 13009 | N/A | N/A | GTTTCACCCAAGCTCTCCCC | 79† | A | 62 |
| 1587729 | 16387 | 16406 | N/A | N/A | TATCAAATCATGTCCACAAT | 41 | A | 63 |
| 1587730 | 14633 | 14652 | 1440 | 1459 | GCCCTGGTCCTCCTGGCCCT | 112 | A | 64 |
| 1587732 | 7107 | 7126 | N/A | N/A | GGTGTCTTTAACCCCACACT | 111 | A | 65 |
| 1587733 | 17623 | 17642 | 2577 | 2596 | GTGACTTAATTACAAGGAAT | 62 | A | 66 |
| 1587742 | 17515 | 17534 | 2469 | 2488 | TTAAAACTATTTCTTTTGCA | 91 | A | 67 |
| 1587756 | 13457 | 13476 | N/A | N/A | CTCCACACACATTCCAGTTA | 104† | A | 68 |
| 1587758 | 8557 | 8576 | N/A | N/A | AACAAAGGAGCCTCCATCTC | 120 | A | 69 |
| 1587759 | 6330 | 6349 | N/A | N/A | GCTGCAAGATTCGCACACCC | 98 | A | 70 |
| 1587765 | 17725 | 17744 | 2679 | 2698 | CGCCCCTTCCCTGAAGCTCA | 101 | A | 71 |
| 1587779 | 5569 | 5588 | N/A | N/A | CCAACTCCAGATTCCAGACC | 110 | A | 72 |
| 1587785 | 16698 | 16717 | 1652 | 1671 | ATCAGTGCCATCTTGCGGTC | 97 | A | 73 |
| 1587787 | 13468 | 13487 | N/A | N/A | TATCAGTCACACTCCACACA | 95† | A | 74 |
| 1587790 | 17019 | 17038 | 1973 | 1992 | TGCCACAGTCACCAAGAGCC | 106 | A | 75 |
| 1587797 | 18069 | 18088 | 3023 | 3042 | ATTTGTGTCACCATAAAAAA | 101 | A | 76 |
| 1587806 | 9051 | 9070 | N/A | N/A | CTGCCTCCCTCACTCTCGCA | 105 | A | 77 |
| 1587814 | 7441 | 7460 | N/A | N/A | CCTGAAGCCCTAATCTCATT | 69 | A | 78 |
| 1587822 | 6401 | 6420 | N/A | N/A | AGGACCTTCTTCTTCTCCAG | 108 | A | 79 |
| 1587851 | 16310 | 16329 | N/A | N/A | CACGCTCCTCCACTTTTTTT | 114 | A | 80 |
| 1587864 | 18234 | 18253 | 3188 | 3207 | GTCCACAGCGAACACAGGGC | 109 | A | 81 |
| 1587878 | 18287 | 18306 | 3241 | 3260 | ACAGAAGATTTATTCGAAGT | 116 | A | 82 |
| 1587879 | 17555 | 17574 | 2509 | 2528 | TACACTCATACCATTGTATC | 108 | A | 83 |
| 1587886 | 17545 | 17564 | 2499 | 2518 | CCATTGTATCATCTTGCTAT | 39 | A | 84 |
| 1587888 | 6461 | 6480 | N/A | N/A | ATCATTAACCAACTACCCCA | 103 | A | 85 |
| 1587893 | 16885 | 16904 | 1839 | 1858 | TCAGCTGTTTTTAAAGTGGC | 83 | A | 86 |
| 1587898 | 17429 | 17448 | 2383 | 2402 | TGCAAAGTATATAATACAGA | 126 | A | 87 |
| 1587907 | 9430 | 9449 | N/A | N/A | CCATACCCACACCCACACTT | 92 | A | 88 |
| 1587912 | 15803 | 15822 | N/A | N/A | CGCGAAGTGAACCTCCCACC | 107 | A | 89 |
| 1587922 | 11459 | 11478 | N/A | N/A | AGACACTAATCATCGCACCC | 64 | A | 90 |
| 1587943 | 6127 | 6146 | N/A | N/A | AACAATGTTCGAAACCCCAT | 32 | A | 91 |
| 1587944 | 8449 | 8468 | N/A | N/A | CTGTCCCCCCTCAGACAACC | 108 | A | 92 |
| 1587950 | 8323 | 8342 | N/A | N/A | AGGCCGCCTCCCCTCCACCC | 91 | A | 93 |
| 1587955 | 6382 | 6401 | N/A | N/A | GGTTCTGACCCCCTTTCCTC | 86 | A | 94 |
| 1587956 | 13941 | 13960 | N/A | N/A | CATCAAAATCATCTCAATTA | 113 | A | 95 |
| 1587964 | 17231 | 17250 | 2185 | 2204 | CCCCTGCCTCTCTCTGGTGT | 111 | A | 96 |
| 1587965 | 9602 | 9621 | 187 | 206 | TGCTGCTCATGATAAACGGT | 114 | A | 97 |
| 1587967 | 10047 | 10066 | 233 | 252 | CCTTTGAACTTCTTGCTGTC | 107 | A | 98 |
| 1587321 | 17988 | 18007 | 2942 | 2961 | GTTACACGCTTTTACGGAAA | 47 | B | 21 |
| 1587338 | 17804 | 17823 | 2758 | 2777 | AGAGCAAGTTTTCTAGGCGA | 33 | B | 99 |
| 1587501 | 16873 | 16892 | 1827 | 1846 | AAAGTGGCTTTTCTCTGGAA | 69 | B | 100 |
| 1587508 | 6297 | 6316 | N/A | N/A | CATTCATTTTTCTCTTGCCC | 57 | B | 101 |
| 1587514 | 8865 | 8884 | N/A | N/A | TGTCCATTTTCTCTCCAGAA | 84 | B | 102 |
| 1587538 | 16365 | 16384 | N/A | N/A | TCTGAAGCAGCTTCTAACGA | 73 | B | 103 |
| 1587540 | 16309 | 16328 | N/A | N/A | ACGCTCCTCCACTTTTTTTT | 77 | B | 104 |
| 1587555 | 17561 | 17580 | 2515 | 2534 | TTAGATTACACTCATACCAT | 101 | B | 105 |
| 1587556 | 9027 | 9046 | N/A | N/A | CCCCTCCTCTCCTGTTTTCC | 60 | B | 106 |
| 1587558 | 15676 | 15695 | V/A | N/A | GCTGGTCTTAACTCTTGGGT | 75 | B | 107 |
| 1587562 | 18087 | 18106 | 3041 | 3060 | GCTGTTAGCAAAATATACAT | 71 | B | 108 |
| 1587570 | 14419 | 14438 | 1319 | 1338 | TTGCCGTCCGCCATCTGCAC | 87 | B | 109 |
| 1587578 | 4387 | 4406 | N/A | N/A | TGCGAAACATCTCCAGCCTC | 66 | B | 110 |
| 1587581 | 7405 | 7424 | N/A | N/A | CTGGCATTGCCCACAACTGT | 79 | B | 111 |
| 1587595 | 16551 | 16570 | 1578 | 1597 | CTTGAGATCCTCCTCGGAGA | 62 | B | 112 |
| 1587597 | 8885 | 8904 | N/A | N/A | CGGCTCCATCACTTCTGACC | 59 | B | 113 |
| 1587601 | 9340 | 9359 | N/A | N/A | AACACTGCTCCTTTTTCCAA | 88 | B | 114 |
| 1587605 | 7663 | 7682 | N/A | N/A | TTCCTGTCTGCTCTCCCCTC | 85 | B | 115 |
| 1587609 | 17105 | 17124 | 2059 | 2078 | CCTGCAGGCCCCACGCCCGA | 58 | B | 116 |
| 1587612 | 17420 | 17439 | 2374 | 2393 | TATAATACAGAGATTATGTC | 78 | B | 117 |
| 1587614 | 18010 | 18029 | 2964 | 2983 | AATTATAAATATTTACACCC | 77 | B | 118 |
| 1587621 | 8300 | 8319 | N/A | N/A | TTCAGCTCCACTCCTGCCCT | 74 | B | 119 |
| 1587622 | 12509 | 12528 | 1178 | 1197 | ACAGAATTTCCTGCCCCCGC | 54† | B | 120 |
| 1587623 | 11452 | 11471 | N/A | N/A | AATCATCGCACCCACAACCT | 117 | B | 121 |
| 1587627 | 13859 | 13878 | N/A | N/A | GTGTGACTCTCTTTAGAGAC | 63† | B | 122 |
| 1587637 | 8461 | 8480 | N/A | N/A | GGATATCACACCCTGTCCCC | 72 | B | 123 |
| 1587639 | 16005 | 16024 | N/A | N/A | CCTTGTTGATTTTCTGATAA | 68 | B | 124 |
| 1587642 | 5418 | 5437 | 120 | 139 | ACCAACGGCTATATCTGGGA | 76 | B | 125 |
| 1587645 | 5698 | 5717 | N/A | N/A | GCACACAGAGCCCTCGCTGC | 76 | B | 126 |
| 1587646 | 18002 | 18021 | 2956 | 2975 | ATATTTACACCCTTGTTACA | 68 | B | 127 |
| 1587652 | 6120 | 6139 | N/A | N/A | TTCGAAACCCCATCTTTACC | 87 | B | 128 |
| 1587657 | 6329 | 6348 | N/A | N/A | CTGCAAGATTCGCACACCCC | 69 | B | 129 |
| 1587661 | 16938 | 16957 | 1892 | 1911 | GGTAAACTGATTTCTCTTTA | 21 | B | 130 |
| 1587662 | 14987 | 15006 | N/A | N/A | TGTGTGCATTATACATTCTC | 73 | B | 131 |
| 1587679 | 6399 | 6418 | N/A | N/A | GACCTTCTTCTTCTCCAGGT | 78 | B | 132 |
| 1587683 | 9426 | 9445 | N/A | N/A | ACCCACACCCACACTTCCAC | 93 | B | 133 |
| 1587684 | 18205 | 18224 | 3159 | 3178 | GAGGAGTTTCTCAAACGGTT | 78 | B | 134 |
| 1587691 | 13455 | 13474 | N/A | N/A | CCACACACATTCCAGTTACA | 86† | B | 135 |
| 1587710 | 17521 | 17540 | 2475 | 2494 | AGTTACTTAAAACTATTTCT | 96 | B | 136 |
| 1587717 | 10680 | 10699 | 690 | 709 | GTGCAGCACATCCAGGGTCA | 106 | B | 137 |
| 1587721 | 8794 | 8813 | N/A | N/A | GCACTAGACTGATCCTAGGC | 74 | B | 138 |
| 1587724 | 17514 | 17533 | 2468 | 2487 | TAAAACTATTTCTTTTGCAT | 37 | B | 139 |
| 1587726 | 9600 | 9619 | 185 | 204 | CTGCTCATGATAAACGGTCC | 77 | B | 140 |
| 1587737 | 10045 | 10064 | 231 | 250 | TTTGAACTTCTTGCTGTCAT | 109 | B | 141 |
| 1587741 | 18286 | 18305 | 3240 | 3259 | CAGAAGATTTATTCGAAGTC | 82 | B | 142 |
| 1587743 | 8847 | 8866 | N/A | N/A | AAACAATCTATTCACTGGTT | 97 | B | 143 |
| 1587744 | 17657 | 17676 | 2611 | 2630 | GCCCCGCCTGCTCTCTGGAA | 72 | B | 144 |
| 1587745 | 17538 | 17557 | 2492 | 2511 | ATCATCTTGCTATAAAAAGT | 66 | B | 145 |
| 1587754 | 12988 | 13007 | N/A | N/A | TTCACCCAAGCTCTCCCCGA | 71† | B | 146 |
| 1587755 | 5835 | 5854 | N/A | N/A | ATCCAGGTCCCCTCCACAGC | 89 | B | 147 |
| 1587762 | 10160 | 10179 | 346 | 365 | TGGTGACCTTCCCAAAGGGC | 81 | B | 148 |
| 1587764 | 12521 | 12540 | 1190 | 1209 | CTGACCAGCAATACAGAATT | 34† | B | 149 |
| 1587782 | 11877 | 11896 | N/A | N/A | ACTCAAATTCAACTACTAGA | 114 | B | 150 |
| 1587786 | 16688 | 16707 | N/A | N/A | TCTTGCGGTCCTTCCTGCAA | 73 | B | 151 |
| 1587796 | 14932 | 14951 | N/A | N/A | GTCCAACGACCCCCTCCCTT | 83 | B | 152 |
| 1587798 | 17018 | 17037 | 1972 | 1991 | GCCACAGTCACCAAGAGCCT | 111 | B | 153 |
| 1587801 | 6678 | 6697 | N/A | N/A | AACAGGTTTTATCTCCAGGA | 49 | B | 154 |
| 1587821 | 6275 | 6294 | N/A | N/A | AACACCACCTTTGACAGTTT | 64 | B | 155 |
| 1587828 | 5810 | 5829 | N/A | N/A | GGCTCTTTTCCTATCAAAAC | 92 | B | 156 |
| 1587837 | 6409 | 6428 | N/A | N/A | GCCTCCCCAGGACCTTCTTC | 91 | B | 157 |
| 1587839 | 10129 | 10148 | 315 | 334 | GATGACTTCCCCCTCCGTGA | 59 | B | 158 |
| 1587842 | 6381 | 6400 | N/A | N/A | GTTCTGACCCCCTTTCCTCA | 84 | B | 159 |
| 1587843 | 17483 | 17502 | 2437 | 2456 | AAGATTAGAATATTTGGTCA | 61 | B | 160 |
| 1587846 | 4816 | 4835 | N/A | N/A | CCGACCCGACTCTCCCTCCT | 103 | B | 161 |
| 1587862 | 15922 | 15941 | N/A | N/A | ATGTACCTGCATTTCCAAAT | 33 | B | 162 |
| 1587872 | 9331 | 9350 | N/A | N/A | CCTTTTTCCAAATTCTGGAA | 110 | B | 163 |
| 1587880 | 6754 | 6773 | N/A | N/A | CTGCAAGCCAGACACATCTA | 85 | B | 164 |
| 1587902 | 15914 | 15933 | N/A | N/A | GCATTTCCAAATACTTGCAT | 59 | B | 165 |
| 1587910 | 8435 | 8454 | N/A | N/A | ACAACCCCCAGCTCCACACA | 84 | B | 166 |
| 1587911 | 14378 | 14397 | 1278 | 1297 | GAACAGGATCTTCACGCGCT | 9† | B | 167 |
| 1587913 | 17595 | 17614 | 2549 | 2568 | AGTAACAGCATACAAGGTAA | 54 | B | 168 |
| 1587914 | 15802 | 15821 | N/A | N/A | GCGAAGTGAACCTCCCACCT | 72 | B | 169 |
| 1587921 | 17554 | 17573 | 2508 | 2527 | ACACTCATACCATTGTATCA | 51 | B | 170 |
| 1587927 | 13467 | 13486 | N/A | N/A | ATCAGTCACACTCCACACAC | 79† | B | 171 |
| 1587929 | 18065 | 18084 | 3019 | 3038 | GTGTCACCATAAAAAACCGC | 21 | B | 172 |
| 1587932 | 8552 | 8571 | N/A | N/A | AGGAGCCTCCATCTCCCGTA | 100 | B | 173 |
| 1587946 | 8871 | 8890 | N/A | N/A | CTGACCTGTCCATTTTCTCT | 108 | B | 174 |
| 1587966 | 17218 | 17237 | 2172 | 2191 | CTGGTGTGACCCCCCCACCC | 58 | B | 175 |
| 1587321 | 17988 | 18007 | 2942 | 2961 | GTTACACGCTTTTACGGAAA | 55 | C | 21 |
| 1587500 | 17998 | 18017 | 2952 | 2971 | TTACACCCTTGTTACACGCT | 95 | C | 176 |
| 1587503 | 16872 | 16891 | 1826 | 1845 | AAGTGGCTTTTCTCTGGAAT | 66 | C | 177 |
| 1587507 | 6296 | 6315 | N/A | N/A | ATTCATTTTTCTCTTGCCCC | 113 | C | 178 |
| 1587519 | 10037 | 10056 | 223 | 242 | TCTTGCTGTCATTTCCGTTT | 103 | C | 179 |
| 1587520 | 5809 | 5828 | N/A | N/A | GCTCTTTTCCTATCAAAACC | 110 | C | 180 |
| 1587527 | 10679 | 10698 | 689 | 708 | TGCAGCACATCCAGGGTCAC | 122 | C | 181 |
| 1587529 | 10159 | 10178 | 345 | 364 | GGTGACCTTCCCAAAGGGCA | 95 | C | 182 |
| 1587547 | 16308 | 16327 | N/A | N/A | CGCTCCTCCACTTTTTTTTT | 89 | C | 183 |
| 1587551 | 5833 | 5852 | N/A | N/A | CCAGGTCCCCTCCACAGCCA | 59 | C | 184 |
| 1587553 | 6304 | 6323 | N/A | N/A | CGTGGCACATTCATTTTTCT | 74 | C | 185 |
| 1587554 | 6713 | 6732 | N/A | N/A | GATGAAAGTACCTCTCCCTC | 74 | C | 186 |
| 1587561 | 5870 | 5889 | N/A | N/A | ATGCCCATCCTAGCCACTCA | 128 | C | 187 |
| 1587563 | 6406 | 6425 | N/A | N/A | TCCCCAGGACCTTCTTCTTC | 99 | C | 188 |
| 1587566 | 16602 | 16621 | 1629 | 1648 | CTGGAAGAACTTGAATCCTT | 95 | C | 189 |
| 1587574 | 14236 | 14255 | N/A | N/A | CCGGGTTTGCTTTATCGGCC | 72 | C | 190 |
| 1587586 | 14417 | 14436 | 1317 | 1336 | GCCGTCCGCCATCTGCACTA | 91 | C | 191 |
| 1587587 | 10128 | 10147 | 314 | 333 | ATGACTTCCCCCTCCGTGAC | 79 | C | 192 |
| 1587589 | 9339 | 9358 | N/A | N/A | ACACTGCTCCTTTTTCCAAA | 97 | C | 193 |
| 1587600 | 15921 | 15940 | N/A | N/A | TGTACCTGCATTTCCAAATA | 111 | C | 194 |
| 1587610 | 17463 | 17482 | 2417 | 2436 | ACTGGAAATATTGCTAGGCA | 37 | C | 195 |
| 1587618 | 17594 | 17613 | 2548 | 2567 | GTAACAGCATACAAGGTAAT | 62 | C | 196 |
| 1587619 | 15912 | 15931 | N/A | N/A | ATTTCCAAATACTTGCATTA | 70 | C | 197 |
| 1587624 | 8846 | 8865 | N/A | N/A | AACAATCTATTCACTGGTTA | 138 | C | 198 |
| 1587631 | 17097 | 17116 | 2051 | 2070 | CCCCACGCCCGACACAGGCC | 93 | C | 199 |
| 1587634 | 8870 | 8889 | N/A | N/A | TGACCTGTCCATTTTCTCTC | 105 | C | 200 |
| 1587644 | 14986 | 15005 | N/A | N/A | GTGTGCATTATACATTCTCA | 44 | C | 201 |
| 1587663 | 16967 | 16986 | 1921 | 1940 | GTGAACTAGATTTAATTTTT | 134 | C | 202 |
| 1587664 | 17734 | 17753 | 2688 | 2707 | CGACACGCCCGCCCCTTCCC | 69 | C | 203 |
| 1587666 | 18009 | 18028 | 2963 | 2982 | ATTATAAATATTTACACCCT | 65 | C | 204 |
| 1587667 | 17217 | 17236 | 2171 | 2190 | TGGTGTGACCCCCCCACCCC | 73 | C | 205 |
| 1587669 | 13464 | 13483 | N/A | N/A | AGTCACACTCCACACACATT | 65† | C | 206 |
| 1587682 | 17560 | 17579 | 2514 | 2533 | TAGATTACACTCATACCATT | 84 | C | 207 |
| 1587687 | 16315 | 16334 | N/A | N/A | AGTACCACGCTCCTCCACTT | 94 | C | 208 |
| 1587688 | 4381 | 4400 | N/A | N/A | ACATCTCCAGCCTCTCCCGC | 61 | C | 209 |
| 1587695 | 5661 | 5680 | N/A | N/A | GAGCAGCTCAGATTCCAGAC | 90 | C | 210 |
| 1587699 | 13406 | 13425 | N/A | N/A | AAGCACCTTCACTCACATGC | 136† | C | 211 |
| 1587703 | 8550 | 8569 | N/A | N/A | GAGCCTCCATCTCCCGTATC | 87 | C | 212 |
| 1587705 | 6587 | 6606 | N/A | N/A | GCTGACTGACCAGAAACCAA | 73 | C | 213 |
| 1587711 | 17241 | 17260 | 2195 | 2214 | CCGGCCAGGCCCCCTGCCTC | 104 | C | 214 |
| 1587716 | 9255 | 9274 | N/A | N/A | CGTACACTATCTCCACGGCC | 120 | C | 215 |
| 1587734 | 13858 | 13877 | N/A | N/A | TGTGACTCTCTTTAGAGACA | 88† | C | 216 |
| 1587736 | 4815 | 4834 | N/A | N/A | CGACCCGACTCTCCCTCCTC | 90 | C | 217 |
| 1587748 | 6256 | 6275 | N/A | N/A | TGCTGCTGTTCTAACCAGAT | 97 | C | 218 |
| 1587750 | 9573 | 9592 | 158 | 177 | ACACAAGTAGAGAAAAGCTC | 73 | C | 219 |
| 1587769 | 8459 | 8478 | N/A | N/A | ATATCACACCCTGTCCCCCC | 78 | C | 220 |
| 1587771 | 16550 | 16569 | 1577 | 1596 | TTGAGATCCTCCTCGGAGAC | 82 | C | 221 |
| 1587772 | 18063 | 18082 | 3017 | 3036 | GTCACCATAAAAAACCGCGC | 61 | C | 222 |
| 1587773 | 12518 | 12537 | 1187 | 1206 | ACCAGCAATACAGAATTTCC | 57† | C | 223 |
| 1587774 | 8792 | 8811 | N/A | N/A | ACTAGACTGATCCTAGGCTC | 68 | C | 224 |
| 1587777 | 15675 | 15694 | N/A | N/A | CTGGTCTTAACTCTTGGGTT | 86 | C | 225 |
| 1587780 | 18086 | 18105 | 3040 | 3059 | CTGTTAGCAAAATATACATT | 74 | C | 226 |
| 1587788 | 16937 | 16956 | 1891 | 1910 | GTAAACTGATTTCTCTTTAA | 76 | C | 227 |
| 1587808 | 18195 | 18214 | 3149 | 3168 | TCAAACGGTTTTAATCGGTT | 53 | C | 228 |
| 1587810 | 12814 | 12833 | N/A | N/A | GCTCCAAGTAATTTTTAGAT | 77† | C | 229 |
| 1587812 | 6380 | 6399 | N/A | N/A | TTCTGACCCCCTTTCCTCAC | 63 | C | 230 |
| 1587815 | 7661 | 7680 | N/A | N/A | CCTGTCTGCTCTCCCCTCCA | 57 | C | 231 |
| 1587825 | 6398 | 6417 | N/A | N/A | ACCTTCTTCTTCTCCAGGTT | 71 | C | 232 |
| 1587829 | 17537 | 17556 | 2491 | 2510 | TCATCTTGCTATAAAAAGTT | 110 | C | 233 |
| 1587833 | 17520 | 17539 | 2474 | 2493 | GTTACTTAAAACTATTTCTT | 124 | C | 234 |
| 1587835 | 17513 | 17532 | 2467 | 2486 | AAAACTATTTCTTTTGCATA | 74 | C | 235 |
| 1587840 | 8884 | 8903 | N/A | N/A | GGCTCCATCACTTCTGACCT | 85 | C | 236 |
| 1587844 | 7245 | 7264 | N/A | N/A | GCGCCTCCACTGCCTTCACA | 75 | C | 237 |
| 1587855 | 9026 | 9045 | N/A | N/A | CCCTCCTCTCCTGTTTTCCA | 60 | C | 238 |
| 1587860 | 17552 | 17571 | 2506 | 2525 | ACTCATACCATTGTATCATC | 47 | C | 239 |
| 1587870 | 8864 | 8883 | N/A | N/A | GTCCATTTTCTCTCCAGAAA | 109 | C | 240 |
| 1587892 | 8103 | 8122 | N/A | N/A | CACCAATCCCACAAGCACTC | 70 | C | 241 |
| 1587894 | 18261 | 18280 | 3215 | 3234 | ACAGACTGGCCAACCTGCCT | 78 | C | 242 |
| 1587895 | 9424 | 9443 | N/A | N/A | CCACACCCACACTTCCACGG | 132 | C | 243 |
| 1587899 | 8431 | 8450 | N/A | N/A | CCCCCAGCTCCACACACCCC | 60 | C | 244 |
| 1587900 | 15737 | 15756 | N/A | N/A | ACTCCTAGCTAATTTTAACC | 67 | C | 245 |
| 1587905 | 17655 | 17674 | 2609 | 2628 | CCCGCCTGCTCTCTGGAAAC | 125 | C | 246 |
| 1587916 | 16000 | 16019 | N/A | N/A | TTGATTTTCTGATAACATTT | 103 | C | 247 |
| 1587920 | 12507 | 12526 | 1176 | 1195 | AGAATTTCCTGCCCCCGCCA | 110† | C | 248 |
| 1587938 | 14834 | 14853 | N/A | N/A | ACGCCAGAAGTCCACACACC | 90 | C | 249 |
| 1587953 | 11866 | 11885 | N/A | N/A | ACTACTAGAGACATCCCTCC | 117 | C | 250 |
| 1587954 | 5417 | 5436 | 119 | 138 | CCAACGGCTATATCTGGGAC | 80 | C | 251 |
| 1587957 | 11445 | 11464 | N/A | N/A | GCACCCACAACCTCCACCGC | 82 | C | 252 |
| 1587321 | 17988 | 18007 | 2942 | 2961 | GTTACACGCTTTTACGGAAA | 68 | D | 21 |
| 1587329 | 17997 | 18016 | 2951 | 2970 | TACACCCTTGTTACACGCTT | 52 | D | 253 |
| 1587528 | 16965 | 16984 | 1919 | 1938 | GAACTAGATTTAATTTTTTT | 98 | D | 254 |
| 1587533 | 10551 | 10570 | 561 | 580 | CTGGACCGAGTTCACCGCCT | 90 | D | 255 |
| 1587536 | 14985 | 15004 | N/A | N/A | TGTGCATTATACATTCTCAC | 86 | D | 256 |
| 1587537 | 8791 | 8810 | N/A | N/A | CTAGACTGATCCTAGGCTCA | 108 | D | 257 |
| 1587541 | 17628 | 17647 | 2582 | 2601 | TGCCTGTGACTTAATTACAA | 81 | D | 258 |
| 1587545 | 6712 | 6731 | N/A | N/A | ATGAAAGTACCTCTCCCTCC | 101 | D | 259 |
| 1587550 | 12517 | 12536 | 1186 | 1205 | CCAGCAATACAGAATTTCCT | 64† | D | 260 |
| 1587559 | 8836 | 8855 | N/A | N/A | TCACTGGTTATTTCTATGTA | 102 | D | 261 |
| 1587565 | 17569 | 17588 | 2523 | 2542 | AAGGAAGTTTAGATTACACT | 80 | D | 262 |
| 1587569 | 16461 | 16480 | N/A | N/A | GCGAGGCTTTCCCCAGACTA | 110 | D | 263 |
| 1587573 | 17216 | 17235 | 2170 | 2189 | GGTGTGACCCCCCCACCCCT | 90 | D | 264 |
| 1587582 | 10136 | 10155 | 322 | 341 | CCAGGGAGATGACTTCCCCC | 87 | D | 265 |
| 1587588 | 17462 | 17481 | 2416 | 2435 | CTGGAAATATTGCTAGGCAC | 79 | D | 266 |
| 1587593 | 11863 | 11882 | N/A | N/A | ACTAGAGACATCCCTCCAAC | 101 | D | 267 |
| 1587596 | 18062 | 18081 | 3016 | 3035 | TCACCATAAAAAACCGCGCC | 115 | D | 268 |
| 1587606 | 18008 | 18027 | 2962 | 2981 | TTATAAATATTTACACCCTT | 106 | D | 269 |
| 1587611 | 14413 | 14432 | 1313 | 1332 | TCCGCCATCTGCACTAGGGC | 134 | D | 270 |
| 1587633 | 16314 | 16333 | N/A | N/A | GTACCACGCTCCTCCACTTT | 38 | D | 271 |
| 1587635 | 8458 | 8477 | N/A | N/A | TATCACACCCTGTCCCCCCT | 93 | D | 272 |
| 1587640 | 9522 | 9541 | N/A | N/A | GCGGAGCTGCACTTCCCACC | 126 | D | 273 |
| 1587643 | 12479 | 12498 | 1148 | 1167 | ATGGCGATCCGACCTGCCGC | 87 | D | 274 |
| 1587653 | 5416 | 5435 | 118 | 137 | CAACGGCTATATCTGGGACA | 90 | D | 275 |
| 1587656 | 6303 | 6322 | N/A | N/A | GTGGCACATTCATTTTTCTC | 93 | D | 276 |
| 1587668 | 6294 | 6313 | N/A | N/A | TCATTTTTCTCTTGCCCCAA | 88 | D | 277 |
| 1587670 | 9961 | 9980 | N/A | N/A | AGGTCCCTGCTATCCCCCTA | 99 | D | 278 |
| 1587686 | 9254 | 9273 | N/A | N/A | GTACACTATCTCCACGGCCA | 91 | D | 279 |
| 1587689 | 16305 | 16324 | N/A | N/A | TCCTCCACTTTTTTTTTTCC | 97 | D | 280 |
| 1587690 | 13405 | 13424 | N/A | N/A | AGCACCTTCACTCACATGCC | 35† | D | 281 |
| 1587693 | 13472 | 13491 | N/A | N/A | GTTCTATCAGTCACACTCCA | 88† | D | 282 |
| 1587696 | 4379 | 4398 | N/A | N/A | ATCTCCAGCCTCTCCCGCTT | 104 | D | 283 |
| 1587704 | 17240 | 17259 | 2194 | 2213 | CGGCCAGGCCCCCTGCCTCT | 104 | D | 284 |
| 1587707 | 10058 | 10077 | 244 | 263 | TTCGGCTGTCACCTTTGAAC | 100 | D | 285 |
| 1587722 | 6254 | 6273 | N/A | N/A | CTGCTGTTCTAACCAGATGC | 77 | D | 286 |
| 1587738 | 8869 | 8888 | N/A | N/A | GACCTGTCCATTTTCTCTCC | 103 | D | 287 |
| 1587746 | 16773 | 16792 | 1727 | 1746 | TGGTGGTTCTCCCCGAGGTC | 87 | D | 288 |
| 1587751 | 6379 | 6398 | N/A | N/A | TCTGACCCCCTTTCCTCACA | 103 | D | 289 |
| 1587760 | 6397 | 6416 | N/A | N/A | CCTTCTTCTTCTCCAGGTTC | 67 | D | 290 |
| 1587763 | 14832 | 14851 | N/A | N/A | GCCAGAAGTCCACACACCGA | 49 | D | 291 |
| 1587768 | 6470 | 6489 | N/A | N/A | ACCAGCAGAATCATTAACCA | 87 | D | 292 |
| 1587778 | 8863 | 8882 | N/A | N/A | TCCATTTTCTCTCCAGAAAC | 103 | D | 293 |
| 1587783 | 5808 | 5827 | N/A | N/A | CTCTTTTCCTATCAAAACCA | 112 | D | 294 |
| 1587791 | 14109 | 14128 | N/A | N/A | GCATATGCCACCGACAGGTC | 105 | D | 295 |
| 1587794 | 15736 | 15755 | N/A | N/A | CTCCTAGCTAATTTTAACCA | 118 | D | 296 |
| 1587802 | 18194 | 18213 | 3148 | 3167 | CAAACGGTTTTAATCGGTTC | 67 | D | 297 |
| 1587803 | 8339 | 8358 | N/A | N/A | GCTTGGTCTTCTCTTAAGGC | 95 | D | 298 |
| 1587813 | 5814 | 5833 | N/A | N/A | AGGTGGCTCTTTTCCTATCA | 96 | D | 299 |
| 1587824 | 7111 | 7130 | N/A | N/A | CCTAGGTGTCTTTAACCCCA | 74 | D | 300 |
| 1587827 | 7658 | 7677 | N/A | N/A | GTCTGCTCTCCCCTCCAGCT | 118 | D | 301 |
| 1587830 | 15908 | 15927 | N/A | N/A | CCAAATACTTGCATTATGTA | 76 | D | 302 |
| 1587832 | 10850 | 10869 | 734 | 753 | AAGGTGATGATCTTCAACAC | 44 | D | 303 |
| 1587838 | 6405 | 6424 | N/A | N/A | CCCCAGGACCTTCTTCTTCT | 112 | D | 304 |
| 1587845 | 7991 | 8010 | N/A | N/A | CTGTTCAGTCCCCAAGACTT | 63 | D | 305 |
| 1587847 | 8881 | 8900 | N/A | N/A | TCCATCACTTCTGACCTGTC | 91 | D | 306 |
| 1587853 | 17504 | 17523 | 2458 | 2477 | TCTTTTGCATATAAATGAAA | 113 | D | 307 |
| 1587863 | 15450 | 15469 | N/A | N/A | GGGCACCTATAGTCCCTCCC | 87 | D | 308 |
| 1587865 | 4486 | 4505 | N/A | N/A | GCTGCGCCTCTTCTGACACC | 92 | D | 309 |
| 1587868 | 18085 | 18104 | 3039 | 3058 | TGTTAGCAAAATATACATTT | 54 | D | 310 |
| 1587869 | 9022 | 9041 | N/A | N/A | CCTCTCCTGTTTTCCAGCCC | 93 | D | 311 |
| 1587873 | 15996 | 16015 | N/A | N/A | TTTTCTGATAACATTTCACA | 90 | D | 312 |
| 1587875 | 8545 | 8564 | N/A | N/A | TCCATCTCCCGTATCAGGAT | 109 | D | 313 |
| 1587882 | 9338 | 9357 | N/A | N/A | CACTGCTCCTTTTTCCAAAT | 102 | D | 314 |
| 1587884 | 17028 | 17047 | 1982 | 2001 | AACTCCCGCTGCCACAGTCA | 100 | D | 315 |
| 1587887 | 12813 | 12832 | N/A | N/A | CTCCAAGTAATTTTTAGATT | 78† | D | 316 |
| 1587891 | 18260 | 18279 | 3214 | 3233 | CAGACTGGCCAACCTGCCTC | 93 | D | 317 |
| 1587897 | 17551 | 17570 | 2505 | 2524 | CTCATACCATTGTATCATCT | 42 | D | 318 |
| 1587906 | 16596 | 16615 | 1623 | 1642 | GAACTTGAATCCTTTGACGA | 106 | D | 319 |
| 1587909 | 17536 | 17555 | 2490 | 2509 | CATCTTGCTATAAAAAGTTA | 88 | D | 320 |
| 1587919 | 17733 | 17752 | 2687 | 2706 | GACACGCCCGCCCCTTCCCT | 97 | D | 321 |
| 1587925 | 17519 | 17538 | 2473 | 2492 | TTACTTAAAACTATTTCTTT | 83 | D | 322 |
| 1587928 | 13463 | 13482 | N/A | N/A | GTCACACTCCACACACATTC | 83† | D | 323 |
| 1587935 | 17558 | 17577 | 2512 | 2531 | GATTACACTCATACCATTGT | 80 | D | 324 |
| 1587937 | 15920 | 15939 | N/A | N/A | GTACCTGCATTTCCAAATAC | 115 | D | 325 |
| 1587945 | 9423 | 9442 | N/A | N/A | CACACCCACACTTCCACGGC | 121 | D | 326 |
| 1587961 | 5846 | 5865 | N/A | N/A | GGCTGGATTCTATCCAGGTC | 113 | D | 327 |
| 1587962 | 5660 | 5679 | N/A | N/A | AGCAGCTCAGATTCCAGACC | 107 | D | 328 |
| 1587963 | 16936 | 16955 | 1890 | 1909 | TAAACTGATTTCTCTTTAAA | 94 | D | 329 |
| 1587321 | 17988 | 18007 | 2942 | 2961 | GTTACACGCTTTTACGGAAA | 48 | E | 21 |
| 1587502 | 17996 | 18015 | 2950 | 2969 | ACACCCTTGTTACACGCTTT | 49 | E | 330 |
| 1587512 | 6301 | 6320 | N/A | N/A | GGCACATTCATTTTTCTCTT | 62 | E | 331 |
| 1587513 | 16304 | 16323 | N/A | N/A | CCTCCACTTTTTTTTTTCCA | 86 | E | 332 |
| 1587517 | 5571 | 5590 | N/A | N/A | GCCCAACTCCAGATTCCAGA | 116 | E | 333 |
| 1587531 | 10057 | 10076 | 243 | 262 | TCGGCTGTCACCTTTGAACT | 39 | E | 334 |
| 1587532 | 17434 | 17453 | 2388 | 2407 | GCAACTGCAAAGTATATAAT | 63 | E | 335 |
| 1587544 | 13351 | 13370 | N/A | N/A | GCCGATCCCAACCACGCCCC | 33† | E | 336 |
| 1587548 | 12513 | 12532 | 1182 | 1201 | CAATACAGAATTTCCTGCCC | 61† | E | 337 |
| 1587549 | 9422 | 9441 | N/A | N/A | ACACCCACACTTCCACGGCA | 103 | E | 338 |
| 1587567 | 17547 | 17566 | 2501 | 2520 | TACCATTGTATCATCTTGCT | 24 | E | 339 |
| 1587571 | 16456 | 16475 | N/A | N/A | GCTTTCCCCAGACTAGTTTT | 65 | E | 340 |
| 1587572 | 17215 | 17234 | 2169 | 2188 | GTGTGACCCCCCCACCCCTT | 63 | E | 341 |
| 1587579 | 7109 | 7128 | N/A | N/A | TAGGTGTCTTTAACCCCACA | 45 | E | 342 |
| 1587592 | 10134 | 10153 | 320 | 339 | AGGGAGATGACTTCCCCCTC | 94 | E | 343 |
| 1587594 | 8467 | 8486 | N/A | N/A | ACCAGTGGATATCACACCCT | 61 | E | 344 |
| 1587598 | 10308 | 10327 | 411 | 430 | GTTGGCAGCCTCCTCCGTGT | 73 | E | 345 |
| 1587603 | 6466 | 6485 | N/A | N/A | GCAGAATCATTAACCAACTA | 30 | E | 346 |
| 1587607 | 17730 | 17749 | 2684 | 2703 | ACGCCCGCCCCTTCCCTGAA | 80 | E | 347 |
| 1587628 | 6252 | 6271 | N/A | N/A | GCTGTTCTAACCAGATGCCA | 82 | E | 348 |
| 1587638 | 8862 | 8881 | N/A | N/A | CCATTTTCTCTCCAGAAACA | 94 | E | 349 |
| 1587648 | 18081 | 18100 | 3035 | 3054 | AGCAAAATATACATTTGTGT | 82 | E | 350 |
| 1587650 | 9253 | 9272 | N/A | N/A | TACACTATCTCCACGGCCAA | 90 | E | 351 |
| 1587665 | 14039 | 14058 | N/A | N/A | ACCAAATAAAATTCGCAAAC | 50 | E | 352 |
| 1587672 | 13462 | 13481 | N/A | N/A | TCACACTCCACACACATTCC | 37† | E | 353 |
| 1587677 | 8457 | 8476 | N/A | N/A | ATCACACCCTGTCCCCCCTC | 98 | E | 354 |
| 1587680 | 16593 | 16612 | 1620 | 1639 | CTTGAATCCTTTGACGACGC | 91 | E | 355 |
| 1587685 | 18257 | 18276 | 3211 | 3230 | ACTGGCCAACCTGCCTCTAC | 98 | E | 356 |
| 1587692 | 17025 | 17044 | 1979 | 1998 | TCCCGCTGCCACAGTCACCA | 82 | E | 357 |
| 1587697 | 17564 | 17583 | 2518 | 2537 | AGTTTAGATTACACTCATAC | 69 | E | 358 |
| 1587700 | 10847 | 10866 | 731 | 750 | GTGATGATCTTCAACACTGT | 62 | E | 359 |
| 1587702 | 5813 | 5832 | N/A | N/A | GGTGGCTCTTTTCCTATCAA | 63 | E | 360 |
| 1587706 | 8835 | 8854 | N/A | N/A | CACTGGTTATTTCTATGTAA | 57 | E | 361 |
| 1587712 | 15896 | 15915 | N/A | N/A | ATTATGTAATTTTTACAGAC | 117 | E | 362 |
| 1587731 | 4034 | 4053 | N/A | N/A | GCGCCAGCTTTCCCTCCCGG | 66 | E | 363 |
| 1587747 | 16313 | 16332 | N/A | N/A | TACCACGCTCCTCCACTTTT | 91 | E | 364 |
| 1587752 | 8992 | 9011 | N/A | N/A | CCCGACCTCCGTCTCTGCAC | 68 | E | 365 |
| 1587761 | 12782 | 12801 | N/A | N/A | ACCAAAAAATAATTCATTCT | 138† | E | 366 |
| 1587767 | 8868 | 8887 | N/A | N/A | ACCTGTCCATTTTCTCTCCA | 69 | E | 367 |
| 1587775 | 9337 | 9356 | N/A | N/A | ACTGCTCCTTTTTCCAAATT | 122 | E | 368 |
| 1587776 | 5845 | 5864 | N/A | N/A | GCTGGATTCTATCCAGGTCC | 105 | E | 369 |
| 1587781 | 18006 | 18025 | 2960 | 2979 | ATAAATATTTACACCCTTGT | 89 | E | 370 |
| 1587792 | 18047 | 18066 | 3001 | 3020 | GCGCCGCCGCCCCTCGGGTC | 82 | E | 371 |
| 1587793 | 8880 | 8899 | N/A | N/A | CCATCACTTCTGACCTGTCC | 80 | E | 372 |
| 1587799 | 8338 | 8357 | N/A | N/A | CTTGGTCTTCTCTTAAGGCC | 98 | E | 373 |
| 1587800 | 8787 | 8806 | N/A | N/A | ACTGATCCTAGGCTCAGGAC | 95 | E | 374 |
| 1587804 | 17625 | 17644 | 2579 | 2598 | CTGTGACTTAATTACAAGGA | 61 | E | 375 |
| 1587805 | 17518 | 17537 | 2472 | 2491 | TACTTAAAACTATTTCTTTT | 65 | E | 376 |
| 1587807 | 13470 | 13489 | N/A | N/A | TCTATCAGTCACACTCCACA | 111† | E | 377 |
| 1587811 | 14984 | 15003 | N/A | N/A | GTGCATTATACATTCTCACA | 52 | E | 378 |
| 1587816 | 17529 | 17548 | 2483 | 2502 | CTATAAAAAGTTACTTAAAA | 70 | E | 379 |
| 1587817 | 9843 | 9862 | N/A | N/A | CCCGACAGCTCCTGACAACC | 116 | E | 380 |
| 1587820 | 7657 | 7676 | N/A | N/A | TCTGCTCTCCCCTCCAGCTC | 89 | E | 381 |
| 1587826 | 9521 | 9540 | N/A | N/A | CGGAGCTGCACTTCCCACCA | 134 | E | 382 |
| 1587831 | 4483 | 4502 | N/A | N/A | GCGCCTCTTCTGACACCACA | 47 | E | 383 |
| 1587841 | 6291 | 6310 | N/A | N/A | TTTTTCTCTTGCCCCAAACA | 76 | E | 384 |
| 1587850 | 6404 | 6423 | N/A | N/A | CCCAGGACCTTCTTCTTCTC | 68 | E | 385 |
| 1587857 | 17236 | 17255 | 2190 | 2209 | CAGGCCCCCTGCCTCTCTCT | 132 | E | 386 |
| 1587871 | 6708 | 6727 | N/A | N/A | AAGTACCTCTCCCTCCTCCC | 86 | E | 387 |
| 1587881 | 16731 | 16750 | 1685 | 1704 | GCCTGGACCGCCTCCTCCAC | 74 | E | 388 |
| 1587890 | 11745 | 11764 | N/A | N/A | GCCTCCACAGCCCTGTCCTA | 63 | E | 389 |
| 1587896 | 6386 | 6405 | N/A | N/A | TCCAGGTTCTGACCCCCTTT | 81 | E | 390 |
| 1587903 | 17557 | 17576 | 2511 | 2530 | ATTACACTCATACCATTGTA | 109 | E | 391 |
| 1587904 | 16935 | 16954 | 1889 | 1908 | AAACTGATTTCTCTTTAAAA | 82 | E | 392 |
| 1587908 | 15918 | 15937 | N/A | N/A | ACCTGCATTTCCAAATACTT | 68 | E | 393 |
| 1587915 | 5807 | 5826 | N/A | N/A | TCTTTTCCTATCAAAACCAA | 92 | E | 394 |
| 1587917 | 15995 | 16014 | N/A | N/A | TTTCTGATAACATTTCACAA | 89 | E | 395 |
| 1587918 | 5117 | 5136 | N/A | N/A | TTCCAAGACAAAGACACCCC | 95 | E | 396 |
| 1587924 | 14796 | 14815 | N/A | N/A | AGCAGAGCCCTTGCCCGCCC | 81 | E | 397 |
| 1587926 | 17503 | 17522 | 2457 | 2476 | CTTTTGCATATAAATGAAAA | 21 | E | 398 |
| 1587931 | 15380 | 15399 | N/A | N/A | ACTTGCAGAGATCCCGCCAC | 59 | E | 399 |
| 1587934 | 16964 | 16983 | 1918 | 1937 | AACTAGATTTAATTTTTTTA | 65 | E | 400 |
| 1587936 | 7890 | 7909 | N/A | N/A | ACAGACCCTCGAGTCCTCTC | 93 | E | 401 |
| 1587939 | 14393 | 14412 | 1293 | 1312 | GTTCTCCTTCTTATTGAACA | 85† | E | 402 |
| 1587942 | 18113 | 18132 | 3067 | 3086 | GCGGTCACAATACTGAGCCT | 99 | E | 403 |
| 1587947 | 6378 | 6397 | N/A | N/A | CTGACCCCCTTTCCTCACAT | 91 | E | 404 |
| 1587948 | 12478 | 12497 | 1147 | 1166 | TGGCGATCCGACCTGCCGCC | 93 | E | 405 |
| 1587959 | 15735 | 15754 | N/A | N/A | TCCTAGCTAATTTTAACCAA | 87 | E | 406 |
| 1587321 | 17988 | 18007 | 2942 | 2961 | GTTACACGCTTTTACGGAAA | 65 | F | 21 |
| 1587506 | 17990 | 18009 | 2944 | 2963 | TTGTTACACGCTTTTACGGA | 55 | F | 407 |
| 1587510 | 17546 | 17565 | 2500 | 2519 | ACCATTGTATCATCTTGCTA | 28 | F | 408 |
| 1587516 | 5812 | 5831 | N/A | N/A | GTGGCTCTTTTCCTATCAAA | 82 | F | 409 |
| 1587518 | 13942 | 13961 | N/A | N/A | GCATCAAAATCATCTCAATT | 74 | F | 410 |
| 1587521 | 12512 | 12531 | 1181 | 1200 | AATACAGAATTTCCTGCCCC | 49† | F | 411 |
| 1587524 | 9345 | 9364 | N/A | N/A | ACCAAAACACTGCTCCTTTT | 71 | F | 412 |
| 1587525 | 14388 | 14407 | 1288 | 1307 | CCTTCTTATTGAACAGGATC | 69† | F | 413 |
| 1587530 | 6299 | 6318 | N/A | N/A | CACATTCATTTTTCTCTTGC | 58 | F | 414 |
| 1587534 | 17563 | 17582 | 2517 | 2536 | GTTTAGATTACACTCATACC | 89 | F | 415 |
| 1587539 | 10048 | 10067 | 234 | 253 | ACCTTTGAACTTCTTGCTGT | 88 | F | 416 |
| 1587542 | 9054 | 9073 | N/A | N/A | GCGCTGCCTCCCTCACTCTC | 64 | F | 417 |
| 1587552 | 5748 | 5767 | N/A | N/A | ACGGCACGATTCCAGCCTCT | 90 | F | 418 |
| 1587564 | 17556 | 17575 | 2510 | 2529 | TTACACTCATACCATTGTAT | 105 | F | 419 |
| 1587583 | 10133 | 10152 | 319 | 338 | GGGAGATGACTTCCCCCTCC | 68 | F | 420 |
| 1587584 | 6462 | 6481 | N/A | N/A | AATCATTAACCAACTACCCC | 105 | F | 421 |
| 1587591 | 4846 | 4865 | N/A | N/A | GCCTCAGTTACTGATCAACC | 61 | F | 422 |
| 1587602 | 11494 | 11513 | 1001 | 1020 | GCTGAGATTATACCAGGTGC | 71 | F | 423 |
| 1587613 | 17233 | 17252 | 2187 | 2206 | GCCCCCTGCCTCTCTCTGGT | 69 | F | 424 |
| 1587615 | 9500 | 9519 | N/A | N/A | GCCAGAGCCTCCCTTCCCCA | 110 | F | 425 |
| 1587617 | 8456 | 8475 | N/A | N/A | TCACACCCTGTCCCCCCTCA | 60 | F | 426 |
| 1587620 | 17024 | 17043 | 1978 | 1997 | CCCGCTGCCACAGTCACCAA | 49 | F | 427 |
| 1587625 | 14940 | 14959 | N/A | N/A | TCCAAAGTGTCCAACGACCC | 68 | F | 428 |
| 1587632 | 16302 | 16321 | N/A | N/A | TCCACTTTTTTTTTTCCAGA | 54 | F | 429 |
| 1587636 | 12476 | 12495 | 1145 | 1164 | GCGATCCGACCTGCCGCCGC | 117 | F | 430 |
| 1587641 | 17726 | 17745 | 2680 | 2699 | CCGCCCCTTCCCTGAAGCTC | 71 | F | 431 |
| 1587647 | 18109 | 18128 | 3063 | 3082 | TCACAATACTGAGCCTGGAA | 90 | F | 432 |
| 1587649 | 14654 | 14673 | 1461 | 1480 | GTTGCCGTAGTCCTTGGTCA | 136 | F | 433 |
| 1587651 | 9336 | 9355 | N/A | N/A | CTGCTCCTTTTTCCAAATTC | 88 | F | 434 |
| 1587655 | 9842 | 9861 | N/A | N/A | CCGACAGCTCCTGACAACCC | 93 | F | 435 |
| 1587659 | 13469 | 13488 | N/A | N/A | CTATCAGTCACACTCCACAC | 85† | F | 436 |
| 1587675 | 17432 | 17451 | 2386 | 2405 | AACTGCAAAGTATATAATAC | 77 | F | 437 |
| 1587676 | 17491 | 17510 | 2445 | 2464 | AATGAAAAAAGATTAGAATA | 97 | F | 438 |
| 1587681 | 5570 | 5589 | N/A | N/A | CCCAACTCCAGATTCCAGAC | 71 | F | 439 |
| 1587694 | 8852 | 8871 | N/A | N/A | TCCAGAAACAATCTATTCAC | 63 | F | 440 |
| 1587698 | 17516 | 17535 | 2470 | 2489 | CTTAAAACTATTTCTTTTGC | 64 | F | 441 |
| 1587701 | 6289 | 6308 | N/A | N/A | TTTCTCTTGCCCCAAACACC | 116 | F | 442 |
| 1587719 | 16455 | 16474 | N/A | N/A | CTTTCCCCAGACTAGTTTTC | 79 | F | 443 |
| 1587728 | 7656 | 7675 | N/A | N/A | CTGCTCTCCCCTCCAGCTCA | 88 | F | 444 |
| 1587735 | 18256 | 18275 | 3210 | 3229 | CTGGCCAACCTGCCTCTACA | 80 | F | 445 |
| 1587739 | 7884 | 7903 | N/A | N/A | CCTCGAGTCCTCTCGCTTTC | 71 | F | 446 |
| 1587740 | 10307 | 10326 | 410 | 429 | TTGGCAGCCTCCTCCGTGTT | 103 | F | 447 |
| 1587749 | 4033 | 4052 | N/A | N/A | CGCCAGCTTTCCCTCCCGGC | 59 | F | 448 |
| 1587753 | 13000 | 13019 | N/A | N/A | TGGGCCTCTTGTTTCACCCA | 130† | F | 449 |
| 1587766 | 18035 | 18054 | 2989 | 3008 | CTCGGGTCTCACAACAGGTA | 59 | F | 450 |
| 1587770 | 8991 | 9010 | N/A | N/A | CCGACCTCCGTCTCTGCACA | 94 | F | 451 |
| 1587784 | 15895 | 15914 | N/A | N/A | TTATGTAATTTTTACAGACA | 68 | F | 452 |
| 1587789 | 8867 | 8886 | N/A | N/A | CCTGTCCATTTTCTCTCCAG | 91 | F | 453 |
| 1587795 | 5844 | 5863 | N/A | N/A | CTGGATTCTATCCAGGTCCC | 88 | F | 454 |
| 1587809 | 8463 | 8482 | N/A | N/A | GTGGATATCACACCCTGTCC | 61 | F | 455 |
| 1587818 | 6402 | 6421 | N/A | N/A | CAGGACCTTCTTCTTCTCCA | 88 | F | 456 |
| 1587819 | 6707 | 6726 | N/A | N/A | AGTACCTCTCCCTCCTCCCA | 104 | F | 457 |
| 1587834 | 8834 | 8853 | N/A | N/A | ACTGGTTATTTCTATGTAAT | 90 | F | 458 |
| 1587836 | 6383 | 6402 | N/A | N/A | AGGTTCTGACCCCCTTTCCT | 96 | F | 459 |
| 1587849 | 16934 | 16953 | 1888 | 1907 | AACTGATTTCTCTTTAAAAA | 63 | F | 460 |
| 1587852 | 6375 | 6394 | N/A | N/A | ACCCCCTTTCCTCACATGCT | 73 | F | 461 |
| 1587856 | 16577 | 16596 | 1604 | 1623 | ACGCCCCCATTGCTGGAAAA | 58 | F | 462 |
| 1587859 | 15992 | 16011 | N/A | N/A | CTGATAACATTTCACAAGTA | 114 | F | 463 |
| 1587861 | 16941 | 16960 | 1895 | 1914 | ACAGGTAAACTGATTTCTCT | 41 | F | 464 |
| 1587866 | 15916 | 15935 | N/A | N/A | CTGCATTTCCAAATACTTGC | 74 | F | 465 |
| 1587867 | 8766 | 8785 | N/A | N/A | GCAGACATCCCCCCCGCTGC | 109 | F | 466 |
| 1587874 | 16715 | 16734 | 1669 | 1688 | CCACGGAGCCCATCTGGATC | 55 | F | 467 |
| 1587876 | 10845 | 10864 | 729 | 748 | GATGATCTTCAACACTGTGC | 113 | F | 468 |
| 1587877 | 15733 | 15752 | N/A | N/A | CTAGCTAATTTTAACCAATT | 75 | F | 469 |
| 1587883 | 12707 | 12726 | N/A | N/A | ACTCAAGCCATCTTGGAAAA | 107† | F | 470 |
| 1587885 | 17624 | 17643 | 2578 | 2597 | TGTGACTTAATTACAAGGAA | 72 | F | 471 |
| 1587889 | 16312 | 16331 | N/A | N/A | ACCACGCTCCTCCACTTTTT | 79 | F | 472 |
| 1587901 | 8879 | 8898 | N/A | N/A | CATCACTTCTGACCTGTCCA | 52 | F | 473 |
| 1587923 | 14989 | 15008 | N/A | N/A | AATGTGTGCATTATACATTC | 82 | F | 474 |
| 1587930 | 17523 | 17542 | 2477 | 2496 | AAAGTTACTTAAAACTATTT | 58 | F | 475 |
| 1587933 | 6243 | 6262 | N/A | N/A | ACCAGATGCCATCAAAGATA | 90 | F | 476 |
| 1587940 | 7108 | 7127 | N/A | N/A | AGGTGTCTTTAACCCCACAC | 85 | F | 477 |
| 1587941 | 8328 | 8347 | N/A | N/A | TCTTAAGGCCGCCTCCCCTC | 61 | F | 478 |
| 1587949 | 18004 | 18023 | 2958 | 2977 | AAATATTTACACCCTTGTTA | 101 | F | 479 |
| 1587951 | 17214 | 17233 | 2168 | 2187 | TGTGACCCCCCCACCCCTTG | 65 | F | 480 |
| 1587952 | 13458 | 13477 | N/A | N/A | ACTCCACACACATTCCAGTT | 107† | F | 481 |
| 1587958 | 4469 | 4488 | N/A | N/A | ACCACAGACTGCACGGACCA | 74 | F | 482 |
| 1587960 | 18080 | 18099 | 3034 | 3053 | GCAAAATATACATTTGTGTC | 57 | F | 483 |

1. An oligomeric compound comprising a modified oligonucleotide consisting of 8 to 80 linked nucleosides, wherein the nucleobase sequence of the modified oligonucleotide is at least 80% complementary to the nucleobase sequence of an equal length portion of a PTBP1 nucleic acid, and wherein the modified oligonucleotide has at least one modification selected from a modified sugar moiety and a modified internucleoside linkage.

2. The oligomeric compound of claim 1, wherein the PTBP1 nucleic acid has the nucleobase sequence of SEQ ID NO: 1 or SEQ ID NO: 2.

3. The oligomeric compound of claim 1 or claim 2, wherein the nucleobase sequence of the modified oligonucleotide is at least 80% complementary to the nucleobase sequence of an equal length portion within nucleobases 16,938-16,960; nucleobases 17,538-17,573; or nucleobases 17,988-18,016 of SEQ ID NO: 1.

4. The oligomeric compound of any of claims 1-3, wherein the nucleobase sequence of the modified oligonucleotide is at least 85%, at least 90%, at least 95%, or 100% complementary to the nucleobase sequence of an equal length portion of the PTBP1 nucleic acid.

5. An oligomeric compound, wherein the oligomeric compound comprises a modified oligonucleotide consisting of 8 to 80 linked nucleosides, wherein the nucleobase sequence of the modified oligonucleotide comprises at least 8, at least 9, at least 10, at least 11, at least 12, at least 13, at least 14, at least 15, at least 16, at least 17, at least 18, at least 19, or 20 contiguous nucleobases of any of the nucleobase sequences of SEQ ID NOs: 21-483, and wherein the modified oligonucleotide comprises at least one modification selected from a modified sugar moiety and a modified internucleoside linkage.

6. The oligomeric compound of claim 5, wherein the nucleobase sequence of the modified oligonucleotide comprises the nucleobase sequence of any of SEQ ID NOs: 21-483.

7. The oligomeric compound of claim 6, wherein the modified oligonucleotide has a nucleobase sequence consisting of the nucleobase sequence of any of SEQ ID NOs: 21-483.

8. The oligomeric compound of any of claims 5-7, wherein the modified oligonucleotide has a nucleobase sequence comprising at least 8, at least 9, at least 10, at least 11, at least 12, at least 13, at least 14, at least 15, at least 16, at least 17, at least 18, at least 19, or 20 contiguous nucleobases of the nucleobase sequence of
SEQ ID NO: 21, 253, 330, or 407;
SEQ ID NO: 84, 145, 170, 239, 318, 339, or 408; or
SEQ ID NO: 21, 253, 330, or 407.

9. The oligomeric compound of claim 8, wherein the modified oligonucleotide consists of 10-80, 20 to 80, 10-50, 20-50, 10-30, or 20-30 linked nucleosides, wherein the nucleobase sequence of the modified oligonucleotide comprises the nucleobase sequence of
SEQ ID NO: 21, 253, 330, or 407;
SEQ ID NO: 84, 145, 170, 239, 318, 339, or 408; or
SEQ ID NO: 21, 253, 330, or 407.

10. The oligomeric compound of claim 9, wherein the modified oligonucleotide has a nucleobase sequence consisting of the nucleobase sequence of
SEQ ID NO: 21, 253, 330, or 407;
SEQ ID NO: 84, 145, 170, 239, 318, 339, or 408; or
SEQ ID NO: 21, 253, 330, or 407.

11. The oligomeric compound of any of claims 5-10, wherein the nucleobase sequence of the modified oligonucleotide is at least 85%, at least 90%, at least 95%, or 100% complementary to the nucleobase sequence of an equal length portion of a PTBP1 nucleic acid, wherein the PTBP1 nucleic acid has the nucleobase sequence of SEQ ID NO: 1 or SEQ ID NO: 2.

12. The oligomeric compound of any of claims 1-11, wherein the modified oligonucleotide consists of 10 to 25, 10 to 30, 10 to 50, 12 to 20, 12 to 25, 12 to 30, 12 to 50, 13 to 20, 13 to 25, 13 to 30, 13 to 50, 14 to 20, 14 to 25, 14 to 30, 14 to 50, 15 to 20, 15 to 25, 15 to 30, 15 to 50, 16 to 18, 16 to 20, 16 to 25, 16 to 30, 16 to 50, 17 to 20, 17 to 25, 17 to 30, 17 to 50, 18 to 20, 18 to 22, 18 to 25, 18 to 30, 18 to 50, 19 to 20, 19 to 25, 19 to 30, 19 to 50, 20 to 25, 20 to 30, 20 to 50, 21 to 25, 21 to 30, 21 to 50, 22 to 25, 22 to 30, 22 to 50, 23 to 25, 23 to 30, or 23 to 50 linked nucleosides.

13. The oligomeric compound of any of claims 1-12, wherein the modified oligonucleotide consists of 20 linked nucleosides.

14. The oligomeric compound of any of claims 1-13, wherein at least one nucleoside of the modified oligonucleotide comprises a modified sugar moiety.

15. The oligomeric compound of claim 14, wherein the modified sugar moiety comprises a bicyclic sugar moiety.

16. The oligomeric compound of claim 15, wherein the bicyclic sugar moiety comprises a 2′-4′ bridge selected from —O—CH₂—; and —O—CH(CH₃)—.

17. The oligomeric compound of claim 14, wherein the modified sugar moiety comprises a non-bicyclic modified sugar moiety.

18. The oligomeric compound of claim 17, wherein the non-bicyclic modified sugar moiety is a 2′-MOE sugar moiety or 2′-OMe sugar moiety.

19. The oligomeric compound of any of claims 1-18, wherein at least one nucleoside of the modified oligonucleotide compound comprises a sugar surrogate.

20. The oligomeric compound of any of claims 1-19, wherein the modified oligonucleotide comprises at least one modified internucleoside linkage.

21. The oligomeric compound of claim 20, wherein at least one modified internucleoside linkage is a phosphorothioate internucleoside linkage.

22. The oligomeric compound of claim 20 or claim 21, wherein each internucleoside linkage is a modified internucleoside linkage.

23. The oligomeric compound of claim 22, wherein each internucleoside linkage is a phosphorothioate internucleoside linkage.

24. The oligomeric compound of claim 20 or claim 21, wherein at least one internucleoside linkage of the modified oligonucleotide is a phosphodiester internucleoside linkage.

25. The oligomeric compound of any of claims 1-21 or 24, wherein each internucleoside linkage of the modified oligonucleotide is independently selected from a phosphodiester internucleoside linkage or a phosphorothioate internucleoside linkage.

26. The oligomeric compound of any of claims 1-21 or 24-25, wherein at least 10, at least 11, at least 12, at least 13, at least 14, at least 15, at least 16, at least 17, or at least 18 internucleoside linkages of the modified oligonucleotide are phosphorothioate internucleoside linkages.

27. The oligomeric compound of any of claims 1-21 or 24-26, wherein the internucleoside linkage motif of the modified oligonucleotide is 5′-sooosssssssssssooss-3′, wherein each “s” represents a phosphorothioate internucleoside linkage and each “o” represents a phosphodiester internucleoside linkage.

28. The oligomeric compound of any of claims 1-27, wherein the modified oligonucleotide comprises at least one modified nucleobase.

29. The oligomeric compound of claim 28, wherein the modified nucleobase is 5-methylcytosine.

30. The oligomeric compound of claim 29, wherein each cytosine is a 5-methylcytosine.

31. The oligomeric compound of any of claims 1-30, wherein the modified oligonucleotide comprises a deoxy region.

32. The oligomeric compound of claim 31, wherein each nucleoside of the deoxy region is a 2′-β-D-deoxynucleoside.

33. The oligomeric compound of claim 31 or claim 32, wherein the deoxy region consists of 6, 7, 8, 9, 10, or 6-10 linked nucleosides.

34. The oligomeric compound of any of claims 31-33, wherein each nucleoside immediately adjacent to the deoxy region comprises a modified sugar moiety.

35. The oligomeric compound of any of claims 31-34, wherein the deoxy region is flanked on the 5′-side by a 5′ external region consisting of 1-6 linked 5′ external region nucleosides and on the 3′-side by a 3′ external region consisting of 1-6 linked 3′ external region nucleosides; wherein
the 3′-most nucleoside of the 5′ external region comprises a modified sugar moiety; and
the 5′-most nucleoside of the 3′ external region comprises a modified sugar moiety.

36. The oligomeric compound of claim 35, wherein each nucleoside of the 3′ external region comprises a modified sugar moiety.

37. The oligomeric compound of claim 35 or claim 36, wherein each nucleoside of the 5′ external region comprises a modified sugar moiety.

38. The oligomeric compound of claim 37, wherein the modified oligonucleotide has:
a 5′ external region consisting of 5 linked nucleosides;
a deoxy region consisting of 10 linked nucleosides; and
a 3′ external region consisting of 5 linked nucleosides;
wherein each of the 5′ external region nucleosides and each of the 3′ external region nucleosides is a 2′-MOE nucleoside.

39. The oligomeric compound of claim 37, wherein the modified oligonucleotide has:
a 5′ external region consisting of 1-6 linked nucleosides;
a deoxy region consisting of 6-10 linked nucleosides; and
a 3′ external region consisting of 1-6 linked nucleosides;
wherein each of the 5′ external region nucleosides and each of the 3′ external region nucleosides is a cEt nucleoside or a 2′-MOE nucleoside; and each of the deoxy region nucleosides is a 2′-β-D-deoxynucleoside.

40. The oligomeric compound of any of claims 1-39, wherein the modified oligonucleotide has a sugar motif of 5′-eeeeeddddddddddeeeee-3′, wherein each “d” represents a 2′-β-D-deoxyribosyl sugar moiety, and each “e” represents a 2′-MOE sugar moiety.

41. The oligomeric compound of any of claims 1-40, consisting of the modified oligonucleotide.

42. The oligomeric compound of any of claims 1-40, wherein the oligomeric compound comprises a conjugate group.

43. The oligomeric compound of claim 42, wherein the conjugate group comprises a conjugate linker and a conjugate moiety.

44. The oligomeric compound of claim 43, wherein the conjugate linker consists of a single bond.

45. The oligomeric compound of any of claims 43-44, wherein the conjugate linker is cleavable.

46. The oligomeric compound of claim 43 or claim 45, wherein the conjugate linker comprises 1-3 linker-nucleosides.

47. The oligomeric compound of any of claims 43-45, wherein the conjugate linker does not comprise any linker nucleosides.

48. The oligomeric compound of any of claims 42-47, wherein the conjugate group is attached to the modified oligonucleotide at the 5′-end of the modified oligonucleotide.

49. The oligomeric compound of any of claims 42-47, wherein the conjugate group is attached to the modified oligonucleotide at the 3′-end of the modified oligonucleotide.

50. The oligomeric compound of any of claims 1 to 49, wherein the oligomeric compound comprises a terminal group.

51. The oligomeric compound of claim 50, wherein the terminal group is an abasic sugar moiety.

52. The oligomeric compound of any of claims 1-51, wherein the oligomeric compound is a singled-stranded oligomeric compound.

53. A chirally enriched population of oligomeric compounds of any of claims 1-52, wherein the population is enriched for modified oligonucleotides comprising at least one particular phosphorothioate internucleoside linkage having a particular stereochemical configuration.

54. The chirally enriched population of claim 53, wherein the population is enriched for modified oligonucleotides comprising at least one particular phosphorothioate internucleoside linkage having the (Sp) or (Rp) configuration.

55. The chirally enriched population of claim 53, wherein the population is enriched for modified oligonucleotides having a particular, independently selected stereochemical configuration at each phosphorothioate internucleoside linkage.

56. The chirally enriched population of claim 53, wherein the population is enriched for modified oligonucleotides having the (Rp) configuration at one particular phosphorothioate internucleoside linkage and the (Sp) configuration at each of the remaining phosphorothioate internucleoside linkages.

57. The chirally enriched population of claim 53, wherein the population is enriched for modified oligonucleotides having at least 3 contiguous phosphorothioate internucleoside linkages in the Sp, Sp, and Rp configurations, in the 5′ to 3′ direction.

58. A population of oligomeric compounds of any of claims 1-52, wherein all of the phosphorothioate internucleoside linkages of the modified oligonucleotide are stereorandom.

59. An oligomeric duplex, comprising a first oligomeric compound and a second oligomeric compound comprising a second modified oligonucleotide, wherein the first oligomeric compound is an oligomeric compound of any of claims 1-52.

60. The oligomeric duplex of claim 59, wherein the second modified oligonucleotide consists of 8 to 80 linked nucleosides, and wherein the nucleobase sequence of the second modified oligonucleotide comprises a complementary region of at least 8 nucleobases that is at least 90% complementary to the nucleobase sequence of an equal length portion of the first modified oligonucleotide.

61. The oligomeric duplex of claim 59 or claim 60, wherein the modified oligonucleotide of the first oligomeric compound comprises a 5′-stabilized phosphate group.

62. The oligomeric duplex of claim 61, wherein the stabilized phosphate group comprises a cyclopropyl phosphonate or a vinyl phosphonate.

63. The oligomeric duplex of any of claims 59-62, wherein the modified oligonucleotide of the first oligomeric compound comprises a glycol nucleic acid (GNA) sugar surrogate.

64. The oligomeric duplex of any of claims 59-63, wherein the modified oligonucleotide of the first oligomeric compound comprises a 2′-NMA sugar moiety.

65. The oligomeric duplex of any of claims 59-64, wherein at least one nucleoside of the second modified oligonucleotide comprises a modified sugar moiety.

66. The oligomeric duplex of claim 65, wherein the modified sugar moiety of the second modified oligonucleotide comprises a bicyclic sugar moiety.

67. The oligomeric duplex of claim 66, wherein the bicyclic sugar moiety of the second modified oligonucleotide comprises a 2′-4′ bridge selected from —O—CH₂—; and —O—CH(CH₃)—.

68. The oligomeric duplex of claim 66, wherein the modified sugar moiety of the second modified oligonucleotide comprises a non-bicyclic modified sugar moiety.

69. The oligomeric duplex of claim 68, wherein the non-bicyclic modified sugar moiety of the second modified oligonucleotide is a 2′-MOE sugar moiety, a 2′-F sugar moiety, or 2′-OMe sugar moiety.

70. The oligomeric duplex of any of claims 59-65, wherein at least one nucleoside of the second modified oligonucleotide comprises a sugar surrogate.

71. The oligomeric duplex of any of claims 59-70, wherein the second modified oligonucleotide comprises at least one modified internucleoside linkage.

72. The oligomeric duplex of claim 71, wherein at least one modified internucleoside linkage of the second modified oligonucleotide is a phosphorothioate internucleoside linkage.

73. The oligomeric duplex of any of claims 59-72, wherein at least one internucleoside linkage of the second modified oligonucleotide is a phosphodiester internucleoside linkage.

74. The oligomeric duplex of any of claims 59-73, wherein each internucleoside linkage of the second modified oligonucleotide is independently selected from a phosphodiester or a phosphorothioate internucleoside linkage.

75. The oligomeric duplex of any of claims 59-74, wherein the second modified oligonucleotide comprises at least one modified nucleobase.

76. The oligomeric duplex of claim 75, wherein the modified nucleobase of the second modified oligonucleotide is 5-methylcytosine.

77. The oligomeric duplex of any of claims 59-76, wherein the second modified oligonucleotide comprises a conjugate group.

78. The oligomeric duplex of claim 77, wherein the conjugate group comprises a conjugate linker and a conjugate moiety.

79. The oligomeric duplex of claim 77 or claim 78, wherein the conjugate group is attached to the second modified oligonucleotide at the 5′-end of the second modified oligonucleotide.

80. The oligomeric duplex of claim 77 or claim 78, wherein the conjugate group is attached to the second modified oligonucleotide at the 3′-end of the modified oligonucleotide.

81. The oligomeric duplex of claim 77 or claim 78, wherein the conjugate group is attached via the 2′ position of a ribosyl sugar moiety at an internal position of the second modified oligonucleotide.

82. The oligomeric duplex of any of claims 77-81, wherein the conjugate group comprises a lipid.

83. The oligomeric duplex of any of claims 77-82, wherein the conjugate group comprises a cell-targeting moiety.

84. The oligomeric duplex of any of claims 77-83, wherein the second modified oligonucleotide comprises a terminal group.

85. The oligomeric duplex of claim 84, wherein the terminal group is an abasic sugar moiety.

86. The oligomeric duplex of any of claims 59-85, wherein the second modified oligonucleotide consists of 10 to 25, 10 to 30, 10 to 50, 12 to 20, 12 to 25, 12 to 30, 12 to 50, 13 to 20, 13 to 25, 13 to 30, 13 to 50, 14 to 20, 14 to 25, 14 to 30, 14 to 50, 15 to 20, 15 to 25, 15 to 30, 15 to 50, 16 to 18, 16 to 20, 16 to 25, 16 to 30, 16 to 50, 17 to 20, 17 to 25, 17 to 30, 17 to 50, 18 to 20, 18 to 22, 18 to 25, 18 to 30, 18 to 50, 19 to 20, 19 to 25, 19 to 30, 19 to 50, 20 to 25, 20 to 30, 20 to 50, 21 to 25, 21 to 30, 21 to 50, 22 to 25, 22 to 30, 22 to 50, 23 to 25, 23 to 30, or 23 to 50 linked nucleosides.

87. An antisense agent comprising an antisense compound, wherein the antisense compound is the oligomeric compound of any of claims 1-52.

88. An antisense agent, wherein the antisense agent is the oligomeric duplex of any of claims 59-86.

89. The antisense agent of claim 87 or claim 88, wherein the antisense agent is:
i) an RNase H agent capable of reducing the amount of PTBP1 nucleic acid through the activation of RNase H; or
ii) an RNAi agent capable of reducing the amount of PTBP1 nucleic acid through the activation of RISC/Ago2.

90. The antisense agent of any of claims 87-89, wherein the antisense agent comprises a conjugate group, and wherein the conjugate group comprises a cell-targeting moiety.

91. A pharmaceutical composition comprising an oligomeric compound of any of claims 1-52, a population of any of claims 53-58, an oligomeric duplex of any of claims 59-86, or an antisense agent of any of claims 87-90, and a pharmaceutically acceptable diluent or carrier.

92. The pharmaceutical composition of claim 91, wherein the pharmaceutically acceptable diluent is phosphate-buffered saline (PBS) or artificial cerebrospinal fluid (aCSF).

93. The pharmaceutical composition of claim 92, wherein the pharmaceutical composition consists essentially of the oligomeric compound of any of claims 1-52, the population of any of claims 53-58, the oligomeric duplex of any of claims 59-86, or the antisense agent of any of claims 87-90, and phosphate-buffered saline (PBS).

94. The pharmaceutical composition of claim 92, wherein the pharmaceutical composition consists essentially of the oligomeric compound of any of claims 1-52, the population of any of claims 53-58, the oligomeric duplex of any of claims 59-86, or the antisense agent of any of claims 87-90 and artificial cerebrospinal fluid (aCSF).
